SEQUENCE LISTING (119 sequences)

SEQ ID NO: 1 - protein, 15 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXRK

SEQ ID NO: 2 - protein, 15 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-isoleucine
  SITE 3..13: note = Intrachain Disulfide bond
YICXXQDWXAHRXXK

SEQ ID NO: 3 - protein, 15 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 3..13: note = Intrachain Disulfide bond
YICXXXDWXAHRXRK

SEQ ID NO: 4 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-isoleucine
  SITE 3..13: note = Intrachain Disulfide bond
YICXXQDWXAHRXXKKK

SEQ ID NO: 5 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-isoleucine
  SITE 3..13: note = Intrachain Disulfide bond
YICXXQDWXAHRXXEEE

SEQ ID NO: 6 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-isoleucine
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXAHRXXEEE

SEQ ID NO: 7 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXTKKK

SEQ ID NO: 8 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXTEEE

SEQ ID NO: 9 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXAHRXTEEE

SEQ ID NO: 10 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXRKKK

SEQ ID NO: 11 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXREEE

SEQ ID NO: 12 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXAHRXREEE

SEQ ID NO: 13 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXLKKK

SEQ ID NO: 14 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXLEEE

SEQ ID NO: 15 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXAHRXLEEE

SEQ ID NO: 16 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXWEEE

SEQ ID NO: 17 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXAHRXWEEE

SEQ ID NO: 18 - protein, 18 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-isoleucine
  SITE 16: note = Gamma-L-glutamate
  SITE 17: note = Gamma-L-glutamate
  SITE 18: note = Gamma-L-glutamate
  SITE 3..13: note = Intrachain Disulfide bond
YICXXQDWXAHRXXKEEE

SEQ ID NO: 19 - protein, 18 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 16: note = Gamma-L-glutamate
  SITE 17: note = Gamma-L-glutamate
  SITE 18: note = Gamma-L-glutamate
  SITE 3..13: note = Intrachain Disulfide bond
YICXXQDWXAHRXWKEEE

SEQ ID NO: 20 - protein, 17 aa, synthetic construct
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated N-methyl-L-isoleucine
  SITE 6..16: note = Intrachain Disulfide bond
KKKYICXXQDWXAHRXX

SEQ ID NO: 21 - protein, 17 aa, synthetic construct
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated N-methyl-L-isoleucine
  SITE 6..16: note = Intrachain Disulfide bond
EEEYICXXQDWXAHRXX

SEQ ID NO: 22 - protein, 17 aa, synthetic construct
  SITE 1: note = Gamma-L-glutamate
  SITE 2: note = Gamma-L-glutamate
  SITE 3: note = Gamma-L-glutamate
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated N-methyl-L-isoleucine
  SITE 6..16: note = Intrachain Disulfide bond
EEEYICXXQDWXAHRXX

SEQ ID NO: 23 - protein, 17 aa, synthetic construct
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
KKKYICXXQDWXAHRXT

SEQ ID NO: 24 - protein, 17 aa, synthetic construct
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
EEEYICXXQDWXAHRXT

SEQ ID NO: 25 - protein, 17 aa, synthetic construct
  SITE 1: note = Gamma-L-glutamate
  SITE 2: note = Gamma-L-glutamate
  SITE 3: note = Gamma-L-glutamate
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
EEEYICXXQDWXAHRXT

SEQ ID NO: 26 - protein, 17 aa, synthetic construct
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
KKKYICXXQDWXAHRXR

SEQ ID NO: 27 - protein, 17 aa, synthetic construct
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
EEEYICXXQDWXAHRXR

SEQ ID NO: 28 - protein, 17 aa, synthetic construct
  SITE 1: note = Gamma-L-glutamate
  SITE 2: note = Gamma-L-glutamate
  SITE 3: note = Gamma-L-glutamate
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
EEEYICXXQDWXAHRXR

SEQ ID NO: 29 - protein, 17 aa, synthetic construct
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
KKKYICXXQDWXAHRXL

SEQ ID NO: 30 - protein, 17 aa, synthetic construct
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
EEEYICXXQDWXAHRXL

SEQ ID NO: 31 - protein, 17 aa, synthetic construct
  SITE 1: note = Gamma-L-glutamate
  SITE 2: note = Gamma-L-glutamate
  SITE 3: note = Gamma-L-glutamate
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
EEEYICXXQDWXAHRXL

SEQ ID NO: 32 - protein, 17 aa, synthetic construct
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
EEEYICXXQDWXAHRXW

SEQ ID NO: 33 - protein, 17 aa, synthetic construct
  SITE 1: note = Gamma-L-glutamate
  SITE 2: note = Gamma-L-glutamate
  SITE 3: note = Gamma-L-glutamate
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = Amidated D-amino acid
  SITE 6..16: note = Intrachain Disulfide bond
EEEYICXXQDWXAHRXW

SEQ ID NO: 34 - protein, 18 aa, synthetic construct
  SITE 1: note = Gamma-L-glutamate
  SITE 2: note = Gamma-L-glutamate
  SITE 3: note = Gamma-L-glutamate
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = N-methyl-L-isoleucine
  SITE 6..16: note = Intrachain Disulfide bond
EEEYICXXQDWXAHRXXK

SEQ ID NO: 35 - protein, 18 aa, synthetic construct
  SITE 1: note = Gamma-L-glutamate
  SITE 2: note = Gamma-L-glutamate
  SITE 3: note = Gamma-L-glutamate
  SITE 4: note = D-amino acid
  SITE 7: note = L-tert-butylglycine
  SITE 8: note = 1-methyl-L-tryptophan
  SITE 12: note = Sarcosine
  SITE 16: note = L-penicillamine
  SITE 17: note = D-amino acid
  SITE 6..16: note = Intrachain Disulfide bond
EEEYICXXQDWXAHRXWK

SEQ ID NO: 36 - protein, 17 aa, synthetic construct
  SITE 1: note = D-amino acid
  SITE 5: note = 1-methyl-L-tryptophan
  SITE 9: note = Sarcosine
  SITE 13: note = L-penicillamine
  SITE 14: note = N-methyl-L-isoleucine
  SITE 3..13: note = Intrachain Disulfide bond
YICVXQDWXAHRXXKKK

SEQ ID NO: 37 - protein, 17 aa, synthetic construct
  SITE 1: note = D-amino acid
  SITE 5: note = 1-methyl-L-tryptophan
  SITE 9: note = Sarcosine
  SITE 13: note = L-penicillamine
  SITE 14: note = N-methyl-L-isoleucine
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICVXQDWXAHRXXEEE

SEQ ID NO: 38 - protein, 17 aa, synthetic construct
  SITE 1: note = D-amino acid
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICVXQDWXAHRXTKKK

SEQ ID NO: 39 - protein, 17 aa, synthetic construct
  SITE 1: note = D-amino acid
  SITE 5: note = 1-methyl-L-tryptophan
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICVXQDWXAHRXTEEE

SEQ ID NO: 40 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-isoleucine
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXEHRXXEEE

SEQ ID NO: 41 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-isoleucine
  SITE 3..13: note = Intrachain Disulfide bond
YICXXQDWXEHRXXKKK

SEQ ID NO: 42 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXEHRXTEEE

SEQ ID NO: 43 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXEHRXTKKK

SEQ ID NO: 44 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXXDWXAHRXTEEE

SEQ ID NO: 45 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-phenylalanine
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXAHRXXEEE

SEQ ID NO: 46 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXAHRXPEEE

SEQ ID NO: 47 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXAHRXKEEE

SEQ ID NO: 48 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXKKKK

SEQ ID NO: 49 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXQDWXAHRXFEEE

SEQ ID NO: 50 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 3..13: note = Intrachain Disulfide bond
YICXXXDWXAHRXRKKK

SEQ ID NO: 51 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXXDWXAHRXREEE

SEQ ID NO: 52 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = N-methyl-L-isoleucine
  SITE 3..13: note = Intrachain Disulfide bond
YICXXXDWXAHRXXKKK

SEQ ID NO: 53 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 15: note = Gamma-L-glutamate
  SITE 16: note = Gamma-L-glutamate
  SITE 17: note = Gamma-L-glutamate
  SITE 3..13: note = Intrachain Disulfide bond
YICXXXDWXAHRXTEEE

SEQ ID NO: 54 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = N-methyl-L-isoleucine
  SITE 3..13: note = Intrachain Disulfide bond
YICXXXDWXEHRXXKKK

SEQ ID NO: 55 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXAHRXFKKK

SEQ ID NO: 56 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 3..13: note = Intrachain Disulfide bond
YICXXQDWXAHRXPKKK

SEQ ID NO: 57 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-phenylalanine
  SITE 3..13: note = Intrachain Disulfide bond
YICXXQDWXAHRXXKKK

SEQ ID NO: 58 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXEHRXKKKK

SEQ ID NO: 59 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 3..13: note = Intrachain Disulfide bond
YICXXQDWXEHRXPKKK

SEQ ID NO: 60 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = N-methyl-L-phenylalanine
  SITE 3..13: note = Intrachain Disulfide bond
YICXXQDWXEHRXXKKK

SEQ ID NO: 61 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXEHRXRKKK

SEQ ID NO: 62 - protein, 17 aa, synthetic construct
  SITE 1: note = D-amino acid
  SITE 4: note = L-tert-butylglycine
  SITE 5: note = 1-methyl-L-tryptophan
  SITE 9: note = Sarcosine
  SITE 13: note = L-penicillamine
  SITE 14: note = D-amino acid
  SITE 3..13: note = Intrachain Disulfide bond
YICXXQDWXEHRXFKKK

SEQ ID NO: 63 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 3..13: note = Intrachain Disulfide bond
YICXXXDWXAHRXTKKK

SEQ ID NO: 64 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 3..13: note = Intrachain Disulfide bond
YICXXXDWXAHRXKKKK

SEQ ID NO: 65 - skipped

SEQ ID NO: 66 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = N-methyl-L-phenylalanine
  SITE 3..13: note = Intrachain Disulfide bond
YICXXXDWXAHRXXKKK

SEQ ID NO: 67 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 3..13: note = Intrachain Disulfide bond
YICXXXDWXAHRXFKKK

SEQ ID NO: 68 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 3..13: note = Intrachain Disulfide bond
YICXXXDWXEHRXTKKK

SEQ ID NO: 69 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 3..13: note = Intrachain Disulfide bond
YICXXXDWXEHRXKKKK

SEQ ID NO: 70 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 3..13: note = Intrachain Disulfide bond
YICXXXDWXEHRXPKKK

SEQ ID NO: 71 - protein, 17 aa, synthetic construct
  SITE 1: note = D-amino acid
  SITE 4: note = L-tert-butylglycine
  SITE 5: note = 1-methyl-L-tryptophan
  SITE 6: note = (2S)-2-amino-3-acetamidopropanoic acid
  SITE 9: note = Sarcosine
  SITE 13: note = L-penicillamine
  SITE 14: note = D-amino acid
  SITE 3..13: note = Intrachain Disulfide bond
YICXXXDWXEHRXFKKK

SEQ ID NO: 72 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 14: note = D-amino acid
  SITE 3..13: note = Intrachain Disulfide bond
YICXXXDWXAHRXTKKK

SEQ ID NO: 73 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 14: note = D-amino acid
  SITE 3..13: note = Intrachain Disulfide bond
YICXXXDWXAHRXKKKK

SEQ ID NO: 74 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 3..13: note = Intrachain Disulfide bond
YICXXXDWXAHRXPKKK

SEQ ID NO: 75 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 14: note = D-amino acid
  SITE 3..13: note = Intrachain Disulfide bond
YICXXXDWXAHRXRKKK

SEQ ID NO: 76 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 14: note = D-amino acid
  SITE 3..13: note = Intrachain Disulfide bond
YICXXXDWXAHRXFKKK

SEQ ID NO: 77 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 14: note = D-amino acid
  SITE 3..13: note = Intrachain Disulfide bond
YICXXXDWXEHRXTKKK

SEQ ID NO: 78 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 14: note = D-amino acid
  SITE 3..13: note = Intrachain Disulfide bond
YICXXXDWXEHRXKKKK

SEQ ID NO: 79 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 14: note = 4-amino-piperidine-4 carboxylic acid
  SITE 3..13: note = Intrachain Disulfide bond
YICXXXDWXEHRXXKKK

SEQ ID NO: 80 - protein, 17 aa, synthetic construct
  SITE 1: note = D-amino acid
  SITE 4: note = L-tert-butylglycine
  SITE 5: note = 1-methyl-L-tryptophan
  SITE 6: note = Beta-homo-L-asparagine
  SITE 9: note = Sarcosine
  SITE 13: note = L-penicillamine
  SITE 14: note = D-amino acid
  SITE 3..13: note = Intrachain Disulfide bond
YICXXXDWXEHRXFKKK

SEQ ID NO: 81 - protein, 17 aa, synthetic construct
  SITE 4: note = D-amino acid
  SITE 7: note = L-tert-butylglycine
  SITE 8: note = 1-methyl-L-tryptophan
  SITE 9: note = (2S)-2-amino-3-acetamidopropanoic acid
  SITE 12: note = Sarcosine
  SITE 16: note = L-penicillamine
  SITE 17: note = D-amino acid
  SITE 6..16: note = Intrachain Disulfide bond
KKKYICXXXDWXAHRXR

SEQ ID NO: 82 - protein, 17 aa, synthetic construct
  SITE 4: note = D-amino acid
  SITE 7: note = L-tert-butylglycine
  SITE 8: note = 1-methyl-L-tryptophan
  SITE 9: note = (2S)-2-amino-3-acetamidopropanoic acid
  SITE 12: note = Sarcosine
  SITE 16: note = L-penicillamine
  SITE 17: note = D-amino acid
  SITE 6..16: note = Intrachain Disulfide bond
KKKYICXXXDWXAHRXT

SEQ ID NO: 83 - protein, 17 aa, synthetic construct
  SITE 4: note = D-amino acid
  SITE 7: note = L-tert-butylglycine
  SITE 8: note = 1-methyl-L-tryptophan
  SITE 9: note = (2S)-2-amino-3-acetamidopropanoic acid
  SITE 12: note = Sarcosine
  SITE 16: note = L-penicillamine
  SITE 17: note = D-amino acid
  SITE 6..16: note = Intrachain Disulfide bond
KKKYICXXXDWXAHRXK

SEQ ID NO: 84 - protein, 17 aa, synthetic construct
  SITE 4: note = D-amino acid
  SITE 7: note = L-tert-butylglycine
  SITE 8: note = 1-methyl-L-tryptophan
  SITE 9: note = (2S)-2-amino-3-acetamidopropanoic acid
  SITE 12: note = Sarcosine
  SITE 16: note = L-penicillamine
  SITE 6..16: note = Intrachain Disulfide bond
KKKYICXXXDWXAHRXP

SEQ ID NO: 85 - protein, 17 aa, synthetic construct
  SITE 4: note = D-amino acid
  SITE 7: note = L-tert-butylglycine
  SITE 8: note = 1-methyl-L-tryptophan
  SITE 9: note = (2S)-2-amino-3-acetamidopropanoic acid
  SITE 12: note = Sarcosine
  SITE 16: note = L-penicillamine
  SITE 17: note = D-amino acid
  SITE 6..16: note = Intrachain Disulfide bond
KKKYICXXXDWXAHRXF

SEQ ID NO: 86 - protein, 17 aa, synthetic construct
  SITE 4: note = D-amino acid
  SITE 7: note = L-tert-butylglycine
  SITE 8: note = 1-methyl-L-tryptophan
  SITE 9: note = (2S)-2-amino-3-acetamidopropanoic acid
  SITE 12: note = Sarcosine
  SITE 16: note = L-penicillamine
  SITE 17: note = D-amino acid
  SITE 6..16: note = Intrachain Disulfide bond
KKKYICXXXDWXEHRXR

SEQ ID NO: 87 - protein, 17 aa, synthetic construct
  SITE 4: note = D-amino acid
  SITE 7: note = L-tert-butylglycine
  SITE 8: note = 1-methyl-L-tryptophan
  SITE 9: note = (2S)-2-amino-3-acetamidopropanoic acid
  SITE 12: note = Sarcosine
  SITE 16: note = L-penicillamine
  SITE 17: note = D-amino acid
  SITE 6..16: note = Intrachain Disulfide bond
KKKYICXXXDWXEHRXT

SEQ ID NO: 88 - protein, 17 aa, synthetic construct
  SITE 4: note = D-amino acid
  SITE 7: note = L-tert-butylglycine
  SITE 8: note = 1-methyl-L-tryptophan
  SITE 9: note = (2S)-2-amino-3-acetamidopropanoic acid
  SITE 12: note = Sarcosine
  SITE 16: note = L-penicillamine
  SITE 17: note = D-amino acid
  SITE 6..16: note = Intrachain Disulfide bond
KKKYICXXXDWXEHRXK

SEQ ID NO: 89 - protein, 17 aa, synthetic construct
  SITE 4: note = D-amino acid
  SITE 7: note = L-tert-butylglycine
  SITE 8: note = 1-methyl-L-tryptophan
  SITE 9: note = Beta-homo-L-asparagine
  SITE 12: note = Sarcosine
  SITE 16: note = L-penicillamine
  SITE 17: note = D-amino acid
  SITE 6..16: note = Intrachain Disulfide bond
KKKYICXXXDWXEHRXR

SEQ ID NO: 90 - protein, 17 aa, synthetic construct
  SITE 4: note = D-amino acid
  SITE 7: note = L-tert-butylglycine
  SITE 8: note = 1-methyl-L-tryptophan
  SITE 9: note = Beta-homo-L-asparagine
  SITE 12: note = Sarcosine
  SITE 16: note = L-penicillamine
  SITE 17: note = D-amino acid
  SITE 6..16: note = Intrachain Disulfide bond
KKKYICXXXDWXEHRXT

SEQ ID NO: 91 - protein, 17 aa, synthetic construct
  SITE 4: note = D-amino acid
  SITE 7: note = L-tert-butylglycine
  SITE 8: note = 1-methyl-L-tryptophan
  SITE 9: note = Beta-homo-L-asparagine
  SITE 12: note = Sarcosine
  SITE 16: note = L-penicillamine
  SITE 17: note = D-amino acid
  SITE 6..16: note = Intrachain Disulfide bond
KKKYICXXXDWXEHRXK

SEQ ID NO: 92 - protein, 17 aa, synthetic construct
  SITE 4: note = D-amino acid
  SITE 7: note = L-tert-butylglycine
  SITE 8: note = 1-methyl-L-tryptophan
  SITE 9: note = Beta-homo-L-asparagine
  SITE 12: note = Sarcosine
  SITE 16: note = L-penicillamine
  SITE 6..16: note = Intrachain Disulfide bond
KKKYICXXXDWXEHRXP

SEQ ID NO: 93 - protein, 17 aa, synthetic construct
  SITE 4: note = D-amino acid
  SITE 7: note = L-tert-butylglycine
  SITE 8: note = 1-methyl-L-tryptophan
  SITE 9: note = Beta-homo-L-asparagine
  SITE 12: note = Sarcosine
  SITE 16: note = L-penicillamine
  SITE 17: note = D-amino acid
  SITE 6..16: note = Intrachain Disulfide bond
KKKYICXXXDWXEHRXF

SEQ ID NO: 94 - protein, 17 aa, synthetic construct
  SITE 1: note = D-amino acid
  SITE 4: note = L-tert-butylglycine
  SITE 5: note = 1-methyl-L-tryptophan
  SITE 9: note = sarcosine
  SITE 13: note = L-penicillamine
  SITE 14..17: note = D-amino acid
  CROSSLNK 3..13: note = intrachain disulfide bond
YICXXQDWXAHRXTKKK

SEQ ID NO: 95 - protein, 17 aa, synthetic construct
  CROSSLNK 3..13: note = intrachain disulfide bond
  SITE 1: note = D-amino acid
  SITE 4: note = L-tert-butylglycine
  SITE 6: note = beta-homo-L-asparagine
  SITE 5: note = 1-methyl-L-tryptophan
  SITE 9: note = sarcosine
  SITE 13: note = L-penicillamine
  SITE 14: note = D-amino acid
  SITE 15..17: note = gamma-D-glutamate
YICXXXDWXAHRXTXXX

SEQ ID NO: 96 - protein, 17 aa, synthetic construct
  CROSSLNK 3..13: note = intrachain disulfide bond
  SITE 14..17: note = D-amino acid
  SITE 1: note = D-amino acid
  SITE 4: note = L-tert-butylglycine
  SITE 5: note = 1-methyl-L-tryptophan
  SITE 6: note = beta-homo-L-asparagine
  SITE 9: note = sarcosine
  SITE 13: note = L-penicillamine
YICXXXDWXAHRXKKKK

SEQ ID NO: 97 - protein, 17 aa, synthetic construct
  SITE 1: note = D amino acid
  SITE 14..17: note = D-amino acid
  CROSSLNK 3..13: note = intrachain disulfide bond
  SITE 4: note = L-tert-butylglycine
  SITE 5: note = 1-methyl-L-tryptophan
  SITE 6: note = (2S)-2-amino-3-acetamidopropanoic acid
  SITE 9: note = sarcosine
  SITE 13: note = L-penicillamine
YICXXXDWXEHRXTKKK

SEQ ID NO: 98 - protein, 16 aa, synthetic construct
  CROSSLNK 3..13: note = intrachain disulfide bond
  SITE 14: note = D amino acid
  SITE 1: note = D amino acid
  SITE 4: note = L-tert-butylglycine
  SITE 5: note = 1-methyl-L-tryptophan
  SITE 6: note = beta-homo-L-asparagine
  SITE 9: note = sarcosine
  SITE 13: note = L-penicillamine
  SITE 15..16: note = gamma-L-glutamate
YICXXXDWXAHRXTXX

SEQ ID NO: 99 - protein, 15 aa, synthetic construct
  SITE 1: note = D amino acid
  CROSSLNK 3..13: note = intrachain disulfide bond
  SITE 4: note = L-tert-butylglycine
  SITE 5: note = 1-methyl-L-tryptophan
  SITE 6: note = beta-homo-L-asparagine
  SITE 9: note = sarcosine
  SITE 13: note = L-penicillamine
  SITE 14: note = D amino acid
  SITE 15: note = gamma-L-glutamate
YICXXXDWXAHRXTX

SEQ ID NO: 100 - protein, 16 aa, synthetic construct
  SITE 1: note = D amino acid
  CROSSLNK 3..13: note = intrachain disulfide bond
  SITE 4: note = L-tert-butylglycine
  SITE 5: note = 1-methyl-L-tryptophan
  SITE 6: note = beta-homo-L-asparagine
  SITE 9: note = sarcosine
  SITE 13: note = L-penicillamine
  SITE 14: note = D amino acid
YICXXXDWXAHRXKKK

SEQ ID NO: 101 - protein, 14 aa, synthetic construct
  SITE 1: note = D-amino acid
  SITE 5: note = 1-methyl-L-tryptophan
  SITE 9: note = Sarcosine
  SITE 14: note = Amidated N-methyl-L-isoleucine
  SITE 3..13: note = Intrachain Disulfide bond
YICVXQDWXAHRCX

SEQ ID NO: 102 - protein, 17 aa, synthetic construct
  SITE 1: note = D-amino acid
  SITE 5: note = 1-methyl-L-tryptophan
  SITE 9: note = Sarcosine
  SITE 14: note = N-methyl-L-isoleucine
  SITE 17: note = Amidated residue
  SITE 3..13: note = Intrachain Disulfide bond
YICVXQDWXAHRCXKKK

SEQ ID NO: 103 - protein, 27 aa, synthetic construct
  SITE 2..12: note = Intrachain Disulfide bond
ICVVQDWGHHRCTAGHMANLTSHASAI

SEQ ID NO: 104 - protein, 13 aa, synthetic construct
  SITE 2..12: note = Intrachain Disulfide bond
  SITE 13: note = Amidated residue
ICVVQDWGHHRCT

SEQ ID NO: 105 - protein, 18 aa, synthetic construct
  SITE 1: note = D-amino acid
  VARIANT 4: note = L-tert-butylglycine or V
  SITE 5: note = 1-methyl-L-tryptophan
  VARIANT 6: note = Q, (2S)-2-amino-3-acetamidopropanoic acid, or Beta-homo-L-asparagine
  SITE 9: note = Sarcosine
  VARIANT 10: note = A or E
  SITE 13: note = L-penicillamine
  VARIANT 14: note = N-methyl-L-isoleucine, D-Arg, D-Lys, D-Thr, D-Phe, D-Trp, P, D-Leu, N-methyl-L-phenylalanine, or 4-amino-piperidine-4 carboxylic acid
  VARIANT 15: note = K, E, or Gamma-L-glutamate
  VARIANT 16: note = K, E, or Gamma-L-glutamate
  VARIANT 17: note = K, E, or Gamma-L-glutamate
  VARIANT 18: note = K, E, or Gamma-L-glutamate
  SITE 3..13: note = Intrachain Disulfide bond
  VARIANT 15..18: note = This region may encompass 1-4 residues
YICXXXDWXXHRXXXXXX

SEQ ID NO: 106 - protein, 18 aa, synthetic construct
  SITE 1: note = D-amino acid
  VARIANT 4: note = L-tert-butylglycine or V
  SITE 5: note = 1-methyl-L-tryptophan
  VARIANT 6: note = Q, (2S)-2-amino-3-acetamidopropanoic acid, or Beta-homo-L-asparagine
  SITE 9: note = Sarcosine
  VARIANT 10: note = A or E
  SITE 13: note = L-penicillamine
  VARIANT 14: note = N-methyl-L-isoleucine, D-Arg, D-Lys, D-Thr, D-Phe, D-Trp, P, D-Leu, N-methyl-L-phenylalanine, or 4-amino-piperidine-4 carboxylic acid
  VARIANT 15: note = K, E, or Gamma-L-glutamate
  VARIANT 16: note = K, E, or Gamma-L-glutamate
  VARIANT 17: note = K, E, or Gamma-L-glutamate
  VARIANT 18: note = K, E, or Gamma-L-glutamate
  SITE 3..13: note = Intrachain Disulfide bond
  VARIANT 15..18: note = This region may encompass 1-4 residues
YICXXXDWXXHRXXXXXX

SEQ ID NO: 107 - protein, 19 aa, synthetic construct
  VARIANT 1: note = K, E, or Gamma-L-glutamate
  VARIANT 2: note = K, E, or Gamma-L-glutamate
  VARIANT 3: note = K, E, or Gamma-L-glutamate
  VARIANT 4: note = K, E, or Gamma-L-glutamate
  SITE 5: note = D-amino acid
  VARIANT 8: note = L-tert-butylglycine or V
  SITE 9: note = 1-methyl-L-tryptophan
  VARIANT 10: note = Q, (2S)-2-amino-3-acetamidopropanoic acid, or Beta-homo-L-asparagine
  SITE 13: note = Sarcosine
  VARIANT 14: note = A or E
  SITE 17: note = L-penicillamine
  VARIANT 18: note = N-methyl-L-isoleucine, D-Arg, D-Lys, D-Thr, D-Phe, D-Trp, P, D-Leu, N-methyl-L-phenylalanine, or 4-amino-piperidine-4 carboxylic acid, which may be amidated if position 19 is absent
  VARIANT 19: note = May be absent
  VARIANT 1..4: note = This region may encompass 1-4 residues
  SITE 7..17: note = Intrachain Disulfide bond
XXXXYICXXXDWXXHRXXK

SEQ ID NO: 108 - protein, 18 aa, synthetic construct
  SITE 1: note = D-amino acid
  VARIANT 4: note = L-tert-butylglycine or V
  SITE 5: note = 1-methyl-L-tryptophan
  VARIANT 6: note = Q, (2S)-2-amino-3-acetamidopropanoic acid, or Beta-homo-L-asparagine
  SITE 9: note = Sarcosine
  VARIANT 10: note = A or E
  SITE 13: note = L-penicillamine
  SITE 14: note = D-amino acid
  VARIANT 15: note = E or Gamma-L-glutamate
  VARIANT 16: note = E or Gamma-L-glutamate
  VARIANT 17: note = E or Gamma-L-glutamate
  VARIANT 18: note = E or Gamma-L-glutamate
  SITE 3..13: note = Intrachain Disulfide bond
  VARIANT 15..18: note = This region may encompass 1-4 residues
YICXXXDWXXHRXWXXXX

SEQ ID NO: 109 - protein, 19 aa, synthetic construct
  VARIANT 1: note = K, E, or Gamma-L-glutamate
  VARIANT 2: note = K, E, or Gamma-L-glutamate
  VARIANT 3: note = K, E, or Gamma-L-glutamate
  VARIANT 4: note = K, E, or Gamma-L-glutamate
  SITE 5: note = D-amino acid
  VARIANT 8: note = L-tert-butylglycine or V
  SITE 9: note = 1-methyl-L-tryptophan
  VARIANT 10: note = Q, (2S)-2-amino-3-acetamidopropanoic acid, or Beta-homo-L-asparagine
  SITE 13: note = Sarcosine
  VARIANT 14: note = A or E
  SITE 17: note = L-penicillamine
  VARIANT 18: note = N-methyl-L-isoleucine, D-Arg, D-Lys, D-Thr, D-Phe, D-Trp, P, D-Leu, N-methyl-L-phenylalanine, or 4-amino-piperidine-4 carboxylic acid, which may be amidated if position 19 is absent
  VARIANT 19: note = May be absent
  VARIANT 1..4: note = This region may encompass 1-4 residues
  SITE 7..17: note = Intrachain Disulfide bond
XXXXYICXXXDWXXHRXXK

SEQ ID NO: 110 - protein, 19 aa, synthetic construct
  VARIANT 1: note = E or Gamma-L-glutamate
  VARIANT 2: note = E or Gamma-L-glutamate
  VARIANT 3: note = E or Gamma-L-glutamate
  VARIANT 4: note = E or Gamma-L-glutamate
  SITE 5: note = D-amino acid
  VARIANT 8: note = L-tert-butylglycine or V
  SITE 9: note = 1-methyl-L-tryptophan
  VARIANT 10: note = Q, (2S)-2-amino-3-acetamidopropanoic acid, or Beta-homo-L-asparagine
  SITE 13: note = Sarcosine
  VARIANT 14: note = A or E
  SITE 17: note = L-penicillamine
  VARIANT 18: note = D-amino acid which may be amidated if position 19 is absent
  VARIANT 19: note = May be absent
  VARIANT 1..4: note = This region may encompass 1-4 residues
  SITE 7..17: note = Intrachain Disulfide bond
XXXXYICXXXDWXXHRXWK

SEQ ID NO: 111 - protein, 18 aa, synthetic construct
  SITE 1: note = D-amino acid
  VARIANT 4: note = L-tert-butylglycine or V
  SITE 5: note = 1-methyl-L-tryptophan
  VARIANT 6: note = Q, (2S)-2-amino-3-acetamidopropanoic acid, or Beta-homo-L-asparagine
  SITE 9: note = Sarcosine
  VARIANT 10: note = A or E
  SITE 13: note = L-penicillamine
  SITE 14: note = D-amino acid
  VARIANT 15: note = E or Gamma-L-glutamate
  VARIANT 16: note = E or Gamma-L-glutamate
  VARIANT 17: note = E or Gamma-L-glutamate
  VARIANT 18: note = E or Gamma-L-glutamate
  SITE 3..13: note = Intrachain Disulfide bond
  VARIANT 15..18: note = This region may encompass 1-4 residues
YICXXXDWXXHRXWXXXX

SEQ ID NO: 112 - protein, 19 aa, synthetic construct
  VARIANT 1: note = E or Gamma-L-glutamate
  VARIANT 2: note = E or Gamma-L-glutamate
  VARIANT 3: note = E or Gamma-L-glutamate
  VARIANT 4: note = E or Gamma-L-glutamate
  SITE 5: note = D-amino acid
  VARIANT 8: note = L-tert-butylglycine or V
  SITE 9: note = 1-methyl-L-tryptophan
  VARIANT 10: note = Q, (2S)-2-amino-3-acetamidopropanoic acid, or Beta-homo-L-asparagine
  SITE 13: note = Sarcosine
  VARIANT 14: note = A or E
  SITE 17: note = L-penicillamine
  VARIANT 18: note = D-amino acid which may be amidated if position 19 is absent
  VARIANT 19: note = May be absent
  VARIANT 1..4: note = This region may encompass 1-4 residues
  SITE 7..17: note = Intrachain Disulfide bond
XXXXYICXXXDWXXHRXWK

SEQ ID NO: 113 - protein, 15 aa, synthetic construct
  SITE 1: note = D amino acid
  CROSSLNK 3..13: note = intrachain disulfide bond
  SITE 4: note = L-tert-butylglycine
  SITE 5: note = 1-methyl-L-tryptophan
  SITE 6: note = beta-homo-L-asparagine
  SITE 9: note = sarcosine
  SITE 13: note = L-penicillamine
  SITE 14: note = D amino acid
YICXXXDWXAHRXKK

SEQ ID NO: 114 - protein, 14 aa, synthetic construct
  SITE 1: note = D amino acid
  SITE 14: note = D amino acid
  CROSSLNK 3..13: note = intrachain disulfide bond
  SITE 4: note = L-tert-butylglycine
  SITE 5: note = 1-methyl-L-tryptophan
  SITE 6: note = beta-homo-L-asparagine
  SITE 9: note = sarcosine
  SITE 13: note = L-penicillamine
YICXXXDWXAHRXT

SEQ ID NO: 115 - protein, 14 aa, synthetic construct
  SITE 1: note = D amino acid
  SITE 14: note = D amino acid
  CROSSLNK 3..13: note = intrachain disulfide bond
  SITE 4: note = L-tert-butylglycine
  SITE 5: note = 1-methyl-L-tryptophan
  SITE 6: note = beta-homo-L-asparagine
  SITE 9: note = sarcosine
  SITE 13: note = L-penicillamine
YICXXXDWXAHRXK

SEQ ID NO: 116 - protein, 13 aa, synthetic construct
  SITE 1: note = residue is acetylated at the N terminus
  CROSSLNK 2..12: note = intrachain disulfide bond
  SITE 4: note = 1-methyl-L-tryptophan
  SITE 12: note = L-penicillamine
  SITE 13: note = amidated residue
ICVXQDWGAHRXT

SEQ ID NO: 117 - protein, 18 aa, synthetic construct
  SITE 1: note = D amino acid
  SITE 4: note = L-tert-butylglycine
  SITE 5: note = 1-methyl-L-tryptophan
  VARIANT 6: note = beta-homo-L-asparagine or (2S)-2-amino-3-acetamidopropanoic acid
  SITE 9: note = sarcosine
  VARIANT 10: note = A or E
  SITE 13: note = L-penicillamine
  VARIANT 14: note = D-Arg, D-Lys, D-Thr, D-Phe, P, or N-methyl-L-phenylalanine
  VARIANT 15: note = K, E, gamma-L-glutamate, D-Lys, or gamma-D-glutamate
  VARIANT 16: note = K, E, gamma-L-glutamate, D-Lys, or gamma-D-glutamate
  VARIANT 17: note = K, E, gamma-L-glutamate, D-Lys, or gamma-D-glutamate
  VARIANT 18: note = K, E, gamma-L-glutamate, D-Lys, or gamma-D-glutamate
  VARIANT 15..18: note = This region may encompass 1 to 4 residues
  CROSSLNK 3..13: note = intrachain disulfide bond
YICXXXDWXXHRXXXXXX

SEQ ID NO: 118 - protein, 19 aa, synthetic construct
  VARIANT 18: note = D-Arg, D-Lys, D-Thr, D-Phe, P, or N-methyl-L-phenylalanine
  VARIANT 1: note = K, E, or gamma-L-glutamate
  VARIANT 1..4: note = This region may encompass 1 to 4 residues
  VARIANT 2: note = K, E, or gamma-L-glutamate
  VARIANT 3: note = K, E, or gamma-L-glutamate
  VARIANT 4: note = K, E, or gamma-L-glutamate
  SITE 5: note = D amino acid
  SITE 8: note = L-tert-butyl-glycine
  SITE 9: note = 1-methyl-L-tryptophan
  VARIANT 10: note = (2S)-2-amino-3-acetamidopropanoic acid, or Beta-homo-L-asparagine
  SITE 13: note = sarcosine
  VARIANT 14: note = A or E
  SITE 17: note = L-penicillamine
  VARIANT 19: note = may be absent
  CROSSLNK 7..17: note = intrachain disulfide bond
XXXXYICXXXDWXXHRXXK

SEQ ID NO: 119 - protein, 17 aa, synthetic construct
  CROSSLNK 3..13: note = intrachain disulfide bond
  SITE 1: note = D amino acid
  SITE 4: note = L-tert-butylglycine
  SITE 5: note = 1-methyl-L-tryptophan
  VARIANT 6: note = Q, (2S)-2-amino-3-acetamidopropanoic acid, or Beta-homo-L-asparagine
  SITE 9: note = sarcosine
  VARIANT 10: note = A or E
  SITE 13: note = L-penicillamine
  VARIANT 14: note = D-Lysine or D-Threonine
  VARIANT 15: note = D-Lysine or gamma-D-Glutamate
  VARIANT 16: note = D-Lysine or gamma-D-Glutamate
  VARIANT 17: note = D-Lysine or gamma-D-Glutamate
YICXXXDWXXHRXXXXX

(19) United States
(12) Patent Application Publication
(10) Pub. No.: US 2026/0234189 A1
(43) Pub. Date: Aug. 13, 2026

(54) NOVEL AND STABLE COMPSTATIN ANALOGUES

(71) Applicant: Boehringer Ingelheim International GmbH, Ingelheim am Rhein (DE)

(72) Inventors: Daniel Paul TEUFEL, Hergatz (DE); John Ellsworth BROWN III, Neu-Ulm (DE); Stefan PETERS, Biberach an der Riss (DE); Holger WAGNER, Mettenberg (DE); Ali Munaim Yousif YOUSIF, Ochsenhausen (DE)

(21) Appl. No.: 19/457,461

(22) Filed: Jan. 23, 2026

(30) Foreign Application Priority Data
Jan. 24, 2025 (EP) 25153772.6

(51) Int. Cl. C07K 7/08; A61K 38/00

(52) U.S. Cl. CPC C07K 7/08; A61K 38/00

(57) ABSTRACT

The present invention relates to soluble and chemical stable compstatin analogues that are capable of binding to C3 protein and inhibiting complement activation. Pharmaceutical compositions comprising the compstatin analogues of the invention and the medical use thereof are also disclosed.

### RELATED APPLICATION DISCLOSURE

This application claims the benefit under 35 U.S.C. § 119 of EP Application No. EP25153772.6 filed 24 Jan. 2025, which is hereby incorporated by reference herein in its entirety.

### SEQUENCE DISCLOSURE

This application includes, as part of its disclosure, a “Sequence Listing XML” pursuant to 37 C.F.R. § 1.831(a) which is submitted in XML file format via the USPTO patent electronic filing system in a file named “01-3623-US-1-2025-12-1.xml”, created on Dec. 15, 2025, and having a size of 469000 bytes, which is hereby incorporated by reference herein in its entirety.

### FIELD OF THE INVENTION

The present invention relates to compstatin analogues that are capable of binding to C3 protein and inhibiting complement activation and to their medical use in the treatment of diseases or disorders characterized by unwanted activation of the complement cascade, such as eye or ocular diseases. Pharmaceutical compositions comprising a compstatin analogue of the invention are also disclosed.

### BACKGROUND INFORMATION

The human complement system is an important player in the defense against pathogenic organisms and the mediation of immune responses. Complement can be activated through three different pathways: the classical, lectin and alternative pathways. The major activation event that is shared by all three pathways is the proteolytic cleavage of the central protein of the complement system, C3, into its activation products C3a and C3b by C3 convertases. Generation of these fragments leads to the opsonization of pathogenic cells by C3b and iC3b, a process that renders them susceptible to phagocytosis or clearance, and to the activation of immune cells through an interaction with complement. Deposition of C3b on target cells also induces the formation of new convertase complexes and thereby initiates a self-amplification loop. An ensemble of plasma and cell surface-bound proteins carefully regulates complement activation to prevent host cells from self-attack by the complement cascade. However, excessive activation or inappropriate regulation of complement can lead to pathologic conditions, ranging from autoimmune to inflammatory diseases. The development of therapeutic complement inhibitors is therefore highly desirable. In this context, C3 and C3b have emerged as promising targets because their central role in the cascade allows for the simultaneous inhibition of the initiation, amplification, and downstream activation of complement.

In view of the therapeutic potential, there is a need for novel potent compstatin analogues that are capable of binding to C3 protein and inhibiting complement activation that are also soluble and stable at or around physiological pH, and therefore viable for human therapy via parenteral administration, particularly for administration via intravitreal injection.

WO1997/33603, WO2007/062249, WO2010/127336, WO2013/036778, WO2014/100407, WO2014/152391, WO2015/142701, WO2014/078731, WO2019/166411, WO2021/037942, WO2019/195712, WO9733603, WO9913899, WO2004/026328, WO2012/040259, WO2022/013374, as well as CN118834265A disclose compstatin analogues and the medical use thereof.

### SUMMARY OF THE INVENTION

The present invention related to peptide compounds (i.e. compstatin analogues) represented by formula (I) that are capable of binding to C3 protein and inhibiting complement activation (i.e. inhibit complement factor C3).

Particularly, the present invention provides peptide compounds that inhibit complement factor C3, are highly soluble at or around physiological pH, and highly chemically stable at or around physiological pH, in order to provide effective treatments for the diseases mentioned in the present specification, preferably for the treatment by parenteral administration, most preferably for the treatment of eye or ocular diseases by intravitreal (IVT) administration, for example via IVT injection or the like.

In the present context, the term “at or around physiological pH” means at or around pH 7.4, e.g. pH 7.4±0.5. Peptide therapeutics with high solubility at or around physiological pH may be formulated in liquid formulation in high dose in a small amount of volume.

Peptide therapeutics with high chemical stability at or around physiological pH may reach an extended duration of action when administered in an environment having circa a physiological pH.

Peptide therapeutics with high chemical stability at or around physiological pH may be stored for long period of time in liquid formulation having physiological pH.

Particularly, an aim of the present invention is to provide peptide compounds that inhibit complement factor C3, are soluble and chemically stable at or around physiological pH and are suitable for intravitreal administration, preferably IVT injection, to humans once monthly, or even better, once every two, three, or several months. In order to improve patient compliance to treatment of eye or ocular diseases by intravitreal administration, for example IVT injection, it is desirable to develop peptides suitable to be administered once monthly or even better once every several months, preferably every six or twelve months. Such extended intervals between two consequent intravitreal administrations require the development of peptides that not only bind with high affinity to C3 protein but also are soluble and highly chemically stable at or around physiological pH. In fact, peptide therapeutics with high chemical stability at or around physiological pH may reach an extended duration of action in the eye. Moreover, peptide therapeutics for intravitreal administration, for example IVT injection, are usually provided as pharmaceutical liquid formulations that have limited application volumes (e.g. 50-100 μL); therefore, good solubility of the peptides at or around physiological pH is a requirement for obtaining such formulation, particularly if a high drug load in the formulation or device used for administering the formulation (e.g. prefilled syringe) is needed.

A further aim of the present invention is to provide peptides that inhibit complement factor C3, are soluble and chemically stable at or around physiological pH, and suitable for a safe and tolerable treatment of humans, preferably for the treatment of eye or ocular diseases by intravitreal administration, such as IVT injection.

A further aim of the present invention is to provide peptide compounds that inhibit complement factor C3, soluble and chemically stable at or around physiological pH, and photo stable in a liquid formulation suitable for intravitreal administration, for example IVT injection, to humans.

In a first aspect, the invention provides compounds of formula (I) or salt thereof, preferably a pharmaceutically acceptable salt thereof,

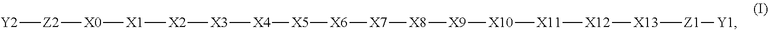

wherein X0 to X13, Y1, Y2, Z1, and Z2 are as defined in the present specification, and wherein the compound of formula (I) has a disulphide bridge between the side chains of X2 and X12.

In a second aspect, the invention relates to a pharmaceutical composition, preferably suitable for intravitreal injection, comprising at least one compound according to the first aspect together with one or more pharmaceutically acceptable carrier and/or excipients.

In a third aspect, the invention relates to compounds according to the first aspect for use as a medicament, preferably for use in a method of treatment of a disease or disorder disclosed in the present specification.

In a fourth aspect, the invention relates to a method for the treatment of a disease or disorder disclosed in the present specification, said method comprising administering a therapeutically effective amount of a compound of the present invention according to any of the aspects and embodiments disclosed herein, to a human in need thereof.

In a fifth aspect, the invention relates to a method for preparing compounds of the present invention according to any of the aspects and embodiments disclosed herein. The method may comprise the steps of synthesizing the compound of the invention by solid-phase or liquid-phase methodology, and optionally isolating and/or purifying the final product.

### Terms, Definitions and Conventions

Definitions of the residues X0 to X13, Y1, Y2, Z1, and Z2 will be given hereinafter as embodiments of the invention. Any and each of these definitions and embodiments may be combined with one another.

Terms not specifically defined herein should be given the meanings that would be given to them by one skilled in the art in light of the disclosure and the context. As used in the specification, however, unless specified to the contrary, the following terms have the meanings indicated and the following conventions are adhered to.

### Peptide and Amino Acids

The term “peptide”, as used herein, refers to a compound which comprises a series of amino acids interconnected by amide (or peptide) bonds.

The term “amino acid” refers to molecules containing an amino and a carboxylic acid group, and, optionally, one or more additional groups, often referred to as side chain. The term “amino acid” refers to any amino acid naturally occurring (including the 20 standard amino acids which are encoded by the standard genetic code in humans) or not naturally occurring. According to the present invention, unless otherwise stated, the amino acids are all L-amino acids (L-stereoisomer, natural amino acids). However, in some instances, D-configuration amino acids may be incorporated. In the present specification, unless naturally occurring amino acids are referred to by their full name (e.g. alanine, arginine, etc.), they are designated by their conventional three-letter or single-letter abbreviations (e.g. Arg or R for arginine, etc.). In the present context, a term having a “D” before a naturally occurring amino acid conventional three-letter code represents the D-configuration of such naturally occurring amino acid (e.g. “DArg” represents the D-configuration of arginine (Arg), and DLeu, DLys, DPhe, DThr, DTrp, DgGlu represent the D-configuration of Leu, Lys, Phe, Thr, Trp, and gGlu respectively, particularly, DgGlu refers to the residue of gamma-D-glutamate). In the cases of less common or non-naturally occurring amino acids, unless they are referred to by their full name, conventional abbreviations are employed according to the following table I, wherein the ‘*’ indicate the attachment points to other monomers of the compound to form the amide bonds. In case of any discrepancy in table I, the depicted formula is the truthful representation of the abbreviation.

TABLE I

| abbreviation | chemical name | formula |
|---|---|---|
| Tle | refers to the residue of L-tert- butylglycine (IUPAC name: (2S)-2-amino-3,3- dimethylbutanoic acid) | 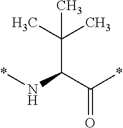 [SMILES: *N[C@H](C(*)=O)C(C)(C)C] |
| TrpMe | refers to the residue of 1-methyl-L- tryptophan (IUPAC name: (2S)-2-amino-3-(1- methyl-1H-indol-3-yl)propanoic acid) | 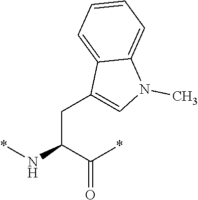 [SMILES: *N[C@@H](Cc1cn(C)c2ccccc12)C(*)=O] |
| bhAsn | refers to the residue of beta-homo-L- asparagine (IUPAC name: (3S)-3-amino-4- carbamoylbutanoic acid) | 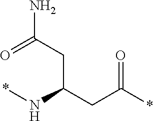 [SMILES: *N[C@H](CC(*)=O)CC(N)=O] |
| DprAc | refers to the residue of S-2-amino-3- acetamidopropanoic acid (IUPAC name: (2S)-2-amino-3- acetamidopropanoic acid) | 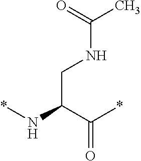 [SMILES: *N[C@@H](CNC(C)=O)C(*)=O] |
| Sar | refers to the residue of sarcosine (IUPAC name: 2-(methylamino)acetic acid) | 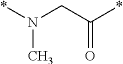 [SMILES: *C(=O)CN(*)C] |
| PEN | refers to the residue of L-penicillamine (IUPAC name: (2R)-2-amino-3-methyl- 3-sulfanylbutanoic acid) | 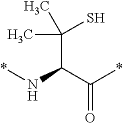 [SMILES: *N[C@H](C(*)=O)C(C)(C)S] |
| NMeI | refers to the residue of N-methyl-L- isoleucine (IUPAC name: (2S,3S)-3-methyl-2- (methylamino)pentanoic acid) | 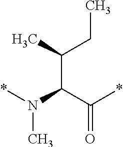 [SMILES: *C(=O)[C@H]([C@@H](C)CC)N(*)C] |
| NMeF | refers to the residue of N-methyl-L- phenylalanine (IUPAC name: (2S)-2-(methylamino)-3- phenylpropanic acid) | 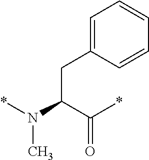 [SMILES: *C(=O)[C@H](Cc1ccccc1)N(*)C] |
| APCA | refers to the residue of 4-amino- piperidine-4 carboxylic acid (IUPAC name: 4-aminopiperidine-4- carboxylic acid) | 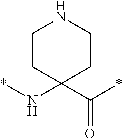 [SMILES: *NC1(C(*)=O)CCNCC1] |
| gGlu | refers to the residue of gamma-L- glutamate (IUPAC name: (2S)-2- aminopentanedioc acid) | 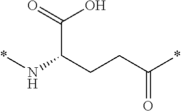 [SMILES: *N[C@@H](CCC(*)=O)C(=O)O] |

### Compstatin Analogue

The term “full length compstatin” as used herein refers to a 27 amino acid peptide having the sequence ICVVQDWGHHRCTAGHMANLTSHASAI (SEQ ID NO 103) wherein the cysteine residues at positions 2 and 12 are linked by a disulphide bond. The truncated form of such full length compstatin is set according to the sequence Hy-Ile(1)-Cys(2)-Val(3)-Val(4)-Gln(5)-Asp(6)-Trp(7)-Gly(8)-His(9)-His(10)-Arg(11)-Cys(12)-Thr(13)-NH₂ (SEQ ID NO 104) wherein the compound has a disulphide bridge between the side chains of the cysteine residues at positions 2 and 12 and retains the activity of the full length peptide; in present specification such truncated form including the above-mentioned thirteen amino acid residues is termed “compstatin”.

In the present context, the term “compstatin analogue” refers to a variant of such compstatin (i.e. SEQ ID NO 104) wherein one or more amino acid residues has been changed as compared to compstatin. These amino acid changes may represent, independently, one or more amino acid substitutions, additions, and/or deletions. The substitution of such one or more amino acids may be a substitution with natural amino acids as well as unnatural amino acids, including L- and D-stereoisomers thereof, as described in greater detail hereinafter.

The addition of such one or more amino acids may be an addition with natural amino acids as well as unnatural amino acids, including L- and D-stereoisomers thereof, as described in greater detail hereinafter.

In the present context, the position of an amino acid in the compstatin or in a compstatin analogue of the invention can be indicated as an integer number following the amino acid represented by an X (e.g. X0, X1, X2, etc.), or as an integer number in bracket after the amino acid conventional abbreviation (e.g. a isoleucine at position 1 can be indicated as I(1) or Ile(1)).

The compounds of the invention are represented by formula (I)

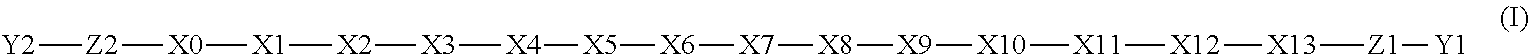

wherein the peptide portion from X1 to X13 (termed positions from one to thirteen) is the compstatin analogue portion corresponding to the thirteen amino acid residues of compstatin, X0 (termed position zero) is an additional amino acid residue linked to the N-terminus of X1 residue, Z1 (if present) is an n sequence of amino acid residues linked to the C-terminus of X13 residue, Z2 (if present) is an m sequence of amino acid residues linked to the N-terminus of X0 residue, Y1 is a terminal group present at the C-terminus of the peptide backbone, and Y2 is a terminal group present at the N-terminus of the peptide backbone (i.e. Y2 is bonded to the nitrogen atom of the N-terminal amino group and Y1 is bonded to the C-terminal carbonyl carbon atom of the peptide backbone).

Y2=hydrogen (also indicated as “Hy”) indicates a hydrogen atom, corresponding to the presence of a free primary or secondary amino group at the N-terminus of the peptide.

Y1=“OH” or “NH₂” indicates the presence of a carboxy (COOH) group or an amide (CONH₂) group at the C-terminus of the peptide compound, respectively. “A carboxy (COOH) group at the C-terminus of the peptide compound” means that the last amino acid residue at the C-terminus is in its natural form. When Z1 is absent, Y1=“K—OH” indicates the presence of an additional lysine residue, wherein the lysine is linked via its amino group to the carbonyl carbon atom of X13 and a carboxy (COOH) group at the C-terminus of such additional lysine is present (i.e. carboxy (COOH) group at the C-terminus of the peptide backbone).

The compounds of the invention of formula (I) or (Ia) are cyclized compounds by a disulphide bridge between the side chains of X2 and X12.

### Nomenclature of Compounds:

Compounds of the present invention are peptide chains built up of monomers (i.e. amino acid residues) wherein the amide bonds between two consequent monomers are represented by a hyphen “-”. The hyphen “-” is also used to indicate the link between the peptide backbone and the N-terminal or C-terminal group, respectively. Compounds of the present invention are all disulphide-cyclized compounds as defined in greater detail hereinafter. The connection of the two monomers that lead to the cyclization of a compound of the invention may be explicitly defined by a star in brackets “(*)” following each of the two monomers; such two monomers are cyclized by a disulphide bond (i.e. disulfide bond, —S—S—) formed by the respective side chains bearing each one a thiol group (—SH). Alternatively, can be described that X2 and X12 are linked by a disulphide bond.

As an example, the notation DTyr-I-C(*)-Tle-TrpMe-Q-D-W-Sar-A-H-R-PEN(*)-DArg-K—OH (compound No 1 in table A, SEQ ID NO: 1) means that cysteine residue C(*) at position 2 is linked to penicillamine residue PEN(*) at position 12 via the respective side chains by a disulphide bond (—S—S—) and completely defines the structure below depicted.

Moreover, when the terminal group present at the N-terminus of the peptide backbone is equal to hydrogen (i.e. Y2=Hy), such hydrogen is not explicitly represented in the specific sequence defining the specific compound of the invention; as an example, the notation DTyr-I-C(*)-Tle-TrpMe-Q-D-W-Sar-A-H-R-PEN(*)-DArg-K—OH (compound No 1 in table A, SEQ ID NO: 1) used in present specification corresponds to the sequence Hy-DTyr-I-C(*)-Tle-TrpMe-Q-D-W-Sar-A-H-R-PEN(*)-DArg-K—OH (SEQ ID NO: 1) wherein Y2=Hy is explicated.

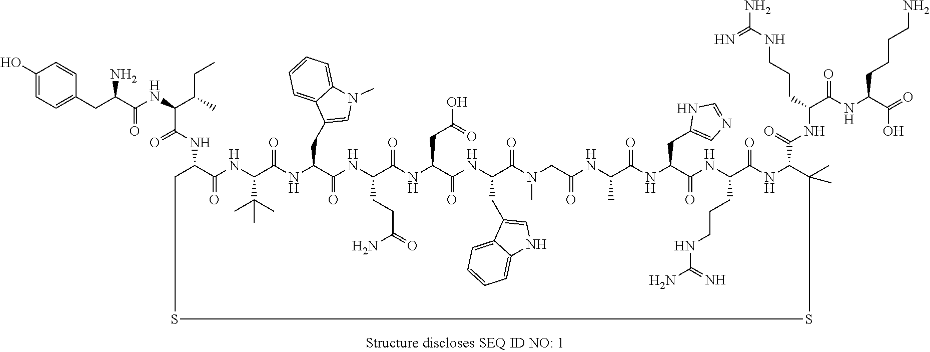 [SMILES: CC[C@H](C)[C@H](NC(=O)[C@H](N)Cc1ccc(O)cc1)C(=O)N[C@H]1CSSC(C)(C)[C@@H](C(=O)N[C@H](CCCNC(=N)N)C(=O)N[C@@H](CCCCN)C(=O)O)NC(=O)[C@H](CCCNC(=N)N)NC(=O)[C@H](Cc2cnc[nH]2)NC(=O)[C@H](C)NC(=O)CN(C)C(=O)[C@H](Cc2c[nH]c3ccccc23)NC(=O)[C@H](CC(=O)O)NC(=O)[C@H](CCC(N)=O)NC(=O)[C@H](Cc2cn(C)c3ccccc23)NC(=O)[C@H](C(C)(C)C)NC1=O]

### DETAILED DESCRIPTION OF THE INVENTION

In a first aspect, the invention provides a compound according to formula (I)

(I)
(SEQ ID NO: 105)
Y2-Z2-X0-X1-X2-X3-X4-X5-X6-X7-X8-X9-X10-X11-X12-X13-Z1-Y1

or salt thereof, preferably a pharmaceutically acceptable salt thereof,
wherein
X0 is DTyr,
X1 is I,
X2 is C,
X3 is Tle or V,
X4 is TrpMe,
X5 is selected from the group consisting of Q, DprAc, and bhAsn,
X6 is D,
X7 is W,
X8 is Sar,
X9 is A or E,
X10 is H,
X11 is R,
X12 is PEN,
X13 is selected from the group consisting of NMeI, DArg, DLys, DThr, DPhe, DTrp, P, DLeu, NMeF, and APCA, wherein the compound has a disulphide bridge between the sulphur atoms of X2 and X12 (i.e. formula (I) forming a cyclized structure),
and wherein
   (a) Z1 is a sequence of n amino acid residues independently selected from a group consisting of K, E, and gGlu, n is an integer selected from 1 to 4, Y1 is OH, Z2 is absent, and Y2 is hydrogen, with the proviso that when X13 is DTrp, Z1 is not a sequence of n K residues (SEQ ID NO: 108); or
   (b) Z1 is absent, Y1 is selected from the group consisting of OH, K—OH, and NH2, Z2 is a sequence of m amino acid residues independently selected from a group consisting of K, E, and gGlu, m is an integer selected from 1 to 4, and Y2 is hydrogen (SEQ ID NO: 109), with the proviso that when X13 is DTrp, Z2 is not a sequence of m K residues (SEQ ID NO: 110).

In a further aspect of formula (I), Z1 may be a sequence of n amino acid residues independently selected from a group consisting of the respectively D form of K, E, and gGlu, and Z2 may be a sequence of m amino acid residues independently selected from a group consisting of the respectively D form of K, E, and gGlu.

### Examples of Compounds of the Invention

TABLE A

| | | SEQ |
|---|---|---|
| Compound | Y2-Z2-X0-X1-X2-X3-X4-X5-X6-X7-X8-X9-X10-X11-X12-X13-Z1-Y1 | ID |
| No | having a disulphide bridge between the sulphur atoms of X2 and X12 | NO |
| 1 | DTyr-I-C(*)-Tle-TrpMe-Q-D-W-Sar-A-H-R-PEN(*)-DArg-K-OH | 1 |
| 2 | DTyr-I-C(*)-Tle-TrpMe-Q-D-W-Sar-A-H-R-PEN(*)-NMeI-K-OH | 2 |
| 3 | DTyr-I-C(*)-Tle-TrpMe-DprAc-D-W-Sar-A-H-R-PEN(*)-DArg-K-OH | 3 |
| 4 | DTyr-I-C(*)-Tle-TrpMe-Q-D-W-Sar-A-H-R-PEN(*)-NMeI-K-K-K-OH | 4 |
| 5 | DTyr-I-C(*)-Tle-TrpMe-Q-D-W-Sar-A-H-R-PEN(*)-NMeI-E-E-E-OH | 5 |
| 6 | DTyr-I-C(*)-Tle-TrpMe-Q-D-W-Sar-A-H-R-PEN(*)-NMeI-gGlu-gGlu-gGlu-OH | 6 |
| 7 | DTyr-I-C(*)-Tle-TrpMe-Q-D-W-Sar-A-H-R-PEN(*)-DThr-K-K-K-OH | 7 |
| 8 | DTyr-I-C(*)-Tle-TrpMe-Q-D-W-Sar-A-H-R-PEN(*)-DThr-E-E-E-OH | 8 |
| 9 | DTyr-I-C(*)-Tle-TrpMe-Q-D-W-Sar-A-H-R-PEN(*)-DThr-gGlu-gGlu-gGlu-OH | 9 |
| 10 | DTyr-I-C(*)-Tle-TrpMe-Q-D-W-Sar-A-H-R-PEN(*)-DArg-K-K-K-OH | 10 |
| 11 | DTyr-I-C(*)-Tle-TrpMe-Q-D-W-Sar-A-H-R-PEN(*)-DArg-E-E-E-OH | 11 |
| 12 | DTyr-I-C(*)-Tle-TrpMe-Q-D-W-Sar-A-H-R-PEN(*)-DArg-gGlu-gGlu-gGlu-OH | 12 |
| 13 | DTyr-I-C(*)-Tle-TrpMe-Q-D-W-Sar-A-H-R-PEN(*)-DLeu-K-K-K-OH | 13 |
| 14 | DTyr-I-C(*)-Tle-TrpMe-Q-D-W-Sar-A-H-R-PEN(*)-DLeu-E-E-E-OH | 14 |
| 15 | DTyr-I-C(*)-Tle-TrpMe-Q-D-W-Sar-A-H-R-PEN(*)-DLeu-gGlu-gGlu-gGlu-OH | 15 |
| 16 | DTyr-I-C(*)-Tle-TrpMe-Q-D-W-Sar-A-H-R-PEN(*)-DTrp-E-E-E-OH | 16 |
| 17 | DTyr-I-C(*)-Tle-TrpMe-Q-D-W-Sar-A-H-R-PEN(*)-DTrp-gGlu-gGlu-gGlu-OH | 17 |
| 18 | DTyr-I-C(*)-Tle-TrpMe-Q-D-W-Sar-A-H-R-PEN(*)-NMeI-K-gGlu-gGlu-gGlu-OH | 18 |
| 19 | DTyr-I-C(*)-Tle-TrpMe-Q-D-W-Sar-A-H-R-PEN(*)-DTrp-K-gGlu-gGlu-gGlu-OH | 19 |
| 20 | K-K-K-DTyr-I-C(*)-Tle-TrpMe-Q-D-W-Sar-A-H-R-PEN(*)-NMeI-NH2 | 20 |
| 21 | E-E-E-DTyr-I-C(*)-Tle-TrpMe-Q-D-W-Sar-A-H-R-PEN(*)-NMeI-NH2 | 21 |
| 22 | gGlu-gGlu-gGlu-DTyr-I-C(*)-Tle-TrpMe-Q-D-W-Sar-A-H-R-PEN(*)-NMeI-NH₂ | 22 |
| 23 | K-K-K-DTyr-I-C(*)-Tle-TrpMe-Q-D-W-Sar-A-H-R-PEN(*)-DThr-NH₂ | 23 |
| 24 | E-E-E-DTyr-I-C(*)-Tle-TrpMe-Q-D-W-Sar-A-H-R-PEN(*)-DThr-NH₂ | 24 |
| 25 | gGlu-Glu-gGlu-DTyr-I-C(*)-Tle-TrpMe-Q-D-W-Sar-A-H-R-PEN(*)-DThr-NH₂ | 25 |
| 26 | K-K-K-DTyr-I-C(*)-Tle-TrpMe-Q-D-W-Sar-A-H-R-PEN(*)-DArg-NH₂ | 26 |
| 27 | E-E-E-DTyr-I-C(*)-Tle-TrpMe-Q-D-W-Sar-A-H-R-PEN(*)-DArg-NH₂ | 27 |
| 28 | gGlu-gGlu-gGlu-DTyr-I-C(*)-Tle-TrpMe-Q-D-W-Sar-A-H-R-PEN(*)-DArg-NH₂ | 28 |
| 29 | K-K-K-DTyr-I-C(*)-Tle-TrpMe-Q-D-W-Sar-A-H-R-PEN(*)-DLeu-NH₂ | 29 |
| 30 | E-E-E-DTyr-I-C(*)-Tle-TrpMe-Q-D-W-Sar-A-H-R-PEN(*)-DLeu-NH₂ | 30 |
| 31 | gGlu-gGlu-gGlu-DTyr-I-C(*)-Tle-TrpMe-Q-D-W-Sar-A-H-R-PEN(*)-DLeu-NH₂ | 31 |
| 32 | E-E-E-DTyr-I-C(*)-Tle-TrpMe-Q-D-W-Sar-A-H-R-PEN(*)-DTrp-NH₂ | 32 |
| 33 | gGlu-gGlu-gGlu-DTyr-I-C(*)-Tle-TrpMe-Q-D-W-Sar-A-H-R-PEN(*)-DTrp-NH₂ | 33 |
| 34 | gGlu-gGlu-gGlu-DTyr-I-C(*)-Tle-TrpMe-Q-D-W-Sar-A-H-R-PEN(*)-NMeI-K-OH | 34 |
| 35 | gGlu-gGlu-gGlu-DTyr-I-C(*)-Tle-TrpMe-Q-D-W-Sar-A-H-R-PEN(*)-DTrp-K-OH | 35 |
| 36 | DTyr-I-C(*)-V-TrpMe-Q-D-W-Sar-A-H-R-PEN(*)-NMeI-K-K-K-OH | 36 |
| 37 | DTyr-I-C(*)-V-TrpMe-Q-D-W-Sar-A-H-R-PEN(*)-NMeI-gGlu-gGlu-gGlu-OH | 37 |
| 38 | DTyr-I-C(*)-V-TrpMe-Q-D-W-Sar-A-H-R-PEN(*)-DThr-K-K-K-OH | 38 |
| 39 | DTyr-I-C(*)-V-TrpMe-Q-D-W-Sar-A-H-R-PEN(*)-DThr-gGlu-gGlu-gGlu-OH | 39 |
| 40 | DTyr-I-C(*)-Tle-TrpMe-Q-D-W-Sar-E-H-R-PEN(*)-NMeI-gGlu-gGlu-gGlu-OH | 40 |
| 41 | DTyr-I-C(*)-Tle-TrpMe-Q-D-W-Sar-E-H-R-PEN(*)-NMeI-K-K-K-OH | 41 |
| 42 | DTyr-I-C(*)-Tle-TrpMe-Q-D-W-Sar-E-H-R-PEN(*)-DThr-gGlu-gGlu-gGlu-OH | 42 |
| 43 | DTyr-I-C(*)-Tle-TrpMe-Q-D-W-Sar-E-H-R-PEN(*)-DThr-K-K-K-OH | 43 |
| 44 | DTyr-I-C(*)-Tle-TrpMe-bhAsn-D-W-Sar-A-H-R-PEN(*)-DThr-gGlu-gGlu-gGlu-OH | 44 |
| 45 | DTyr-I-C(*)-Tle-TrpMe-Q-D-W-Sar-A-H-R-PEN(*)-NMeF-gGlu-gGlu-gGlu-OH | 45 |
| 46 | DTyr-I-C(*)-Tle-TrpMe-Q-D-W-Sar-A-H-R-PEN(*)-P-gGlu-gGlu-gGlu-OH | 46 |
| 47 | DTyr-I-C(*)-Tle-TrpMe-Q-D-W-Sar-A-H-R-PEN(*)-DLys-gGlu-gGlu-Glu-OH | 47 |
| 48 | DTyr-I-C(*)-Tle-TrpMe-Q-D-W-Sar-A-H-R-PEN(*)-DLys-K-K-K-OH | 48 |
| 49 | DTyr-I-C(*)-Tle-TrpMe-Q-D-W-Sar-A-H-R-PEN(*)-DPhe-gGlu-gGlu-gGlu-OH | 49 |
| 50 | DTyr-I-C(*)-Tle-TrpMe-DprAc-D-W-Sar-A-H-R-PEN(*)-DArg-K-K-K-OH | 50 |
| 51 | DTyr-I-C(*)-Tle-TrpMe-DprAc-D-W-Sar-A-H-R-PEN(*)-DArg-gGlu-gGlu-gGlu-OH | 51 |
| 52 | DTyr-I-C(*)-Tle-TrpMe-DprAc-D-W-Sar-A-H-R-PEN(*)-NMeI-K-K-K-OH | 52 |
| 53 | DTyr-I-C(*)-Tle-TrpMe-DprAc-D-W-Sar-A-H-R-PEN(*)-DThr-gGlu-gGlu-gGlu-OH | 53 |
| 54 | DTyr-I-C(*)-Tle-TrpMe-DprAc-D-W-Sar-E-H-R-PEN(*)-NMeI-K-K-K-OH | 54 |
| 55 | DTyr-I-C(*)-Tle-TrpMe-Q-D-W-Sar-A-H-R-PEN(*)-DPhe-K-K-K-OH | 55 |
| 56 | DTyr-I-C(*)-Tle-TrpMe-Q-D-W-Sar-A-H-R-PEN(*)-P-K-K-K-OH | 56 |
| 57 | DTyr-I-C(*)-Tle-TrpMe-Q-D-W-Sar-A-H-R-PEN(*)-NMeF-K-K-K-OH | 57 |
| 58 | DTyr-I-C(*)-Tle-TrpMe-Q-D-W-Sar-E-H-R-PEN(*)-DLys-K-K-K-OH | 58 |
| 59 | DTyr-I-C(*)-Tle-TrpMe-Q-D-W-Sar-E-H-R-PEN(*)-P-K-K-K-OH | 59 |
| 60 | DTyr-I-C(*)-Tle-TrpMe-Q-D-W-Sar-E-H-R-PEN(*)-NMeF-K-K-K-OH | 60 |
| 61 | DTyr-I-C(*)-Tle-TrpMe-Q-D-W-Sar-E-H-R-PEN(*)-DArg-K-K-K-OH | 61 |
| 62 | DTyr-I-C(*)-Tle-TrpMe-Q-D-W-Sar-E-H-R-PEN(*)-DPhe-K-K-K-OH | 62 |
| 63 | DTyr-I-C(*)-Tle-TrpMe-DprAc-D-W-Sar-A-H-R-PEN(*)-DThr-K-K-K-OH | 63 |
| 64 | DTyr-I-C(*)-Tle-TrpMe-DprAc-D-W-Sar-A-H-R-PEN(*)-DLys-K-K-K-OH | 64 |
| 66 | DTyr-I-C(*)-Tle-TrpMe-DprAc-D-W-Sar-A-H-R-PEN(*)-NMeF-K-K-K-OH | 66 |
| 67 | DTyr-I-C(*)-Tle-TrpMe-DprAc-D-W-Sar-A-H-R-PEN(*)-DPhe-K-K-K-OH | 67 |
| 68 | DTyr-I-C(*)-Tle-TrpMe-DprAc-D-W-Sar-E-H-R-PEN(*)-DThr-K-K-K-OH | 68 |
| 69 | DTyr-I-C(*)-Tle-TrpMe-DprAc-D-W-Sar-E-H-R-PEN(*)-DLys-K-K-K-OH | 69 |
| 70 | DTyr-I-C(*)-Tle-TrpMe-DprAc-D-W-Sar-E-H-R-PEN(*)-P-K-K-K-OH | 70 |
| 71 | DTyr-I-C(*)-Tle-TrpMe-DprAc-D-W-Sar-E-H-R-PEN(*)-DPhe-K-K-K-OH | 71 |
| 72 | DTyr-I-C(*)-Tle-TrpMe-bhAsn-D-W-Sar-A-H-R-PEN(*)-DThr-K-K-K-OH | 72 |
| 73 | DTyr-I-C(*)-Tle-TrpMe-bhAsn-D-W-Sar-A-H-R-PEN(*)-DLys-K-K-K-OH | 73 |
| 74 | DTyr-I-C(*)-Tle-TrpMe-bhAsn-D-W-Sar-A-H-R-PEN(*)-P-K-K-K-OH | 74 |
| 75 | DTyr-I-C(*)-Tle-TrpMe-bhAsn-D-W-Sar-A-H-R-PEN(*)-DArg-K-K-K-OH | 75 |
| 76 | DTyr-I-C(*)-Tle-TrpMe-bhAsn-D-W-Sar-A-H-R-PEN(*)-DPhe-K-K-K-OH | 76 |
| 77 | DTyr-I-C(*)-Tle-TrpMe-bhAsn-D-W-Sar-E-H-R-PEN(*)-DThr-K-K-K-OH | 77 |
| 78 | DTyr-I-C(*)-Tle-TrpMe-bhAsn-D-W-Sar-E-H-R-PEN(*)-DLys-K-K-K-OH | 78 |
| 79 | DTyr-I-C(*)-Tle-TrpMe-bhAsn-D-W-Sar-E-H-R-PEN(*)-APCA-K-K-K-OH | 79 |
| 80 | DTyr-I-C(*)-Tle-TrpMe-bhAsn-D-W-Sar-E-H-R-PEN(*)-DPhe-K-K-K-OH | 80 |
| 81 | K-K-K-DTyr-I-C(*)-Tle-TrpMe-DprAc-D-W-Sar-A-H-R-PEN(*)-DArg-OH | 81 |
| 82 | K-K-K-DTyr-I-C(*)-Tle-TrpMe-DprAc-D-W-Sar-A-H-R-PEN(*)-DThr-OH | 82 |
| 83 | K-K-K-DTyr-I-C(*)-Tle-TrpMe-DprAc-D-W-Sar-A-H-R-PEN(*)-DLys-OH | 83 |
| 84 | K-K-K-DTyr-I-C(*)-Tle-TrpMe-DprAc-D-W-Sar-A-H-R-PEN(*)-P-OH | 84 |
| 85 | K-K-K-DTyr-I-C(*)-Tle-TrpMe-DprAc-D-W-Sar-A-H-R-PEN(*)-DPhe-OH | 85 |
| 86 | K-K-K-DTyr-I-C(*)-Tle-TrpMe-DprAc-D-W-Sar-E-H-R-PEN(*)-DArg-OH | 86 |
| 87 | K-K-K-DTyr-I-C(*)-Tle-TrpMe-DprAc-D-W-Sar-E-H-R-PEN(*)-DThr-OH | 87 |
| 88 | K-K-K-DTyr-I-C(*)-Tle-TrpMe-DprAc-D-W-Sar-E-H-R-PEN(*)-DLys-OH | 88 |
| 89 | K-K-K-DTyr-I-C(*)-Tle-TrpMe-bhAsn-D-W-Sar-E-H-R-PEN(*)-DArg-OH | 89 |
| 90 | K-K-K-DTyr-I-C(*)-Tle-TrpMe-bhAsn-D-W-Sar-E-H-R-PEN(*)-DThr-OH | 90 |
| 91 | K-K-K-DTyr-I-C(*)-Tle-TrpMe-bhAsn-D-W-Sar-E-H-R-PEN(*)-DLys-OH | 91 |
| 92 | K-K-K-DTyr-I-C(*)-Tle-TrpMe-bhAsn-D-W-Sar-E-H-R-PEN(*)-P-OH | 92 |
| 93 | K-K-K-DTyr-I-C(*)-Tle-TrpMe-bhAsn-D-W-Sar-E-H-R-PEN(*)-DPhe-OH | 93 |
| 94 | DTyr-I-C(*)-Tle-TrpMe-Q-D-W-Sar-A-H-R-PEN(*)-DThr-DLys-DLys-DLys-OH | 94 |
| 95 | DTyr-I-C(*)-Tle-TrpMe-bhAsn-D-W-Sar-A-H-R-PEN(*)-DThr-DgGlu-DgGlu-DgGlu-OH | 95 |
| 96 | DTyr-I-C(*)-Tle-TrpMe-bhAsn-D-W-Sar-A-H-R-PEN(*)-DLys-DLys-DLys-DLys-OH | 96 |
| 97 | DTyr-I-C(*)-Tle-TrpMe-DprAc-D-W-Sar-E-H-R-PEN(*)-DThr-DLys-DLys-DLys-OH | 97 |
| 98 | DTyr-I-C(*)-Tle-TrpMe-bhAsn-D-W-Sar-A-H-R-PEN(*)-DThr-gGlu-gGlu-OH | 98 |
| 99 | DTyr-I-C(*)-Tle-TrpMe-bhAsn-D-W-Sar-A-H-R-PEN(*)-DThr-gGlu-OH | 99 |
| 100 | DTyr-I-C(*)-Tle-TrpMe-bhAsn-D-W-Sar-A-H-R-PEN(*)-DLys-K-K-OH | 100 |
| 101 | DTyr-I-C(*)-Tle-TrpMe-bhAsn-D-W-Sar-A-H-R-PEN(*)-DLys-K-OH | 113 |

wherein C(*) and PEN(*) indicate the pairs of amino acid residues X2 and X12 bridged between the sulphur atoms of their side chains, forming a disulphide bond; and Y2 is equal to hydrogen in all compounds and not represented.

### Compounds No Part of the Invention

TABLE A.1

| | | SEQ |
|---|---|---|
| Compound | | ID |
| No | | NOs |
| 102 | DTyr-I-C(*)-Tle-TrpMe-bhAsn-D-W-Sar-A-H-R-PEN(*)-DThr-OH | 114 |
| 103 | DTyr-I-C(*)-Tle-TrpMe-bhAsn-D-W-Sar-A-H-R-PEN(*)-DLys-OH | 115 |

wherein C(*) and PEN(*) indicate the pairs of amino acid residues X2 and X12 bridged between the sulphur atoms of their side chains, forming a disulphide bond.

### Reference Compounds

TABLE B

| Reference | | SEQ |
|---|---|---|
| Compound | | ID |
| No | | NOs |
| Ref.1# | DTyr-I-C(*)-V-TrpMe-Q-D-W-Sar-A-H-R-C(*)-NMeI-NH2 | 101 |
| Ref.2## | DTyr-I-C(*)-V-TrpMe-Q-D-W-Sar-A-H-R-C(*)-NMeI-K-K-K-NH2 | 102 |
| Ref.3### | Ac-I-C(*)-V-TrpMe-Q-D-W-G-A-H-R-PEN(*)-T-NH2 | 116 |

#Ref. 1 is SEQ ID NO 7 of WO2019/195712 A1;
##Ref. 2 is SEQ ID NO 10 of WO2019/195712 A1.
###Ref. 3 is SEQ ID NO: 217 in CN118834265 A. Both Ref.1, Ref.2 and Ref.3 are cyclised peptides, wherein the compounds have a disulphide bridge between the sulphur atoms of the two cysteine residues C(*).

Reference compound Ref. 1, that binds C3 protein inhibiting complement activation, shows good chemical stability but low solubility at or around physiological pH, as disclosed in Table 1 to 3 below.

Reference compound Ref. 2, that binds C3 protein inhibiting complement activation, shows good solubility but low chemical stability at or around physiological pH, as disclosed in Table 1 to 3 below.

Reference compound Ref. 3 binds C3 protein, inhibiting complement activation, with reduced affinity (i.e. KD 16.30 nM) with respect to the compounds of the invention, shows low solubility and moderate chemical stability at or around physiological pH, as disclosed in Tables 1 to 3 hereinafter.

On the other hand, compounds according to the invention not only bind C3 protein inhibiting complement activation with high affinity and potency but show both high solubility and high chemical stability at or around physiological pH, as disclosed in Table 1 to 3 below, for being viable for human treatment, particularly for treatment by intravitreal administration, for example IVT injection. In fact, the compounds according to the invention have increased solubility at or around physiological pH with respect to reference compound Ref. 1 and increased chemical stability at or around physiological pH with respect to reference compound Ref. 2, as disclosed in Table 2 and 3 below.

The compounds of the invention according to formula (I) or (Ia), and any embodiment disclosed herewith, bind to C3 protein and inhibit complement activation (i.e. inhibit complement factor C3), see table 1. The skilled person will be aware of suitable assay formats, and an example is provided hereinafter. For instance, for compounds of the present invention the functional potency to C3 protein is evaluated by the assays as described in Example 1A and Example 1B. The affinity for compounds of the present invention to the C3 protein is evaluated by surface plasmon resonance (SPR), as described in Example 1C, and high affinity to C3 is considered to be a key factor for high biological activity. The compounds of the present invention show an affinity towards the C3 protein at KD's below 100 nM (e.g. 100 or 50 nM to 0.005 nM). In a preferred embodiment of compounds of the present invention, the KD towards the C3 protein is below 10 nM (e.g. 10 or 5 nM to 0.01 nM). In a highly preferred embodiment of compounds of the present invention, the EC₅₀ towards C3 protein is below or equal to 1 nM (e.g. 1 nM to 0.01 nM).

The compounds of the invention according to formula (I) or (Ia) are soluble at or around physiological pH (see table 2) and therefore suitable for use in human therapy by parenteral administration, preferably by intravitreal administration (e.g. intravitreal injection). In the context of the present invention the term “at or around physiological pH” means at or around pH 7.4, e.g. pH 7.4±0.5. There are several techniques known to the skilled person in the art how to determine solubility. Preferably the solubility of the peptide of the invention is determined as disclosed in Example 2 hereinafter. Generally, the solubility of the compounds of the invention is greater than or equal to 4.0 mg/ml (±0.5) at or around physiological pH. In a preferred embodiment, the solubility of the compounds of the invention is greater than or equal to 5.0 mg/ml (±0.5) at or around physiological pH.

The compounds of the invention according to formula (I) or (Ia) have favourable chemical stability at or around physiological pH, being suitable for use in human therapy by parenteral administration, preferably by intravitreal administration (e.g. intravitreal injection). In this regard, preferably the chemical degradation of the compounds of the invention at or around physiological pH after 14 days at 40° C. is lower than or equal to 2% (±0.5), or lower than or equal to 1% (±0.5), or not detectable (measurement described in Example 3 and table 3).

In one embodiment of the compounds of the present invention, the affinity towards the C3 protein is at KD less than 10 nM, the solubility is greater than or equal to 4.0 mg/ml (±0.5) at or around physiological pH, and the chemical degradation of the compounds of the invention at or around physiological pH after 14 days at 40° C. may be lower than or equal to 2% (±0.5), according to the methods described in the experimental part. In a preferred embodiment of the compounds of the present invention, the affinity towards the C3 protein is at KD less than 1 nM, the solubility is greater than or equal to 5.0 mg/ml (±0.5) at or around physiological pH, and the chemical degradation of the compounds of the invention at or around physiological pH after 14 days at 40° C. may be lower than or equal to 2% (t 0.5).

### Pharmaceutical Compositions and Administration

In a second aspect, the invention provides pharmaceutical compositions (i.e. formulation) comprising at least one compound of formula (I), (Ia) or a pharmaceutically acceptable salt thereof, and at least one pharmaceutically acceptable carrier and/or excipient.

According to the present invention, the compound of the invention may be in the form of a pharmaceutically acceptable salt. Pharmaceutically acceptable salts are intended to include any salts that are commonly used in formulations of peptides or derivative thereof. Such salts include both acid addition salts and basic salts. Suitable salts formed with bases include metal salts, such as alkali metal or alkaline earth metal salts. In one embodiment, a pharmaceutical composition is one wherein the compound of the invention is in the form of a pharmaceutically acceptable acid addition salt, such as hydrochloride or acetate.

The term “pharmaceutically acceptable carrier and/or excipient” includes any of the standard pharmaceutical carrier and excipient well known in the pharmaceutical art and commonly used in formulations of peptides or derivative thereof. The term further encompasses any carrier and/or excipients agents suitable for peptides formulation and listed in the US Pharmacopeia for use in humans.

Pharmaceutical compositions of the present invention are suitable for use in a method of preventing and/or treating diseases or disorders as disclosed hereinafter. Said pharmaceutical compositions, which are suited for administration to a human being in the need thereof, typically comprise a therapeutically effective amount of the active ingredient. The actual pharmaceutically effective amount or therapeutic dosage will usually depend on factors known by those skilled in the art such as age and physical condition of the patient, route of administration and severity of disease. In any case, the compounds will be administered at dosages and in a manner, which allows a pharmaceutically effective amount to be delivered based upon patient's unique condition and the knowledge of the physician.

It should be appreciated that different formulations may be used depending on physicochemical characteristic of the compound of the invention. It should be appreciated that different routes of administration may be used depending on the choice of formulation and chemical and/or metabolic stability of the compound of invention. Such administration routes may include but are not limited to parenteral administration, such as intravitreal (IVT), intravenous (IV), subcutaneous (SC), intradermal (ID), or intramuscular (IM). In a preferred embodiment of the invention, the administration route is an intravitreal administration, most preferably intravitreal injection.

Different pharmaceutical form may be used depending on the formulation and administration route. In a preferred aspect of the invention, the pharmaceutical form is a solution for parenteral administration, preferably for intravitreal administration, most preferably intravitreal injection. Alternatively, the pharmaceutical form may be a suspension or gel for parenteral administration, preferably for intravitreal administration, most preferably intravitreal injection.

The human dose range of a compound according to formula (I), (Ia) or a pharmaceutically acceptable salt thereof, alone or as part of a pharmaceutical composition, applicable by administration to an individual in the need thereof by intravitreal administration (e.g. IVT injection) is usually from 0.001 μg/Kg to 400 μg/Kg body weight when administered once monthly, once every two months, once every three months, once every six months, once every several months or once every twelve months.

The human dose range of a compound according to formula (I), (Ia) or a pharmaceutically acceptable salt thereof, alone or as part of a pharmaceutical composition, applicable by administration to an individual in the need thereof by parenteral administration is usually from 0.01 mg/Kg to 20 mg/Kg body weight when administered once monthly, once every two months, once every three months, once every six months, once every several months or once every twelve months.

Particularly, the compound of the invention according to formula (I) or (Ia) are potent inhibitor of complement factor C3, are both soluble and chemically stable at or around physiological pH to be suitable for intravitreal administration (e.g. intravitreal injections) to humans administered at long interval of time between two consecutive injections, preferably for intravitreal injection administration once monthly, or even better, once every two months, once every three months, once every six months, once every several months or once every twelve months.

### Method of Treatment/Use in Treatment

In a third aspect, the invention provides compounds according to formula (I), (Ia) or pharmaceutical acceptable salts thereof, according to any of the aspects and embodiments disclosed herein, for use as a medicament.

Particularly, the invention provides compounds according to formula (I), (Ia) or pharmaceutical acceptable salts thereof for use in a method of inhibiting complement activation. By way of example, inhibiting complement activation includes one or more biological activities selected from (1) binding to C3 protein, (2) binding to C3b protein and/or (3) inhibiting the cleavage of native C3 by C3 convertases.

In a further aspect, described herein are compounds according to formula (I) or (Ia) for use in a method of inhibiting complement activation that occurs during cell or organ transplantation.

Excessive activation or inappropriate regulation of complement can lead to a number of pathologic conditions, ranging from autoimmune diseases to inflammatory diseases, as well described in literature.

Complement protein C3 is a central component of the complement system and acts as the convergence point for all three complement activation pathways: classical, lectin, and alternative. Once C3 is cleaved into C3a and C3b, it amplifies the complement cascade by promoting opsonization, inflammation, and formation of the C5 convertase, which ultimately leads to the formation of the cell damaging membrane attack complex (MAC). Inhibiting C3 will effectively block this critical amplification step, preventing downstream activation regardless of which pathway initiated the response. This makes C3 inhibition a broad approach for treating diseases driven by excessive or insufficiently controlled complement activity.

The use of a C3 inhibitor for treating or preventing an eye or ocular disease or other diseases is known in the art. For example, SYFOVRE® (pegcetacoplan), a C3 inhibitor, is approved by FDA for the treatment of geographic atrophy (GA), and Empaveli® (pegcetacoplan) for the treatment of paroxysmal nocturnal haemoglobinuria (PNH), C3 glomerulopathy (C3G) or primary immune-complex membranoproliferative glomerulonephritis (IC-MPGN), to reduce proteinuria.

In a preferred embodiment the invention provides compounds according to formula (I), (Ia) or pharmaceutical acceptable salts thereof, according to any of the aspects and embodiments disclosed herein, for use in a method of treating or preventing an eye or ocular disease in a patient in need thereof. Said eye or ocular disease is selected from the group consisting of retinopathy, proliferative retinopathy (PR) such as retinopathy of prematurity, ischemic retinopathy, diabetic retinopathy (DR) including proliferative diabetic retinopathy (PDR) and non-proliferative diabetic retinopathy, diabetic macular edema (DME), diabetic macular ischemia (DMI), age-related macular degeneration (AMD) including dry AMD and wet AMD, geographic atrophy (GA), retinitis pigmentosa, inherited retinal dystrophy, myopic degeneration, retinal vein occlusions, retinal artery occlusions, endophthalmitis, uveitis, cystoid macular edema, choroidal neovascular membrane secondary to any retinal diseases, optic neuropathies, glaucoma, neovascular glaucoma (NVG), retinal detachment, toxic retinopathy, radiation retinopathy, traumatic retinopathy, drug-induced retinal vasculopathy, retinal neovascularisation, polypoidal choroidal vasculopathy, retinal vasculitis, retinal microaneurysm, retrolental fibroplasia, chorioretinitis, Fuch's dystrophy, macular telangiectasia, Usher syndrome, Stargardt disease, and neuromyelitis optica spectrum disorder. In a preferred embodiment, the invention encompasses compounds of formula (I), (Ia) or pharmaceutical acceptable salts thereof for use in the treatment or prevention of a disease selected from the group consisting of age-related macular degeneration (AMD) including dry AMD and wet AMD, and geographic atrophy (GA), neovascular glaucoma (NVG), and diabetic retinopathy, most preferably geographic atrophy (GA).

In a further embodiment the invention provides compounds according to formula (I), (Ia) or pharmaceutical acceptable salts thereof, according to any of the aspects and embodiments disclosed herein, for use in a method of treating or preventing a disease in a patient in need thereof selected from:
periodontitis, rheumatoid arthritis, spinal cord injury, stroke, multiple sclerosis;
Parkinson's disease, Alzheimer's disease; cancer;
respiratory disorders such as asthma, chronic obstructive pulmonary disease (COPD), allergic inflammation, emphysema, bronchitis, bronchiecstasis, cystic fibrosis, tuberculosis, pneumonia, respiratory distress syndrome (RDS—neonatal and adult), rhinitis and sinusitis;
bacterial infections such as sepsis, ischemia-reperfusion injury in various tissues, myocardial infarction, anaphylaxis, paroxysmal nocturnal hemoglobinuria (PNH), autoimmune hemolytic anemias, psoriasis, hidradentitis suppurativa, myasthenia gravis, systemic lupus erythematosus, CHAPLE syndrome, C3 glomeropathy (C3G), IgA nephropathy, atypical hemolytic uremic syndrome, Crohn's disease, ulcerative colitis, antiphospholipid syndrome, endometriosis, systemic sclerosis, and primary immune-complex membranoproliferative glomerulonephritis (IC-MPGN).

In a fourth aspect, the invention provides a method of inhibiting complement activation for treating a subject in need thereof, particularly provides any method for treating or preventing disease listed hereinbefore in a patient in need thereof, preferably an eye or ocular diseases, most preferably geographic atrophy (GA), said method comprising the administration of an effective amount of a compound of the invention according to formula (I) or a pharmaceutical acceptable salt thereof.

Likewise, the present invention relates to the use of compounds of formula (I), (Ia) or pharmaceutically acceptable salts thereof in the manufacture of a medicament for inhibiting complement activation and/or for the treatment of a disease listed hereinbefore, according to any of the aspects and embodiments disclosed herein.

The terms “treatment” and grammatical variants thereof as employed in the present context refer to an approach for obtaining beneficial or desired clinical results. For the purposes of this invention, beneficial or desired clinical results include, but are not limited to, alleviation of symptoms, diminishment of extent of disease, stabilization (i.e. not worsening) of state of disease, delay or slowing of disease progression, amelioration or palliation of the disease state, and remission (whether partial or total), whether detectable or undetectable. The term “treatment” includes inhibition or reduction of an increase in severity of a pathological state or symptoms relative to the absence of treatment, and is not necessarily meant to imply complete cessation of the relevant disease, disorder or condition. The terms “prevention” and grammatical variants thereof as employed in the present context refer to an approach for hindering or preventing the development of, or altering the pathology of, a condition, disease, or disorder. Accordingly, “prevention” may refer to prophylactic or preventive measures. For the purposes of this invention, beneficial or desired clinical results include, but are not limited to, prevention or slowing of symptoms, progression, or development of a disease, whether detectable or undetectable. A subject in need of “prevention” may thus be a subject not yet afflicted with the disease or disorder in question. The term “prevention” thus includes inhibiting or slowing the onset of disease relative to the absence of treatment and is not necessarily meant to imply permanent prevention of the relevant disease, disorder, or condition. The terms “patient”, “subject,” and “individual” may be used interchangeably and refer to a human.

### Embodiments (Abbreviated as Emb.) of the Present Invention

### Embodiments 1 to 52

Emb. 1. A compound according to formula (I)

(I)
(SEQ ID NO: 105)
Y2-Z2-X0-X1-X2-X3-X4-X5-X6-X7-X8-X9-X10-X11-X12-X13-Z1-Y1

or salt thereof, preferably a pharmaceutically acceptable salt thereof,
wherein
X0 is DTyr,
X1 is I,
X2 is C,
X3 is Tle or V,
X4 is TrpMe,
X5 is selected from the group consisting of Q, DprAc, and bhAsn,
X6 is D,
X7 is W,
X8 is Sar,
X9 is A or E,
X10 is H,
X11 is R,
X12 is PEN,
X13 is selected from the group consisting of NMeI, DArg, DLys, DThr, DPhe, DTrp, P, DLeu, NMeF, and APCA, wherein the compound has a disulphide bridge between the sulphur atoms of X2 and X12,
and wherein
   (a) Z1 is a sequence of n amino acid residues independently selected from a group consisting of K, E, and gGlu, n is an integer selected from 1 to 4, Y1 is OH, Z2 is absent, and Y2 is hydrogen, with the proviso that when X13 is DTrp, Z1 is not a sequence of n K residues (SEQ ID NO: 108); or
   (b) Z1 is absent, Y1 is selected from the group consisting of OH, K—OH, and NH2, Z2 is a sequence of m amino acid residues independently selected from a group consisting of K, E, and gGlu, m is an integer selected from 1 to 4, and Y2 is hydrogen (SEQ ID NO: 109), with the proviso that when X13 is DTrp, Z2 is not a sequence of m K residues (SEQ ID NO: 110);
[i.e. Hy-Z2-DTyr-I-C(*)-X3-TrpMe-X5-D-W-Sar-X9-H-R-PEN(*)-X13-Z1-Y1 (SEQ ID NO: 105), wherein X3, X5, X9, X13, Z1 and Z2 are as defined in Emb. 1, and wherein the side chains of C(*) and PEN(*) are linked by a disulphide bond].

Emb. 1a. The compound according to embodiment 1, wherein
(a) Z1 is a sequence of n amino acid residues independently selected from a group consisting of K, E, and gGlu, n is an integer selected from 1 to 4, Y1 is OH, Z2 is absent, and Y2 is hydrogen, with the proviso that when X13 is DTrp, Z1 is not a sequence of n K residues.

Emb. 1b. The compound according to embodiment 1, wherein
(b) Z1 is absent, Y1 is selected from the group consisting of OH, K—OH, and NH2, Z2 is a sequence of m amino acid residues independently selected from a group consisting of K, E, and gGlu, m is an integer selected from 1 to 4, and Y2 is hydrogen, with the proviso that when X13 is DTrp, Z2 is not a sequence of m K residues.

Emb. 1c. The compound according to embodiment 1, wherein
Z1 may be a sequence of n amino acid residues independently selected from a group consisting of the respectively D form of K, E, and gGlu, and Z2 may be a sequence of m amino acid residues independently selected from a group consisting of the respectively D form of K, E, and gGlu.

Emb. 2. A compound according to formula (II)

(II)
(SEQ ID NO: 106)
Y2-X0-X1-X2-X3-X4-X5-X6-X7-X8-X9-X10-X11-X12-X13-Z1-Y1;

or salt thereof, preferably a pharmaceutically acceptable salt thereof,
wherein
X0 is DTyr,
X1 is I,
X2 is C,
X3 is Tle or V,
X4 is TrpMe,
X5 is selected from the group consisting of Q, DprAc, and bhAsn,
X6 is D,
X7 is W,
X8 is Sar,
X9 is A or E,
X10 is H,
X11 is R,
X12 is PEN,
X13 is selected from the group consisting of NMeI, DArg, DLys, DThr, DPhe, DTrp, P, DLeu, NMeF, and APCA,
wherein the compound has a disulphide bridge between the sulphur atoms of X2 and X12,
and wherein
   Z1 is a sequence of n amino acid residues independently selected from the group consisting of K, E, and gGlu, n is an integer selected from 1 to 4, Y1 is OH, and Y2 is hydrogen, with the proviso that when X13 is DTrp, Z1 is not a sequence of n K residues (SEQ ID NO: 111);
[i.e. Hy-DTyr-I-C(*)-X3-TrpMe-X5-D-W-Sar-X9-H-R-PEN(*)-X13-Z1-OH (SEQ ID NO: 106), wherein X3, X5, X9, X13, and Z1 are as defined in Emb. 2, and wherein the side chains of C(*) and PEN(*) are linked by a disulphide bond].

Emb. 3. A compound according to formula (III)

(III)
(SEQ ID NO: 107)
Y2-Z2-X0-X1-X2-X3-X4-X5-X6-X7-X8-X9-X10-X11-X12-X13-Y1;

or salt thereof, preferably a pharmaceutically acceptable salt thereof,
wherein
X0 is DTyr,
X1 is I,
X2 is C,
X3 is Tle or V,
X4 is TrpMe,
X5 is selected from the group consisting of Q, DprAc, and bhAsn,
X6 is D,
X7 is W,
X8 is Sar,
X9 is A or E,
X10 is H,
X11 is R,
X12 is PEN,
X13 is selected from the group consisting of NMeI, DArg, DLys, DThr, DPhe, DTrp, P, DLeu, NMeF, and APCA,
wherein the compound has a disulphide bridge between the sulphur atoms of X2 and X12,
and wherein
   Y1 is selected from the group consisting of OH, K—OH, and NH2, Z2 is a sequence of m amino acid residues independently selected from the group consisting of K, E, and gGlu, m is an integer selected from 1 to 4, and Y2 is hydrogen, with the proviso that when X13 is DTrp, Z2 is not a sequence of m K residues (SEQ ID NO: 112); [i.e. Hy-Z2-DTyr-I-C(*)-X3-TrpMe-X5-D-W-Sar-X9-H-R-PEN(*)-X13-Y1 (SEQ ID NO: 107), wherein Z2, X3, X5, X9, X13, and Y1 are as defined in Emb. 3, and wherein the side chains of C(*) and PEN(*) are linked by a disulphide bond].

Emb. 4. The compound according to embodiment 1, wherein n is equal to three and m is equal to three.

Emb. 5. The compound according to embodiment 1, wherein Z1 is a sequence of three identical amino acid residues (i.e. n=3), wherein the amino acid residue is selected from the group consisting of K, E, and gGlu, and wherein Z2 is a sequence of three identical amino acid residues (i.e. m=3), wherein the amino acid residue is selected from the group consisting of K, E, and gGlu.

Emb. 6. The compound according to embodiment 2, wherein n is equal to three.

Emb. 7. The compound according to embodiment 2, wherein Z1 is a sequence of three identical amino acid residues (i.e. n is 3) wherein the amino acid residue is selected from the group consisting of K, E, and gGlu.

Emb. 7.1. The compound according to embodiment 2, wherein n is an integer equal to 2 (two) or 3 (three) and Z1 is a sequence of n identical amino acid residues wherein the amino acid residue is selected from the group consisting of K, E, and gGlu.

Emb. 8. The compound according to embodiment 3, wherein m is equal to three.

Emb. 9. The compound according to embodiment 3, wherein Z2 is a sequence of three identical amino acid residues (i.e. m is 3) wherein the amino acid residue is selected from the group consisting of K, E, and gGlu.

Emb. 10. The compound according to any one of the preceding embodiments, wherein X3 is Tle.

Emb. 11. The compound according to any one of the preceding embodiments, wherein X13 is selected from the group consisting of NMeI, DArg, DLys, and DThr; preferably DLys or DThr (for clarity, the proviso according to Emb. 1 to 3 does not apply to Emb. 11 or any analogue Emb. wherein X13 may not be DTrp).

Emb. 12. The compound according to embodiment 2, wherein X13 is selected from the group consisting of NMeI, DArg, DLys, and DThr; preferably DLys or DThr.

Emb. 13. The compound according to any one of the preceding embodiments, wherein X3 is Tle and X13 is selected from the group consisting of NMeI, DArg, DLys, and DThr.

Emb. 14. The compound according to embodiment 2, wherein X3 is Tle and X13 is selected from the group consisting of NMeI, DArg, DLys, and DThr, preferably DLys, and DThr.

Emb. 15. The compound according to embodiment 7 or 7.1, wherein X3 is Tle and X13 is selected from the group consisting of NMeI, DArg, DLys, and DThr, preferably DLys, and DThr.

Emb. 16. The compound according to embodiment 8 or 9, wherein X3 is Tle and X13 is selected from the group consisting of NMeI, DArg, DLys, and DThr.

Emb. 17. The compound according to any one of the preceding embodiments, wherein X5 is Q.

Emb. 18. The compound according to any one of the preceding embodiments, wherein X5 is bhAsn.

Emb. 19. The compound according to any one of the preceding embodiments, wherein X5 is DprAc.

Emb. 20. The compound according to embodiment 2, wherein X5 is Q or bhAsn.

Emb. 21. The compound according to embodiment 2, wherein X5 is Q.

Emb. 22. The compound according to embodiment 2, wherein X5 is bhAsn.

Emb. 23. The compound according to embodiment 2, wherein X5 is DprAc.

Emb. 24. The compound according to embodiment 14, wherein X5 is Q.

Emb. 25. The compound according to embodiment 14, wherein X5 is bhAsn.

Emb. 26. The compound according to embodiment 14, wherein X5 is DprAc.

Emb. 27. The compound according to embodiment 15, wherein X5 is Q.

Emb. 28. The compound according to embodiment 15, wherein X5 is bhAsn.

Emb. 29. The compound according to embodiment 15, wherein X5 is DprAc.

Emb. 30. The compound according to embodiment 2, wherein X5 is Q or bhAsn, and X9 is A.

Emb. 31. The compound according to embodiment 14, wherein X5 is Q or bhAsn, and X9 is A.

Emb. 32. The compound according to embodiment 15, wherein X5 is Q or bhAsn, and X9 is A.

Emb. 33. The compound according to embodiment 2, wherein X5 is Q and X9 is A.

Emb. 34. The compound according to embodiment 14, wherein X5 is Q and X9 is A.

Emb. 35. The compound according to embodiment 15, wherein X5 is Q and X9 is A.

Emb. 36. The compound according to embodiment 2, wherein X5 is Q and X9 is E.

Emb. 37. The compound according to embodiment 14, wherein X5 is Q and X9 is E.

Emb. 38. The compound according to embodiment 15, wherein X5 is Q and X9 is E.

Emb. 39. The compound according to embodiment 2, wherein X5 is bhAsn and X9 is A.

Emb. 40. The compound according to embodiment 14, wherein X5 is bhAsn and X9 is A.

Emb. 41. The compound according to embodiment 15, wherein X5 is bhAsn and X9 is A.

Emb. 42. The compound according to embodiment 2, wherein X5 is DprAc and X9 is E.

Emb. 43. The compound according to embodiment 14, wherein X5 is DprAc and X9 is E.

Emb. 44. The compound according to embodiment 15, wherein X5 is DprAc and X9 is E.

Emb. 45. The compound according to any one of the preceding embodiments, wherein Z1 is a sequence of three K (i.e. K-K-K).

Emb. 46. The compound according to any one of the preceding embodiments, wherein Z1 is a sequence of three E (i.e. E-E-E).

Emb. 47. The compound according to any one of the preceding embodiments, wherein Z1 is a sequence of three gGlu (i.e. gGlu-gGlu-gGlu).

Emb. 48. The compound according to embodiment 28, wherein Z1 is a sequence selected in the group consisting of two K (i.e. K-K), three K (i.e. K-K-K), two gGlu (i.e. gGlu-gGlu), and three gGlu (i.e. gGlu-gGlu-gGlu).

Emb. 49. The compound according to embodiment 2, wherein X5 is bhAsn, X13 is DLys or DThr, and Z1 is a sequence of n identical amino acid residues selected from a group consisting of K and gGlu, n is an integer equal to 2 (two) or 3 (three); preferably n is an integer equal to 3 (three).

Emb. 50. The compound according to embodiment 2, wherein X5 is bhAsn, X9 is A, X13 is DLys or DThr, and Z1 is a sequence of n identical amino acid residues selected from a group consisting of K and gGlu, n is an integer equal to 2 (two) or 3 (three); preferably n is an integer equal to 3 (three).

Emb. 51. The compound according to embodiment 2, wherein X5 is DprAc, X13 is DLys or DThr, and Z1 is a sequence of 3 (three) identical amino acid residues selected from a group consisting of K and gGlu.

Emb. 52. The compound according to embodiment 2, wherein X5 is DprAc, X9 is E, X13 is DLys or DThr, and Z1 is a sequence of 3 (three) identical amino acid residues selected from a group consisting of K and gGlu.

### Embodiments A

Emb. A. A compound according to formula (Ia)

(Ia)
(SEQ ID NO: 117)
Y2-Z2-X0-X1-X2-X3-X4-X5-X6-X7-X8-X9-X10-X11-X12-X13-Z1-Y1

or salt thereof, preferably a pharmaceutically acceptable salt thereof,
wherein
X0 is DTyr,
X1 is I,
X2 is C,
X3 is Tle,
X4 is TrpMe,
X5 is bhAsn or DprAc,
X6 is D,
X7 is W,
X8 is Sar,
X9 is A or E,
X10 is H,
X11 is R,
X12 is PEN,
X13 is selected from the group consisting of DArg, DLys, DThr, DPhe, P, and NMeF,
wherein the compound has a disulphide bridge between the sulphur atoms of X2 and X12,
and wherein
   (a) Z1 is a sequence of n amino acid residues independently selected from a group consisting of K, E, gGlu, DLys, and DgGlu, n is an integer selected from 1 to 4, Y1 is OH, Z2 is absent, and Y2 is hydrogen; or
   (b) Z1 is absent, Y1 is selected from the group consisting of OH and K—OH, Z2 is a sequence of m amino acid residues independently selected from a group consisting of K, E, and gGlu, m is an integer selected from 1 to 4, and Y2 is hydrogen (SEQ ID NO: 118).

Emb. A.1. The compound according to embodiment A, wherein X5 is bhAsn.

Emb. A.2. The compound according to embodiment A, wherein X5 is DprAc.

Emb. A.3. The compound according to embodiment A.1 or A.2, wherein X13 is selected from the group consisting of DArg, DLys, and DThr; preferably DLys, and DThr.

Emb. A.4. The compound according to any one of embodiments A.1 to A.3, wherein
(a) Z1 is a sequence of n identical amino acid residues selected from a group consisting of K, E, gGlu, DLys, and DgGlu, n is an integer equal to 2 (two) or 3 (three), Y1 is OH, Z2 is absent, and Y2 is hydrogen; or
(b) Z1 is absent, Y1 is selected from the group consisting of OH and K—OH, Z2 is a sequence of m identical amino acid residues selected from a group consisting of K, E, and gGlu, m is an integer equal to 3 (three), and Y2 is hydrogen.

Emb. A.5. The compound according to any one of embodiments A.1 to A.3, wherein
(a) Z1 is a sequence of n identical amino acid residues selected from a group consisting of K, E, gGlu, DLys, and DgGlu, n is an integer equal to 3 (three), Y1 is OH, Z2 is absent, and Y2 is hydrogen; or
(b) Z1 is absent, Y1 is selected from the group consisting of OH and K—OH, Z2 is a sequence of m identical amino acid residues selected from a group consisting of K, E, and gGlu, m is an integer equal to 3 (three), and Y2 is hydrogen.

Emb. A.6. The compound according to embodiment A, wherein X5 is bhAsn, X13 is DLys or DThr, and (a) Z1 is a sequence of n identical amino acid residues selected from a group consisting of K, gGlu, DLys, and DgGlu, n is an integer equal to 2 (two) or 3 (three), Y1 is OH, Z2 is absent, and Y2 is hydrogen; preferably n is an integer equal to 3 (three).

Emb. A.7. The compound according to embodiment A, wherein X5 is bhAsn, X9 is A, X13 is DLys or DThr, and (a) Z1 is a sequence of n identical amino acid residues selected from a group consisting of K, gGlu, DLys, and DgGlu, n is an integer equal to 2 (two) or 3 (three), Y1 is OH, Z2 is absent, and Y2 is hydrogen; preferably n is an integer equal to 3 (three).

Emb. A.8. The compound according to embodiment A, wherein X5 is DprAc, X13 is DLys or DThr, and (a) Z1 is a sequence of n identical amino acid residues selected from a group consisting of K, gGlu, and DLys, n is an integer equal to 3 (three), Y1 is OH, Z2 is absent, and Y2 is hydrogen.

Emb. A.9. The compound according to embodiment A, wherein X5 is DprAc, X9 is E, X13 is DLys or DThr, and (a) Z1 is a sequence of n identical amino acid residues selected from a group consisting of K, and DLys, n is an integer equal to 3 (three), Y1 is OH, Z2 is absent, and Y2 is hydrogen.

### Embodiments B

Emb. B. A compound according to formula (IV)

(IV)
(SEQ ID NO: 119)
Hy-DTyr-I-C(*)-Tle-TrpMe-X5-D-W-Sar-X9-H-R-PEN(*)-X13-Z1-OH;

or salt thereof, preferably a pharmaceutically acceptable salt thereof, wherein
X5 is selected from the group consisting of Q, DprAc, and bhAsn, preferably DprAc or bhAsn,
X9 is A or E,
X13 is DLys or DThr,
Z1 is a sequence of n amino acid residues independently selected from the group consisting of DLys and DgGlu, and n
is an integer equal to 3 (three); and
wherein the compound has a disulphide bridge between the sulphur atoms of C and PEN.

Emb. B.1. The compound according to embodiment, wherein
Z1 is a sequence consisting of DLys-DLys-DLys or DgGlu-DgGlu-DgGlu.
Emb. B.2. The compound according to embodiment B or B.1, wherein X5 is bhAsn or DprAc.
Emb. B.3. The compound according to embodiment B or B.1, wherein X5 is bhAsn.
Emb. B.4. The compound according to embodiment B or B.1, wherein X5 is DprAc.

### Embodiments I to IV

Emb.I. The compound according to any one of the preceding embodiments, wherein the compound binding to C3 protein and inhibiting complement activation.

Emb. II. The compound according to any one of the preceding embodiments or the pharmaceutically acceptable salt thereof, wherein the compound is for use as a medicament, preferably wherein the compound is for use in a method of treating or preventing a disease disclosed in the present application in a patient in need thereof in.

Emb. III. A method of treatment of a disease disclosed in the present application, preferably an eye or ocular disease, most preferably geographic atrophy (GA), said method comprising the administration of an effective amount of a compound according to any one of the preceding embodiments or a pharmaceutical acceptable salt thereof to a patient in need thereof.

Emb. IV. The use of a compound according to any one of the preceding embodiments or a pharmaceutically acceptable salt thereof in the manufacture of a medicament for inhibiting complement activation and/or for the treatment of a disease disclosed in the present application, preferably an eye or ocular disease, most preferably geographic atrophy (GA).

### Biological Assays and Data

The following examples demonstrate certain specific embodiments of the present invention. The following examples were carried out using standard techniques that are well known and routine to those of skill in the art, except where otherwise described in detail.

### Example 1A: Alternative Pathway (AP) Potency

To monitor inhibition of the complement system in human serum, the peptides were tested using an enzyme immunoassay for the qualitative determination of functional alternative complement pathways in human serum using the WIESLAB® Complement system Screen (Svar Life Science AB, Malmö, Sweden), following the supplier's instructions. The amount of C5b-C9 neoantigen generated is proportional to the functional activity of complement pathways. Final serum concentrations were 5%. The functional potency of the compounds according to the invention and reference compounds outside the invention was tested in a concentration response curve and the IC50 for the inhibition was calculated using a non-linear, 4 parameters curve regression. Due to naturally high C3 concentrations in human serum, the assay is not able to determine IC50 values significantly lower that ~170 nM. Data is summarised in table 1 below.

### Example 1B: Classical Pathway (CP) Potency

To monitor inhibition of the complement system in human serum, the peptides were tested using an enzyme immunoassay for the qualitative determination of functional classical complement pathways in human serum using the WIESLAB® Complement system Screen (Svar Life Science AB, Malmö, Sweden), following the supplier's instructions. The amount of C5b-C9 neoantigen generated is proportional to the functional activity of complement pathways. Final serum concentrations were 1%. The functional potency of the compounds according to the invention and reference compounds outside the invention was tested in a concentration response curve and the IC50 for the inhibition was calculated using a non-linear, 4 parameters curve regression. Due to naturally high C3 concentrations in human serum, the assay is not able to determine IC50 values significantly lower that ~40 nM. Data is summarised in table 1 below.

### Example 1C: Surface Plasmon Resonance Measurements (SPR)

SPR data was collected using a Bruker SPR32 machine (termed “SPR32”). Complement C3c protein (C3c from human Plasma; purchased from Athens research and further purified by size exclusion chromatography in 20 mM TRIS pH 7.5; 150 mM NaCl, 2 mM EDTA) was immobilized by amine coupling on a High Capacity Amine Chip (SPR Affinity Sensor Chips, BRUKER) with a flow rate of 5 μl/min using the following procedure: 1) Activation of active and reference flow cells with EDC/NHS for 420 s; 2) Dilution of C3c to around 0.02 mg/ml using 10 mM sodium acetate pH 5 prior to 150 s or 240 s immobilization; 3) Injection of ethanolamine for 420 s over all flow cells. The running buffer for immobilization was 10 mM HEPES pH 7.4, 150 mM NaCl, 3 mM EDTA, 0.005% Tween-20. All experiments were performed at 25° C. in running buffer with a final DMSO concentration of 2% (v/v). Peptides were diluted from 1 mM DMSO stocks in running buffer and injected at a flow rate of 30 μl/min for 200 s association time with increasing concentrations (9-11 concentrations in Multi Cycle Kinetics or Titration Cycle Kinetics).

SPR data was additionally collected using a Cytiva Biacore T200 machine (termed “Biacore”), using identical C3c immobilization protocols with a High Capacity Amine Chip (Series S Sensor Chip CM5, Cytiva) and running buffer as described above. Peptides were diluted in running buffer from 1 mM DMSO stocks and tested at a flow rate of 30 μl/min in a multi or single cycle kinetics (MCK and SCK) experiment at seven or five different concentrations with 240 s contact time and dissociation time of 1200 s (MCK) or 6000 s (SCK).

The dissociation time was adjusted according to the off rate of each compound. For data analysis, the sensorgram from the reference surface and blank injections was subtracted from the raw data, using the SPR32 Analyzer 4 software or Biacore T200 evaluation software 3.2, respectively. In the case of fast rate constants, the steady-state-affinity model was used to calculate the KD. For slower rate constants, a 1:1 interaction model (Kinetic Titration model), with a term for mass-transport included, was fitted globally to the sensorgram recorded at different compound concentrations. Data is summarised in table 1 below.

While the AP/CP potency assays cannot differentiate compounds with IC50's below ~170/40 nM, respectively, KD affinity determinations by SPR have the advantage of increased dynamic range, allowing identification and ranking of compounds with KD's in the picomolar (pM) range.

The potency and affinity of compounds of the invention and reference compounds outside the invention according to example 1A, 1B and 1C are summarized in table 1 below.

TABLE 1

| | | Potency AP | Potency CP | Affinity SPR |
|---|---|---|---|---|
| Compound No | SEQ ID NO | (nM) | (nM) | KD (nM) |
| 1 | 1 | 264 | 111 | 0.10* |
| 2 | 2 | 534 | 205 | 0.13* |
| 3 | 3 | 592 | 206 | 0.87* |
| 4 | 4 | 498 | 132 | 0.079 |
| 5 | 5 | 518 | 235 | 0.15 |
| 6 | 6 | 492 | 213 | 1.05* |
| 7 | 7 | 211 | 303 | 0.074 |
| 8 | 8 | 268 | 160 | 0.62 |
| 9 | 9 | 215 | 135 | 0.86* |
| 10 | 10 | 269 | 126 | 0.057 |
| 11 | 11 | 265 | 155 | 0.44 |
| 12 | 12 | 274 | 101 | 0.28* |
| 13 | 13 | 337 | 109 | 0.14 |
| 14 | 14 | 346 | 183 | 0.80 |
| 15 | 15 | 286 | 63 | 0.27 |
| 16 | 16 | 259 | 73 | 0.38 |
| 17 | 17 | 289 | 60 | 0.33 |
| 18 | 18 | 450 | 159 | 0.20 |
| 19 | 19 | 266 | 102 | 0.12 |
| 20 | 20 | 277 | 164 | 0.064 |
| 21 | 21 | 340 | 197 | 1.02 |
| 22 | 22 | 294 | 156 | 1.03 |
| 23 | 23 | 278 | 91 | 0.13 |
| 24 | 24 | 238 | 52 | 0.38 |
| 25 | 25 | 258 | 76 | 0.76 |
| 26 | 26 | 265 | 186 | 0.30 |
| 27 | 27 | 288 | 172 | 0.40 |
| 28 | 28 | 252 | 57 | 0.15 |
| 29 | 29 | 269 | 136 | 0.17 |
| 30 | 30 | 268 | 138 | 0.98 |
| 31 | 31 | 266 | 137 | 0.64 |
| 32 | 32 | 286 | 146 | 1.45 |
| 33 | 33 | 324 | 217 | 0.76 |
| 34 | 34 | 331 | 276 | 0.84 |
| 35 | 35 | 177 | 136 | 1.54 |
| 36 | 36 | 328 | 135 | 0.05* |
| 37 | 37 | 336 | 113 | 1.14* |
| 38 | 38 | 272 | 116 | 0.15 |
| 39 | 39 | 270 | 108 | 0.83 |
| 40 | 40 | 365 | 115 | 0.21 |
| 41 | 41 | 265 | 84 | 0.091 |
| 42 | 42 | 353 | 154 | 0.26 |
| 43 | 43 | 264 | 138 | 0.13 |
| 44 | 44 | 197 | 112 | 0.412 |
| 45 | 45 | 245 | 63 | 0.263 |
| 46 | 46 | 306 | 101 | 0.396 |
| 47 | 47 | 258 | 130 | 0.54 |
| 48 | 48 | 271 | 77 | 0.053 |
| 49 | 49 | 307 | 139 | 0.402 |
| 50 | 50 | 269 | 167 | 0.239 |
| 51 | 51 | 314 | 192 | 4.70* |
| 52 | 52 | 538 | 241 | 0.212 |
| 53 | 53 | 214 | 192 | 24.20* |
| 54 | 54 | 133 | 44 | 0.18 |
| 55 | 55 | 270 | 91 | 0.054 |
| 56 | 56 | 266 | 82 | 0.049 |
| 57 | 57 | 252 | 103 | 0.095 |
| 58 | 58 | 384 | 92 | 0.049 |
| 59 | 59 | 537 | 159 | 0.089 |
| 60 | 60 | 413 | 153 | 0.108 |
| 61 | 61 | 404 | 91 | 0.104 |
| 62 | 62 | 301 | 98 | 0.041 |
| 63 | 63 | 259 | 180 | 0.288 |
| 64 | 64 | 266 | 119 | 0.343 |
| 66 | 66 | 291 | 135 | 0.23 |
| 67 | 67 | 542 | 242 | 0.355 |
| 68 | 68 | 266 | 108 | 0.154 |
| 69 | 69 | 260 | 102 | 0.193 |
| 70 | 70 | 248 | 124 | 0.279 |
| 71 | 71 | 322 | 172 | 0.220 |
| 72 | 72 | 388 | 57 | 0.029 |
| 73 | 73 | 283 | 57 | 0.024 |
| 74 | 74 | 238 | 45 | 0.139 |
| 75 | 75 | 267 | 181 | 0.026 |
| 76 | 76 | 271 | 59 | 0.060 |
| 77 | 77 | 279 | 126 | 0.058 |
| 78 | 78 | 298 | 45 | 0.095 |
| 79 | 79 | 458 | 88 | 0.032 |
| 80 | 80 | 267 | 85 | 0.062 |
| 81 | 81 | 330 | 273 | 0.915 |
| 82 | 82 | 278 | 350 | 1.090 |
| 83 | 83 | 409 | 282 | 0.670 |
| 84 | 84 | 443 | 455 | 0.433 |
| 85 | 85 | 219 | 262 | 1.11 |
| 86 | 86 | 590 | 328 | 0.539 |
| 87 | 87 | 263 | 158 | 2.028 |
| 88 | 88 | 258 | 236 | 0.96 |
| 89 | 89 | 386 | 130 | 0.07 |
| 90 | 90 | 256 | 174 | 0.250 |
| 91 | 91 | 292 | 109 | 0.04 |
| 92 | 92 | 403 | 155 | 0.167 |
| 93 | 93 | 256 | 109 | 0.074 |
| 94 | 94 | 260 | 75 | 0.15 |
| 95 | 95 | 257 | 101 | 0.17 |
| 96 | 96 | 266 | 114 | 0.03 |
| 97 | 97 | 262 | 146 | 0.26 |
| 98 | 98 | 346 | 109 | 0.26* |
| 99 | 99 | 313 | 130 | 0.35* |
| 100 | 100 | 256 | 66 | 0.01* |
| 101 | 113 | 340 | 99 | 0.03* |
| 102 | 114 | 311 | 105 | 0.33* |
| 103 | 115 | 318 | 157 | 0.12* |
| Ref. 1 | 101 | 329 | 136 | 0.264 |
| Ref. 2 | 102 | 283 | 98 | 0.108 |
| Ref. 3 | 116 | 186 | 374 | 16.30 |

N/D means not determined.
Affinity SPR data in Table 1 was acquired using the SPR32 system other than where (*) is present; (*) indicates that data was acquired using the Biacore system.

### Example 2. Solubility

The thermodynamic solubility of compounds was measured up to a top concentration of 5 mg/mL by weighing approximately 1 mg of the compound into a syringeless filter vial (polyvinylidene fluoride filter membrane, 0.45 μm, Thompson Instrument Company, CA, part no. 25541) before 200 μL of 100 mM sodium phosphate (Na2HPO4/NaH2PO4) buffer pH 7.0 was added. The suspension was shaken on an orbital shaker at 300 rpm at 25° C. for 2 h. After this period, the pH of the solution or suspension was checked to ensure a target pH of pH 7.0±0.2 of the sample. Subsequently, a separation of the liquid and the solid phase of the incubation mixture was carried out by pushing down the plunger of the filter vial. The filtration step was followed by an immediate dilution of 100 μL of the filtrate in 400 μL of 100 mM sodium phosphate buffer pH 7.0. The concentration of the compound in this solution was determined by UPLC-UV analysis employing a Waters Acquity UPLC system (Waters Corporation, MA) equipped with a FTN sample manager and PDA detector operating at 220 nm wavelength. A freshly prepared solution in 100 mM sodium phosphate buffer pH 7.0 of the respective compound was used as external standard for quantification. The chromatographic analysis was done by injecting 3 μL of sample or standard onto an Acquity UPLC BEH C18 column (1.7 μm, 50×2.1 mm, Waters Corporation, MA, part no. 186002350) operated at 50° C. and eluting with a generic gradient composed of water+0.1 trifluoroacetic acid (Eluent A) and acetonitrile+0.1% trifluoroacetic acid (Eluent B).

| | Time | Flow rate | | |
|---|---|---|---|---|
| | (min) | (mL/min) | % A | % B |
| 1 | Initial | 0.80 | 95.0 | 5.0 |
| 2 | 2.00 | 0.80 | 5.0 | 95.0 |
| 3 | 2.60 | 0.80 | 5.0 | 95.0 |
| 4 | 2.80 | 0.80 | 95.0 | 5.0 |
| 5 | 3.40 | 0.80 | 95.0 | 5.0 |

The quantification of compound in the sample solutions was performed by relating the measured peak area value of the sample to the peak area value of the respective external standard of known concentration. Solubility of compounds according to the invention and reference compounds outside the invention was measured according to example 2 and the results and summarized in table 2 below.

TABLE 2

| Compound No | Solubility (mg/ml) |
|---|---|
| Ref. 1 | 1.60 |
| Ref. 2 | 4.95 |
| Ref. 3 | 0.73 |
| 1 | 1.90 |
| 2 | N/D |
| 3 | 2.00 |
| 4 | 4.80 |
| 5 | 4.20 |
| 6 | 3.40 |
| 7 | 4.70 |
| 8 | 3.70 |
| 9 | 4.30 |
| 10 | 4.20 |
| 11 | 4.10 |
| 12 | 3.60 |
| 13 | 4.40 |
| 14 | 4.20 |
| 15 | 4.10 |
| 16 | 4.70 |
| 17 | 4.80 |
| 18 | ≥5.00 |
| 19 | 4.90 |
| 20 | ≥5.00 |
| 21 | 4.50 |
| 22 | 4.80 |
| 23 | 4.90 |
| 24 | 4.60 |
| 25 | 4.20 |
| 26 | 4.70 |
| 27 | 3.80 |
| 28 | 4.60 |
| 29 | 3.10 |
| 30 | 4.90 |
| 31 | ≥5.00 |
| 32 | ≥5.00 |
| 33 | ≥5.00 |
| 34 | >5.00 |
| 35 | ≥5.00 |
| 36 | ≥5.00 |
| 37 | 4.50 |
| 38 | 4.20 |
| 39 | ≥5.00 |
| 40 | 4.60 |
| 41 | ≥5.00 |
| 42 | 4.80 |
| 43 | ≥5.00 |
| 44 | 4.80 |
| 45 | 4.80 |
| 46 | ≥5.00 |
| 47 | ≥5.00 |
| 48 | ≥5.00 |
| 49 | ≥5.00 |
| 50 | 4.00 |
| 51 | 4.90 |
| 52 | N/D |
| 53 | N/D |
| 54 | 4.70 |
| 55 | 2.40 |
| 56 | ≥5.00 |
| 57 | 4.90 |
| 58 | 4.80 |
| 59 | 4.50 |
| 60 | ≥5.00 |
| 61 | 4.10 |
| 62 | 3.70 |
| 63 | 4.80 |
| 64 | N/D |
| 66 | 4.90 |
| 67 | Precipitation |
| 68 | 4.80 |
| 69 | 4.90 |
| 70 | 4.70 |
| 71 | 3.50 |
| 72 | 4.40 |
| 73 | 4.80 |
| 74 | 4.60 |
| 75 | 4.80 |
| 76 | 4.90 |
| 77 | 4.60 |
| 78 | ≥5.00 |
| 79 | N/D |
| 80 | 4.70 |
| 81 | N/D |
| 82 | 4.60 |
| 83 | 5.00 |
| 84 | 4.70 |
| 85 | 0.90 |
| 86 | 2.80 |
| 87 | 1.70 |
| 88 | 3.80 |
| 89 | 5.00 |
| 90 | 4.70 |
| 91 | 4.70 |
| 92 | N/D |
| 93 | 1.30 |
| 94 | ≥5.00 |
| 95 | 4.60 |
| 96 | 4.90 |
| 97 | 4.80 |
| 98 | 4.30 |
| 99 | 4.60 |
| 100 | ≥5.00 |
| 101 | 4.90 |

N/D: not determined.
“Precipitation” indicates that an accurate solubility determination is not possible due to visible aggregation/precipitation occurring during the experiment.

### Example 3. Chemical Stability

To test the chemical degradation of compounds in solution, samples were prepared by weighing 1 mg of solid material into a 4 mL glass HPLC vial before 50 mM sodium phosphate (Na₂HPO₄/NaH₂PO₄) buffer pH 7.4 was added to dissolve the compound at a target concentration of 1 mg/mL. If required, up to 20% (V/V) acetonitrile were added to facilitate complete dissolution of the solid material. The solution was checked for optical clearness and the pH was measured to ensure a target pH of 7.4±0.2 in the incubation samples. Glass vials containing buffer without compound were used as blank controls. After sealing the vials with a lid, samples were placed into a temperature-controlled chamber at 40° C. and incubated for 14 days in the dark. After that time, vials were taken from the incubation chamber and the content was visually checked for precipitation. If still clearly dissolved, vials were placed into a chilled autosampler at 4-8° C. of a Waters Acquity Premier UPLC system (Waters Corporation, MA) equipped with a FTN sample manager and PDA detector operating at 280 nm wavelength. Control standards of compounds were prepared by freshly dissolving 1 mg of respective solid material in buffer at the day of analysis. All samples and controls were sequentially injected into the UPLC system at 3 μL volume and chromatographically separated on a Kinetex 2.6 μm C8 column (2.6 μm, 150×4.6 mm; Phenomenex, CA, part no. 00F-4497-E0) operated at 50° C. and employing a gradient mobile phase composed of water+0.3% trifluoroacetic acid (Eluent A) and acetonitrile+0.3% trifluoroacetic acid (Eluent B) at a flow rate of 0.5 mL/min.

| | Time | Flow rate | | |
|---|---|---|---|---|
| | (min) | (mL/min) | % A | % B |
| 1 | Initial | 0.50 | 75.0 | 25.0 |
| 2 | 25.00 | 0.50 | 50.0 | 50.0 |
| 3 | 26.00 | 0.50 | 30.0 | 70.0 |
| 4 | 26.50 | 0.50 | 30.0 | 70.0 |
| 5 | 27.00 | 0.50 | 75.0 | 25.0 |

Following measurements, the degree of chemical degradation was calculated by subtracting the relative peak purity of a sample from the respective control standard after blank correction and based on peak area values using EMPOWER 3 (Waters) software.

Chemical stability of some compounds according to the invention and reference compounds outside the invention was measured according to example 3 and the results summarized in table 3 below.

TABLE 3

| Compound No | % degradation |
|---|---|
| Ref. 1 | 2.100** |
| Ref. 2 | 5.700** |
| Ref. 3 | 3.40 |
| 1 | N/D |
| 2 | N/D |
| 3 | N/D |
| 4 | 0.1* |
| 5 | no degradation observed |
| 6 | 0.200 |
| 7 | 1.100 |
| 8 | N/D |
| 9 | no degradation observed** |
| 10 | 0.700** |
| 11 | 0.10 |
| 12 | no degradation observed** |
| 13 | N/D |
| 14 | N/D |
| 15 | 0.80 |
| 16 | 0.20 |
| 17 | 0.70 |
| 18 | no degradation observed |
| 19 | 1.10 |
| 20 | 1.600 |
| 21 | N/D |
| 22 | N/D |
| 23 | 2.600 |
| 24 | N/D |
| 25 | 2.20 |
| 26 | N/D |
| 27 | N/D |
| 28 | 1.70 |
| 29 | N/D |
| 30 | N/D |
| 31 | 2.00 |
| 32 | N/D |
| 33 | N/D |
| 34 | N/D |
| 35 | N/D |
| 36 | 2.000 |
| 37 | no degradation observed |
| 38 | no degradation observed |
| 39 | no degradation observed |
| 40 | 0.100 |
| 41 | 0.1 |
| 42 | no degradation observed |
| 43 | 1.700 |
| 44 | no degradation observed |
| 45 | 0.1 |
| 46 | 0.200 |
| 47 | 1.00 |
| 48 | 0.900 |
| 49 | 1.100 |
| 50 | 2.000 |
| 51 | no degradation observed** |
| 52 | 3.00 |
| 53 | 2.600** |
| 54 | 0.20 |
| 55 | precipitation |
| 56 | 4.60 |
| 57 | 1.10 |
| 58 | precipitation |
| 59 | 0.50 |
| 60 | precipitation |
| 61 | 0.60 |
| 62 | precipitation |
| 63 | 1.400 |
| 64 | 2.300 |
| 66 | 1.70 |
| 67 | precipitation |
| 68 | 1.700 |
| 69 | 1.700 |
| 70 | 1.30 |
| 71 | 3.50 |
| 72 | 2.600 |
| 73 | 1.500 |
| 74 | 1.600 |
| 75 | 1.600 |
| 76 | 0.200 |
| 77 | precipitation |
| 78 | 1.600 |
| 79 | no degradation observed |
| 80 | 0.600 |
| 81 | N/D |
| 82 | 1.900 |
| 83 | N/D |
| 84 | 1.9 |
| 85 | N/D |
| 86 | N/D |
| 87 | 2.700 |
| 88 | precipitation |
| 89 | 0.80 |
| 90 | 0.9 |
| 91 | 1.70 |
| 92 | N/D |
| 93 | N/D |
| 94 | 1.10 |
| 95 | 0.50 |
| 96 | 1.60 |
| 97 | 1.10 |
| 98 | no degradation observed |
| 99 | no degradation observed |
| 100 | 0.70 |
| 101 | 0.50 |
| 103 | no degradation observed |

*determined in 50 mM Hepes buffer pH 7.4.
**determined in Dulbeccos buffer (8.1 mM Na2HPO4, 1.5 mM KH2PO4, 137 mM NaCl, 2.7 mM KCl).
% degradation values at zero or slightly below were denoted as “no degradation observed”.
“precipitation” indicates that during the time course of the 14 day stability measurement, visible precipitation occurred that impaired determination of an accurate percentage of degradation. Consequently, values are not reported.

### EXPERIMENTAL SECTION—PEPTIDE SYNTHESIS

### List Abbreviations

ACM: acetamidomethyl
ACN: acetonitrile
Boc: tert butyloxycarbonyl-
DCM: dichloromethane
DIC: diisopropylcarbodiimide
DIAD: diisopropylazodicarboxylate
DIPEA: diisopropylethylamine
DMF: N,N-dimethylformamide
DTT: dithiothreitol
eq. equivalents
Fmoc: 9H-fluoren-9-ylmethoxycarbonyl-
m/z: mass to charge ratio (MS signal); z is an integer
NMP: N-methyl-2-pyrrolidone
oxyma: ethyl cyano(hydroxyimino)acetate
Pbf: 2,2,4,6,7-pentamethyl-2,3-dihydro-1-benzofuran-5-sulfonyl-
Pfp: pentafluorophenyl-
Rt: retention time
RT: room temperature
tBu: tert butyl-
TFA: trifluoroacetic acid
Trt: triphenylmethyl-(trityl-)

All the other abbreviations are according to common general knowledge of a skilled in the art.

### General Procedure for Solid-Phase Peptide Synthesis

Peptides were synthesized using solid-phase peptide synthesis via Fmoc/tBu chemistry as exemplified in Chan, W. C.; White, P. D. “Fmoc Solid-Phase Synthesis: A Practical Approach”, Oxford University Press, Oxford, 2000; and Benoiton, N. L. “Chemistry of Peptide Synthesis”, CRC Press, New York, 2006. During peptide chain elongation, the α-amino group of each amino acid was protected with a 9H-fluoren-9-ylmethoxycarbonyl group (Fmoc). To avoid any side reactions during the chain elongation steps, any reactive amino acid side chains also carry acid-labile protecting groups, effectively masking the reactive groups until removal upon treatment with strong acid. After completion of each coupling step, the Fmoc group of the N-terminal amino acid was removed with piperidine, and the resin was thoroughly washed to prepare for the coupling of the subsequent Fmoc-protected amino acid derivative.

Two types of resins were used for synthesis, Rink Amide AM resin (0.25 mmol, 100-200 mesh, 0.4 mmol/g loading, EMD Biosciences), and H-AA-loaded 2-chlorotrityl resin (0.25 mmol, 100-200 mesh, 0.57 mmol/g loading, Zerenex).

The following protected amino acids were used: Fmoc-Lys(Boc)-OH, Fmoc-Glu(OtBu)-OH Fmoc-gGlu(OtBu)-OH, Fmoc-Tle-OH, Fmoc-Tba-OH, Fmoc-DNle-OH, Fmoc-Sar-OH, Fmoc-APCA(Boc)-OH, Fmoc-NMePhe-OH, Fmoc-NMeLeu-OH, Fmoc-NMeArg-OH, Fmoc-Pro-OH, Fmoc-DPro-OH, Fmoc-NMeTrp-OH, Fmoc-NMeIle-OH, Fmoc-NEtGly-OH, Fmoc-DLeu-OH, Fmoc-DThr(tBu)-OH, Fmoc-DArg(Pbf)-OH, Fmoc-DTrp(Boc)-OH, Fmoc-PEN(Trt)-OH, Fmoc-Trp(Me)-OH, Fmoc-D1NaI—OH, Fmoc-Asn(Trt)-OH, Fmoc-Arg(Pbf)-OH, Fmoc-Glu-OtBu, Fmoc-His(Trt)-OH, Fmoc-Aib-OH, Fmoc-DprAc-OH, Fmoc-bhAsn-OH, Boc/Fmoc-DTyr(tBu)-OH, Fmoc-Cys(Trt)-OH, Fmoc-Gln(Trt)-OH, Fmoc-Cys(ACM)-OH, Fmoc-PEN(ACM)-OH. The L-form of the amino acid building blocks was utilized if not specified otherwise.

N,N′-Diisopropylcarbodiimide (DIC) with ethyl cyano(hydroxyimino)acetate (Oxyma) were used as coupling agents to form the amide bond between the free amino terminus of the resin-bound protected peptide and the carboxylic acid of the Fmoc-protected amino acid.

The amino acid building blocks, Fmoc-Glu-OtBu, oxyma, DIC, and all reagents were purchased from standard suppliers, e.g. Bachem AG, Merck KGaA (Novabiochem®, Sigma-Aldrich®), ABCR GmbH, Iris Biotec GmbH. All compounds were obtained as TFA salts.

### Synthesis Method 1 (S01)

Peptides were synthesized on a Liberty Blue™ synthesizer from CEM Corporation, using standard solid-phase synthesis (SPPS) using Fmoc/tBu chemistry.

Rink Amide AM resin (0.25 mmol scale, 100-200 mesh, 0.4 mmol/g loading, EMD Biosciences) was used. Coupling of amino acids was carried out by addition of DMF solutions of suitably protected amino acid building block (0.2 mol/l, 4 eq.), oxyma (1 mol/l, 4 eq) and DIC (1 mol/l, 1 ml, 8 eq) to the resin and heating of the resulting suspension to 90° C. by microwave irradiation for 4 minutes. For the coupling of Fmoc-Arg(Pbf)-OH, and Fmoc-Pro-OH, the coupling cycle was repeated twice (double coupling, 2×4 min at 90° C.). For the coupling of Fmoc-PEN(Trt)-OH on Fmoc-NMe-Ile-OH was repeated 4 times with a capping step at the end by using acetic anhydride for 5 min at RT. Fmoc-His(Trt)-OH was coupled at 50° C. (2×12 min). Removal of the Fmoc protective group was performed using an excess of 20% piperidine in DMF for 1 min at 90° C.; this step was repeated for the removal of the first Fmoc group. After completion of peptide chain assembly, the resin was carefully washed with DCM and dried prior to cleavage. Concomitant resin cleavage and side chain deprotection was performed using a mixture of TFA/water/triisopropylsilane (95:5:2.5, ca 15 mL) with an addition of 250 mg DTT. The mixture was stirred for 60 min at RT. Crude peptides were precipitated with cold petrolether:diethyl ether (1:1), centrifugated (4600 rpm at 5° C. for 10 min). Crude peptide was dissolved in ACN:H2O (1:1) and dried via lyophilization.

### Synthesis Method 2 (S02)

Peptides were synthesized on a Liberty Blue™ synthesizer from CEM Corporation, using standard solid-phase synthesis using Fmoc/tBu chemistry.

Preloaded 2-chlorotrityl (CTC) resins were used, gGlu(OtBu)- or Lys(Boc)-loaded 2-chlorotrityl resin. The first amino acid was preloaded manually according to the following procedure: To a solution of 2-CTC Resin (0.5 mmol, 1.00 eq, Sub 0.5 mmol/g) and Fmoc-protected amino acid (1.00 eq) in DCM (20 mL) was added DIPEA (4.00 eq), the mixture was agitated with N2 at 25° C. for 2.5 h. Then MeOH (1.00 mL) was added in the resin and agitated with N2 at 25° C. for 0.5 h. Then the mixture was filtered to obtain the resin. The resin was washed with DMF (5×20 mL). Coupling of subsequent amino acids was performed by adding DMF solutions of protected amino acids (0.2 mol/l, 4 eq), oxyma (1 mol/l, 4 eq) with DIPEA (0.1 mol/l, 0.4 eq) and DIC (1 mol/l, 1 ml, 8 eq) to the resin. The resulting suspension was heated to 50° C. by microwave irradiation for 10 minutes. For the coupling of Fmoc-Arg(Pbf)-OH, and Fmoc-Pro-OH, the coupling cycle was repeated (double coupling, 2×4 min at 50° C.). For the coupling of Fmoc-PEN(Trt)-OH on Fmoc-NMeI-OH was repeated 4 times with a capping step at the end by using acetic anhydride for 5 min at room temperature. Fmoc-His(Trt)-OH was coupled at 50° C. (2×12 min). Removal of the Fmoc protective group was performed using an excess of 20% piperidine/DMF for 1 min at 50° C.; this step was repeated for the removal of the first Fmoc group. After completion of peptide chain assembly, the resin was carefully washed with DCM and dried prior to cleavage. Concomitant resin cleavage and side chain deprotection was performed using a mixture of TFA/water/triisopropylsilane (95:5:2.5 with an addition of 250 mg DTT in ca. 15 mL). The mixture was stirred for 60 min at RT. Crude peptides were precipitated with cold petrolether:diethyl ether (1:1), centrifugated (4600 rpm at 5° C. for 10 min). Crude peptide was dissolved in ACN:H2O (1:1) and dried via lyophilization.

### Cyclization Method 1 (C01)

The linear crude cleaved peptide comprising two thiol containing amino acids (selected from Cys, and PEN) was dissolved in 4 mg/mL in H2O:ACN and stirred vigorously at room temperature. A solution of DIAD in DMF (0.5 eq./mL) was slowly added over 2-5 min, and the reaction was left to stir for a further 2-12 hours. The solvent was removed via lyophilization to be afterwards directly purified.

### Cyclization Method 2 (C02)

A solution of crude peptide (50 mg) in H2O (35 mL) and ACN (15 mL) was adjusted to pH 2 with HCl (1M in water). I2 in MeOH (0.5 M) was added dropwise at 20° C. until a yellow color persisted. The mixture was stirred at 20° C. for 2 min, and analytical check by LCMS was performed to confirm the correctly formed product. Solvent was removed via lyophilization to be afterwards directly purified.

### Cyclization Method 3 (C03)

On-resin disulfide bond formation was performed after the assembly of the linear peptide on resin (0.25 mmol). This involved thiol-containing amino acids (selected from Cys, and PEN) with Acm protecting group. The resin-bound peptide was washed with DMF (5×), and a solution of 8 mL of DMF:anisole (9.5:0.5) was added to resin. After swelling for 5-10 min, Thallium[III]trifluoroacetate (1.2 eq) was added. The reaction was stirred for 2-12 hours, followed by washing with DMF 20 mL (3×), DCM 20 mL (3×). Concomitant resin cleavage and side chain deprotection was performed using a mixture of TFA/water/triisopropylsilane (95:5:2.5) for 3 hours at RT. Crude peptides were precipitated with cold petrolether:diethyl ether (1:1), centrifuged (4600 rpm at 5° C. for 10 min). Crude peptide was dissolved in ACN:H2O (1:1) and dried via lyophilization.

### Purification Method 1 (P01)

Crude peptides were dissolved in DMF/acetonitrile/water and purified by reversed phase chromatography using an Agilent preparative HPLC-MS System with preparative pumps G7161B, G7111B, G7110B, a diode array detector G7115A, a mass-spectrometer G6135B and an autosampler/fraction collector G7159B. The stationary phase was a Phenomenex LUNA C8 10 μm Prep Column (50×250 mm). The peptides were eluted with a linear gradient of eluent A (H2O supplemented with 0.1% TFA) and eluent B (ACN supplemented with 0.1% TFA) at a flow rate of 120 ml/min and a temperature of 40° C. Fractions containing the desired product at sufficient purity were pooled and lyophilized. The final product was characterized by HPLC-MS (see below analytical methods A01, A02).

### Purification Method 2 (P02)

Crude peptides were dissolved in DMF/acetonitrile/water and purified by reversed phase chromatography using GILSON preparative HPLC System with preparative pumps AP-MOD (max. flow rate: 200 ml/min), a diode array detector ECOM Flash 10 and a fraction collector GILSON GX 281. The stationary phase was a Phenomenex LUNA C8 10 μm Prep Column (50×250 mm). The peptides were eluted with a focused gradient using water (eluent A) and acetonitrile (eluent B) at a flow rate of 120 ml/min at a temperature of 40° C.; modifier solution was added in ‘at-column dilution’ mode to maintain a constant concentration of 0.1% TFA within the mobile phase. Fractions containing the desired product at sufficient purity were pooled and lyophilized. The final product was characterized by HPLC-MS (see below analytical methods A01, A02).

### Analytical Method (A01)

The purity of peptide products and mass identity was assessed by analytical HPLC-MS on a Kinetex C8 column (4.6 mm×150 mm, 2.6 um, Phenomenex) using Agilent 1290 HPLC system equipped with Time-of-Flight (TOF) MS G6230 (A02). Analysis was performed by gradient elution with eluent A (in H2O supplemented with 0.3% TFA) and eluent B (ACN supplemented with 0.26% TFA) at a temperature of 40° C. Details of the gradient and flow rates are summarized in table A01. Chromatographic retention times and most prominent m/z signals (+ESI mode) were recorded, where m=molecular mass and z=net charge of the observed molecular species. Peptide purities (relative peak areas at 214 nm) were in the range from 80% to 99%, preferably greater than 95%.

### Analytical Method (A02)

The purity of peptide products and mass identity was assessed by analytical HPLC-MS on a Kinetex C8 column (4.6 mm×150 mm, 2.6 um, Phenomenex) using Agilent 1290 HPLC system equipped with Time-of-Flight (TOF) MS G6230 (A02). Analysis was performed by gradient elution with eluent A (H2O supplemented with 0.3% TFA) and eluent B (ACN supplemented with 0.26% TFA) at a temperature of 40° C. Details of the gradient and flow rates are summarized in table A02. Chromatographic retention times and most prominent m/z signals (+ESI mode) were recorded, where m=molecular mass and z=net charge of the observed molecular species. Peptide purities (relative peak areas at 214 nm) were in the range from 80% to 99%, preferably greater than 95%.

TABLE A01

| | |
|---|---|
| Method Name: | A01 |
| Device: | Agilent 1290 with TOF-MS G6230B |
| Column: | Phenomenex Kinetex C8, 4.6 × 150 mm; 2.6 μm |

Gradient:

| Time | % Eluent A | % Eluent B | Flow | Temp |
|---|---|---|---|---|
| [min] | [Water 0.3% TFA (v/v)] | [ACN 0.26% TFA (v/v)] | [ml/min] | [° C.] |
| 0.0 | 85.0 | 15.0 | 0.5 | 40.0 |
| 50.0 | 35.0 | 65.0 | 0.5 | 40.0 |
| 52.0 | 10.0 | 90.0 | 1.0 | 40.0 |
| 53.0 | 85.0 | 15.0 | 1.0 | 40.0 |
| 55.0 | 85.0 | 15.0 | 1.0 | 40.0 |

TABLE A02

| | |
|---|---|
| Method Name: | A02 |
| Device: | Agilent 1290 with TOF-MS G6230B |
| Column: | Phenomenex Kinetex C8, 4.6 × 150 mm; 2.6 μm |

Gradient:

| Time | % Eluent A | % Eluent B | Flow | Temp |
|---|---|---|---|---|
| [min] | [Water 0.3% TFA (v/v)] | [ACN 0.26% TFA (v/v)] | [ml/min] | [° C.] |
| 0.0 | 80.0 | 20.0 | 0.5 | 40.0 |
| 30.0 | 50.0 | 50.0 | 0.5 | 40.0 |
| 32.0 | 10.0 | 90.0 | 1.0 | 40.0 |
| 33.0 | 80.0 | 20.0 | 1.0 | 40.0 |
| 35.0 | 80.0 | 20.0 | 1.0 | 40.0 |

### Reference Compound: Ref. 1 (No Part of the Invention)

MW (calculated): 1787.839 Da

Amino acid sequence:
(SEQ ID NO: 101)
DTyr-I-C(*)-V-TrpMe-Q-D-W-Sar-A-H-R-C(*)-NMeI-NH2

Synthesis and cyclization methods: S01, C02 or C03
Analytical method: A01
Rt: 23.976 min
Observed mass: 894.928 Da, [M+2H]/2

### Reference Compound: Ref. 2 (No Part of the Invention)

MW (calculated): 2172.124 Da

Amino acid sequence:
(SEQ ID NO: 102)
DTyr-I-C(*)-V-TrpMe-Q-D-W-Sar-A-H-R-C(*)-NMeI-K-K-K-NH2

Synthesis and cyclization methods: S01, C02 or C03
Analytical method: A02
Rt: 19.864 min
Observed mass: 1087.067 Da, [M+2H]/2

### Reference Compound: Ref. 3. (No Part of the Invention)

MW (calculated): 1654.749 Da

Amino acid sequence:
(SEQ ID NO: 116)
Ac-I-C(*)-V-TrpMe-Q-D-W-G-A-H-R-PEN(*)-T-NH2

Synthesis and cyclization methods: S01, C01
Analytical method: A01
Rt: 23.583 min
Observed mass: 828.381 Da, [M+2H]/2

### Compound: 1

MW (calculated): 1987.966 Da

Amino acid sequence:
(SEQ ID NO: 1)
DTyr-I-C(*)-Tle-TrpMe-Q-D-W-Sar-A-H-R-PEN(*)-
DArg-K-OH

Synthesis and cyclization methods: S02, C01
Analytical method: A01
Rt: 21.938 min
Observed mass: 994.991 Da, [M+2H]/2

### Compound: 2

MW (calculated): 1958.965 Da

Amino acid sequence:
(SEQ ID NO: 2)
DTyr-I-C(*)-Tle-TrpMe-Q-D-W-Sar-A-H-R-PEN(*)-
NMeI-K-OH

Synthesis and cyclization methods: S02, C01
Analytical method: A01
Rt: 21.608 min
Observed mass: 980.489 Da, [M+2H]/2

### Compound: 3

MW (calculated): 1987.966 Da

Amino acid sequence:
(SEQ ID NO: 3)
DTyr-I-C(*)-Tle-TrpMe-DprAc-D-W-Sar-A-H-R-PEN(*)-DArg-K-OH

Synthesis and cyclization methods: S02, C01
Analytical method: A01
Rt: 23.031 min
Observed mass: 994.991 Da, [M+2H]/2

### Compound: 4

MW (calculated): 2215.155 Da

Amino acid sequence:
(SEQ ID NO: 4)
DTyr-I-C(*)-Tle-TrpMe-Q-D-W-Sar-A-H-R-PEN(*)-NMeI-K-K-K-OH

Synthesis and cyclization methods: S02, C02
Analytical method: A02
Rt: 14.245 min
Observed mass: 739.39 Da, [M+3H]/3

### Compound: 5

MW (calculated): 2217.998 Da

Amino acid sequence:
(SEQ ID NO: 5)
DTyr-I-C(*)-Tle-TrpMe-Q-D-W-Sar-A-H-R-PEN(*)-NMeI-E-E-E-OH

Synthesis and cyclization methods: S02, C02
Analytical method: A02
Rt: 16.995 min
Observed mass: 1110.01 Da, [M+2H]/2

### Compound: 6

MW calculated: 2217.998 Da

Amino acid sequence:
(SEQ ID NO: 6)
DTyr-I-C(*)-Tle-TrpMe-Q-D-W-Sar-A-H-R-PEN(*)-NMeI-gGlu-gGlu-gGlu-OH

Synthesis and cyclization methods: S02, C02
Analytical method: A02
Rt: 17.055 min
Observed mass: 1110 Da, [M+2H]/2

### Compound: 7

MW (calculated): 2189.103 Da

Amino acid sequence:
(SEQ ID NO: 7)
DTyr-I-C(*)-Tle-TrpMe-Q-D-W-Sar-A-H-R-PEN(*)-DThr-K-K-K-OH

Synthesis and cyclization methods: S02, C01 or C02
Analytical method: A01
Rt: 21.46 min
Observed mass: 1095.55 Da, [M+2H]/2

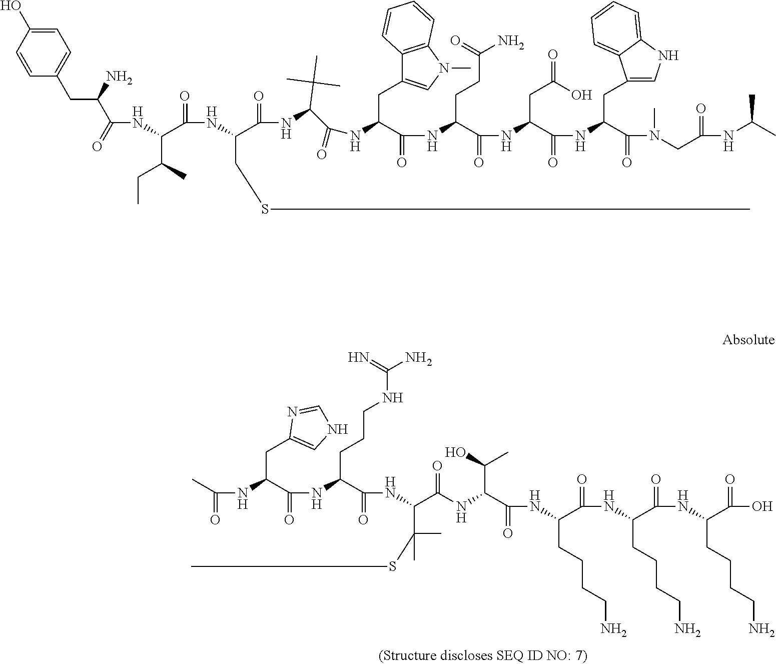

### Compound: 8

MW (calculated): 2191.946 Da

Amino acid sequence:
(SEQ ID NO: 8)
DTyr-I-C(*)-Tle-TrpMe-Q-D-W-Sar-A-H-R-PEN(*)-DThr-E-E-E-OH

Synthesis and cyclization methods: S02, C02
Analytical method: A02
Rt: 18.133 min
Observed mass: 1096.98 Da, [M+2H]/2

### Compound: 9

MW (calculated): 2191.946 Da

Amino acid sequence:
(SEQ ID NO: 9)
DTyr-I-C(*)-Tle-TrpMe-Q-D-W-Sar-A-H-R-PEN(*)-DThr-gGlu-gGlu-gGlu-OH

Synthesis and cyclization methods: S02, C02
Analytical method: A02
Rt: 17.935 min
Observed mass: 1096.98 Da, [M+2H]/2

### Compound: 10

MW (calculated): 2244.156 Da

Amino acid sequence:
(SEQ ID NO: 10)
DTyr-I-C(*)-Tle-TrpMe-Q-D-W-Sar-A-H-R-PEN(*)-DArg-K-K-K-OH

Synthesis and cyclization methods: S02, C02
Analytical method: A02
Rt: 15.948 min
Observed mass: 749.06 Da, [M+3H]/3

### Compound: 11

MW (calculated): 2246.999 Da

Amino acid sequence:
(SEQ ID NO: 11)
DTyr-I-C(*)-Tle-TrpMe-Q-D-W-Sar-A-H-R-PEN(*)-DArg-E-E-E-OH

Synthesis and cyclization methods: S02, C02
Analytical method: A02
Rt: 16.694 min
Observed mass: 1124.51 Da, [M+2H]/2

### Compound: 12

MW (calculated): 2246.999 Da

Amino acid sequence:
(SEQ ID NO: 12)
DTyr-I-C(*)-Tle-TrpMe-Q-D-W-Sar-A-H-R-PEN(*)-DArg-gGlu-gGlu-gGlu-OH

Synthesis and cyclization methods: S02, C02
Analytical method: A02
Rt: 16.697 min
Observed mass: 1124.51 Da, [M+2H]/2

### Compound: 13

MW (calculated): 2201.139 Da

Amino acid sequence:
(SEQ ID NO: 13)
DTyr-I-C(*)-Tle-TrpMe-Q-D-W-Sar-A-H-R-PEN(*)-DLeu-K-K-K-OH

Synthesis and cyclization methods: S02, C02
Analytical method: A02
Rt: 17.891 min
Observed mass: 1101.57 Da, [M+2H]/2

### Compound: 14

MW (calculated): 2203.982 Da

Amino acid sequence:
(SEQ ID NO: 14)
DTyr-I-C(*)-Tle-TrpMe-Q-D-W-Sar-A-H-R-PEN(*)-DLeu-E-E-E-OH

Synthesis and cyclization methods: S02, C02
Analytical method: A02
Rt: 21.031 min
Observed mass: 1103 Da, [M+2H]/2

### Compound: 15

MW (calculated): 2203.982 Da

Amino acid sequence:
(SEQ ID NO: 15)
DTyr-I-C(*)-Tle-TrpMe-Q-D-W-Sar-A-H-R-PEN(*)-DLeu-gGlu-gGlu-gGlu-OH

Synthesis and cyclization methods: S02, C02
Analytical method: A02
Rt: 20.649 min
Observed mass: 1103 Da, [M+2H]/2

### Compound: 16

MW (calculated): 2276.977 Da

Amino acid sequence:
(SEQ ID NO: 16)
DTyr-I-C(*)-Tle-TrpMe-Q-D-W-Sar-A-H-R-PEN(*)-DTrp-E-E-E-OH

Synthesis and cyclization methods: S02, C02
Analytical method: A02
Rt: 20.96 min
Observed mass: 1139.5 Da, [M+2H]/2

### Compound: 17

MW (calculated): 2276.977 Da

Amino acid sequence:
(SEQ ID NO: 17)
DTyr-I-C(*)-Tle-TrpMe-Q-D-W-Sar-A-H-R-PEN(*)-DTrp-gGlu-gGlu-gGlu-OH

Synthesis and cyclization methods: S02, C02
Analytical method: A02
Rt: 20.441 min
Observed mass: 1139.5 Da, [M+2H]/2

### Compound: 18

MW (calculated): 2346.093 Da

Amino acid sequence:
(SEQ ID NO: 18)
DTyr-I-C(*)-Tle-TrpMe-Q-D-W-Sar-A-H-R-PEN(*)-NMeI-K-gGlu-gGlu-gGlu-OH

Synthesis and cyclization methods: S02, C02
Analytical method: A02
Rt: 15.101 min
Observed mass: 1174.05 Da, [M+2H]/2

### Compound: 19

MW (calculated): 2405.072 Da

Amino acid sequence:
(SEQ ID NO: 19)
DTyr-I-C(*)-Tle-TrpMe-Q-D-W-Sar-A-H-R-PEN(*)-DTrp-K-gGlu-gGlu-gGlu-OH

Synthesis and cyclization methods: S02, C02
Analytical method: A02
Rt: 19.172 min
Observed mass: 1203.54 Da, [M+2H]/2

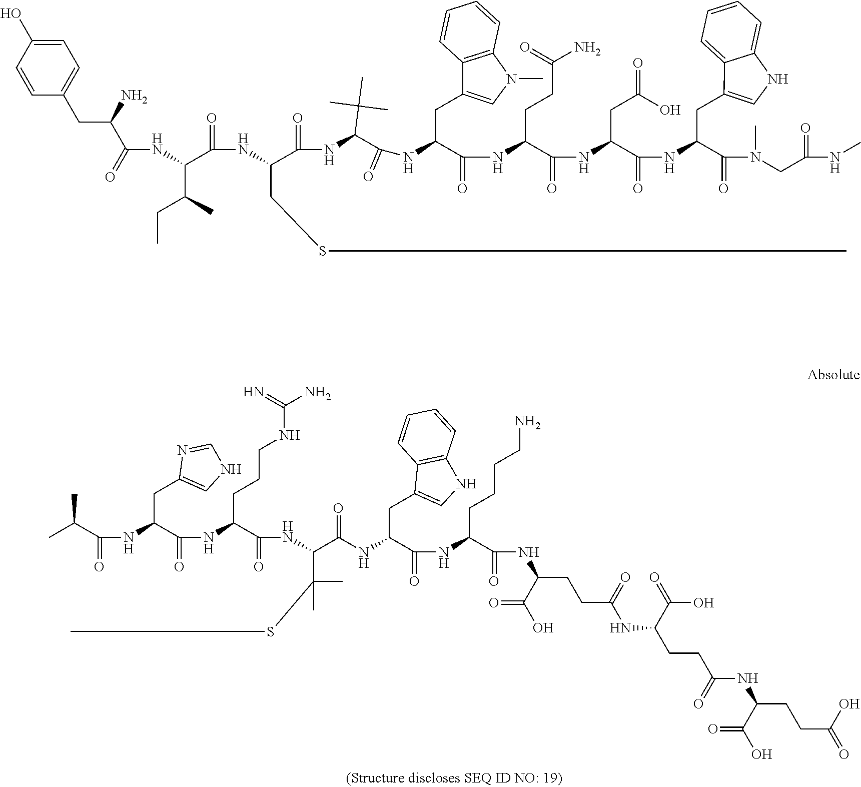

### Compound: 20

MW (calculated): 2214.171 Da
Amino acid sequence: K-K-K-DTyr-I-C(*)-Tle-TrpMe-Q-D-W-Sar-A-H-R-PEN(*)-NMeI-NH2 (SEQ ID NO: 20)
Synthesis and cyclization methods: 501, C02
Analytical method: A02
Rt: 15.465 min
Observed mass: 1108.09 Da, [M+2H]/2

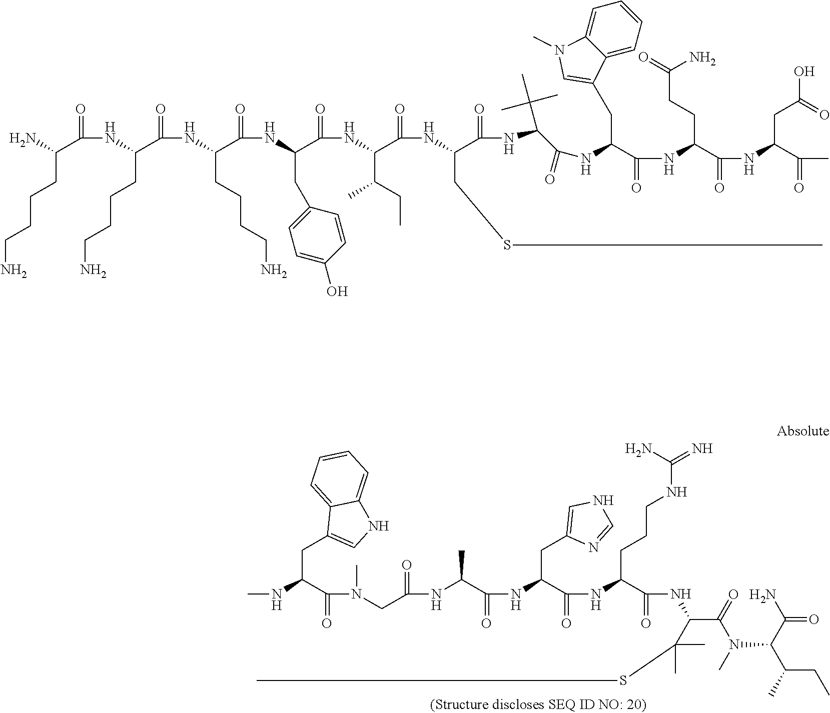

### Compound: 21

MW (calculated): 2217.014 Da

Amino acid sequence:
(SEQ ID NO: 21)
E-E-E-DTyr-I-C(*)-Tle-TrpMe-Q-D-W-Sar-A-H-R-PEN(*)-NMeI-NH2

Synthesis and cyclization methods: 501, C02
Analytical method: A02
Rt: 18.399 min
Observed mass: 1109.51 Da, [M+2H]/2

### Compound: 22

MW (calculated): 2217.014 Da

Amino acid sequence:
(SEQ ID NO: 22)
gGlu-gGlu-gGlu-DTyr-I-C(*)-Tle-TrpMe-Q-D-W-Sar-A-H-R-PEN(*)-NMeI-NH2

Synthesis and cyclization methods: 501, C02
Analytical method: A02
Rt: 18.409 min
Observed mass: 1109.51 Da, [M+2H]/2

### Compound: 23

MW (calculated): 2188.119 Da

Amino acid sequence:
(SEQ ID NO: 23)
K-K-K-DTyr-I-C(*)-Tle-TrpMe-Q-D-W-Sar-A-H-R-PEN(*)-DThr-NH2

Synthesis and cyclization methods: 501, C02
Analytical method: A02
Rt: 14.804 min
Observed mass: 1095.07 Da, [M+2H]/2

### Compound: 24

MW (calculated): 2190.962 Da

Amino acid sequence:
(SEQ ID NO: 24)
E-E-E-DTyr-I-C(*)-Tle-TrpMe-Q-D-W-Sar-A-H-R-PEN(*)-DThr-NH2

Synthesis and cyclization methods: 501, C02
Analytical method: A02
Rt: 17.719 min
Observed mass: 1096.49 Da, [M+2H]/2

### Compound: 25

MW (calculated): 2190.962 Da

Amino acid sequence:
(SEQ ID NO: 25)
gGlu-gGlu-gGlu-DTyr-I-C(*)-Tle-TrpMe-Q-D-W-Sar-A-H-R-PEN(*)-DThr-NH2

Synthesis and cyclization methods: 501, C02
Analytical method: A02
Rt: 17.723 min
Observed mass: 1096.49 Da, [M+2H]/2

### Compound: 26

MW (calculated): 2243.172 Da

Amino acid sequence:
(SEQ ID NO: 26)
K-K-K-DTyr-I-C(*)-Tle-TrpMe-Q-D-W-Sar-A-H-R-PEN(*)-DArg-NH2

Synthesis and cyclization methods: 501, C02
Analytical method: A02
Rt: 13.559 min
Observed mass: 748.73 Da, [M+3H]/3

### Compound: 27

MW (calculated): 2246.015 Da

Amino acid sequence:
(SEQ ID NO: 27)
E-E-E-DTyr-I-C(*)-Tle-TrpMe-Q-D-W-Sar-A-H-R-PEN(*)-DArg-NH2

Synthesis and cyclization methods: 501, C02
Analytical method: A02
Rt: 16.271 min
Observed mass: 1124.02 Da, [M+2H]/2

### Compound: 28

MW (calculated): 2246.015 Da

Amino acid sequence:
(SEQ ID NO: 28)
gGlu-gGlu-gGlu-DTyr-I-C(*)-Tle-TrpMe-Q-D-W-Sar-A-H-R-PEN(*)-DArg-NH2

Synthesis and cyclization methods: 501, C02
Analytical method: A02
Rt: 16.541 min
Observed mass: 1124.01 Da, [M+2H]/2

### Compound: 29

MW (calculated): 2200.155 Da

Amino acid sequence:
(SEQ ID NO: 29)
K-K-K-DTyr-I-C(*)-Tle-TrpMe-Q-D-W-Sar-A-H-R-PEN(*)-DLeu-NH2

Synthesis and cyclization methods: S01, C02
Analytical method: A02
Rt: 17.554 min
Observed mass: 1101.09 Da, [M+2H]/2

### Compound: 30

MW (calculated): 2202.998 Da

Amino acid sequence:
(SEQ ID NO: 30)
E-E-E-DTyr-I-C(*)-Tle-TrpMe-Q-D-W-Sar-A-H-R-PEN(*)-DLeu-NH2

Synthesis and cyclization methods: 501, C02
Analytical method: A02
Rt: 20.869 min
Observed mass: 1102.51 Da, [M+2H]/2

### Compound: 31

MW (calculated): 2202.998 Da

Amino acid sequence:
(SEQ ID NO: 31)
gGlu-gGlu-gGlu-DTyr-I-C(*)-Tle-TrpMe-Q-D-W-Sar-A-H-R-PEN(*)-DLeu-NH2

Synthesis and cyclization methods: S01, C02
Analytical method: A02
Rt: 20.866 min
Observed mass: 1102.51 Da, [M+2H]/2

### Compound: 32

MW (calculated): 2275.993 Da

Amino acid sequence:
(SEQ ID NO: 32)
E-E-E-DTyr-I-C(*)-Tle-TrpMe-Q-D-W-Sar-A-H-R-PEN(*)-DTrp-NH2

Synthesis and cyclization methods: S01, C02
Analytical method: A02
Rt: 20.536 min
Observed mass: 1139.01 Da, [M+2H]/2

### Compound: 33

MW calculated: 2275.993 Da

Amino acid sequence:
(SEQ ID NO: 33)
pgGlu-gGlu-gGlu-DTyr-I-C(*)-Tle-TrpMe-Q-D-W-Sar-A-H-R-PEN(*)-DTr-NH2

Synthesis and cyclization methods: S01, C02
Analytical method: A02
10 Rt: 20.449 min
Observed mass: 1139 Da, [M+2H]/2

### Compound: 34

MW (calculated): 2346.093 Da

Amino acid sequence:
(SEQ ID NO: 34)
gGlu-gGlu-gGlu-DTyr-I-C(*)-Tle-TrpMe-Q-D-W-Sar-A-H-R-PEN(*)-NMeI-K-OH

Synthesis and cyclization methods: S02, C02
Analytical method: A02
Rt: 16.274 min
Observed mass: 1174.05 Da, [M+2H]/2

### Compound: 35

MW (calculated): 2405.072 Da

Amino acid sequence:
(SEQ ID NO: 35)
gGlu-gGlu-gGlu-DTyr-I-C(*)-Tle-TrpMe-Q-D-W-Sar-A-H-R-PEN(*)-DTrp-K-OH

Synthesis and cyclization methods: S02, C02
Analytical method: A02
Rt: 18.88 min
Observed mass: 1203.54 Da, [M+2H]/2

### Compound: 36

MW (calculated): 2201.139 Da

Amino acid sequence:
(SEQ ID NO: 36)
DTyr-I-C(*)-V-TrpMe-Q-D-W-Sar-A-H-R-PEN(*)-NMeI-K-K-K-OH

Synthesis and cyclization methods: S02, C02
Analytical method: A02
Rt: 13.587 min
Observed mass: 734.72 Da, [M+3H]/3

### Compound: 37

MW (calculated): 2203.982 Da

Amino acid sequence:
(SEQ ID NO: 37)
DTyr-I-C(*)-V-TrpMe-Q-D-W-Sar-A-H-R-PEN(*)-NMeI-gGlu-gGlu-gGlu-OH

Synthesis and cyclization methods: S02, C02
Analytical method: A02
Rt: 16.112 min
Observed mass: 1103 Da, [M+2H]/2

### Compound: 38

MW (calculated): 2175.087 Da

Amino acid sequence:
(SEQ ID NO: 38)
DTyr-I-C(*)-V-TrpMe-Q-D-W-Sar-A-H-R-PEN(*)-DThr-K-K-K-OH

Synthesis and cyclization methods: S02, C02
Analytical method: A02
Rt: 15.531 min
Observed mass: 1088.55 Da, [M+2H]/2

### Compound: 39

MW (calculated): 2177.930 Da

Amino acid sequence:
(SEQ ID NO: 39)
DTyr-I-C(*)-V-TrpMe-Q-D-W-Sar-A-H-R-PEN(*)-DThr-gGlu-gGlu-gGlu-OH

Synthesis and cyclization methods: S02, C02
Analytical method: A02
Rt: 16.979 min
Observed mass: 1089.97 Da, [M+2H]/2

### Compound: 40

MW (calculated): 2276.003 Da

Amino acid sequence:
(SEQ ID NO: 40)
DTyr-I-C(*)-Tle-TrpMe-Q-D-W-Sar-E-H-R-PEN(*)-NMeI-gGlu-gGlu-gGlu-OH

Synthesis and cyclization methods: S02, C02
Analytical method: A02
Rt: 17.162 min
Observed mass: 1139.01 Da, [M+2H]/2

### Compound: 41

MW (calculated): 2273.160 Da

Amino acid sequence:
(SEQ ID NO: 41)
DTyr-I-C(*)-Tle-TrpMe-Q-D-W-Sar-E-H-R-PEN(*)-NMeI-K-K-K-OH

Synthesis and cyclization methods: S02, C02
Analytical method: A02
Rt: 14.441 min
Observed mass: 758.73 Da, [M+3H]/3

### Compound: 42

MW (calculated): 2249.951 Da

Amino acid sequence:
(SEQ ID NO: 42)
DTyr-I-C(*)-Tle-TrpMe-Q-D-W-Sar-E-H-R-PEN(*)-DThr-gGlu-gGlu-gGlu-OH

Synthesis and cyclization methods: S02, C02
Analytical method: A02
Rt: 17.833 min
Observed mass: 1125.99 Da, [M+2H]/2

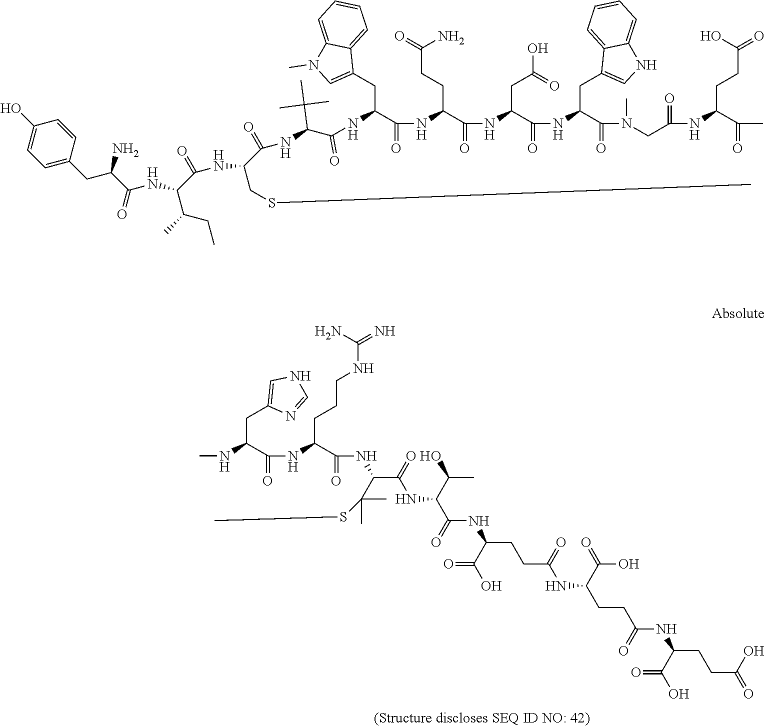

### Compound: 43

MW (calculated): 2247.108 Da

Amino acid sequence:
(SEQ ID NO: 43)
DTyr-I-C(*)-Tle-TrpMe-Q-D-W-Sar-E-H-R-PEN(*)-DThr-K-K-K-OH

Synthesis and cyclization methods: S02, C02
Analytical method: A02
Rt: 16.469 min
Observed mass: 1124.56 Da, [M+2H]/2

### Compound: 44

MW (calculated): 2191.946 Da

Amino acid sequence:
(SEQ ID NO: 44)
DTyr-I-C(*)-Tle-TrpMe-bhAsn-D-W-Sar-A-H-R-PEN(*)-DThr-gGlu-gGlu-gGlu-OH

Synthesis and cyclization methods: S02, C01 or C02
Analytical method: A01
Rt: 22.1 mi
Observed mass: 1096.97 Da, [M+2H]/2

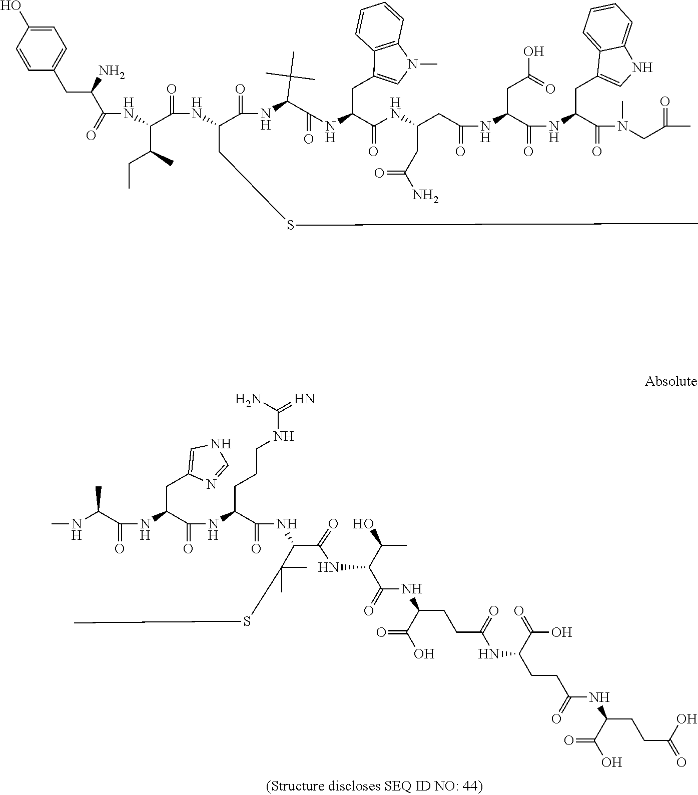

### Compound: 45

MW (calculated): 2251.982 Da

Amino acid sequence:
(SEQ ID NO: 45)
DTyr-I-C(*)-Tle-TrpMe-Q-D-W-Sar-A-H-R-PEN(*)-NMeF-gGlu-gGlu-gGlu-OH

Synthesis and cyclization methods: S02, C02
Analytical method: A02
10 Rt: 19.323 min
Observed mass: 1127 Da, [M+2H]/2

### Compound: 46

MW (calculated): 2187.951 Da

Amino acid sequence:
(SEQ ID NO: 46)
DTyr-I-C(*)-Tle-TrpMe-Q-D-W-Sar-A-H-R-PEN(*)-P-gGlu-gGlu-gGlu-OH

Synthesis and cyclization methods: S02, C02
Analytical method: A02
Rt: 17.111 min
Observed mass: 1094.98 Da, [M+2H]/2

### Compound: 47

MW (calculated): 2218.993 Da

Amino acid sequence:
(SEQ ID NO: 47)
DTyr-I-C(*)-Tle-TrpMe-Q-D-W-Sar-A-H-R-PEN(*)-DLys-gGlu-gGlu-gGlu-OH

Synthesis and cyclization methods: S02, C02
Analytical method: A02
Rt: 16.294 min
Observed mass: 1110.5 Da, [M+2H]/2

### Compound: 48

MW (calculated): 2216.150 Da

Amino acid sequence:
(SEQ ID NO: 48)
DTyr-I-C(*)-Tle-TrpMe-Q-D-W-Sar-A-H-R-PEN(*)-DLys-K-K-K-OH

Synthesis and cyclization methods: S02, C02
Analytical method: A02
Rt: 15.549 min
Observed mass: 739.72 Da, [M+3H]/3

### Compound: 49

MW (calculated): 2237.966 Da

Amino acid sequence:
(SEQ ID NO: 49)
DTyr-I-C(*)-Tle-TrpMe-Q-D-W-Sar-A-H-R-PEN(*)-DPhe-gGlu-gGlu-gGlu-OH

Synthesis and cyclization methods: S02, C02
Analytical method: A02
Rt: 20.92 min
Observed mass: 1119.99 Da, [M+2H]/2

### Compound: 50

MW (calculated): 2244.156 Da

Amino acid sequence:
(SEQ ID NO: 50)
DTyr-I-C(*)-Tle-TrpMe-DprAc-D-W-Sar-A-H-R-PEN(*)-DArg-K-K-K-OH

Synthesis and cyclization methods: S02, C01
Analytical method: A01
Rt: 21.695 min
Observed mass: 1123.08 Da, [M+2H]/2

### Compound: 51

MW (calculated): 2246.999 Da

Amino acid sequence:
(SEQ ID NO: 51)
DTyr-I-C(*)-Tle-TrpMe-DprAc-D-W-Sar-A-H-R-PEN(*)-DArg-gGlu-gGlu-gGlu-OH

Synthesis and cyclization methods: S02, C01
Analytical method: A01
Rt: 23.11 min
Observed mass: 1124.503 Da, [M+2H]/2

### Compound: 52

MW (calculated): 2215.155 Da

Amino acid sequence:
(SEQ ID NO: 52)
DTyr-I-C(*)-Tle-TrpMe-DprAc-D-W-Sar-A-H-R-PEN(*)-NMeI-K-K-K-OH

Synthesis and cyclization methods: S02, C01
Analytical method: A01
Rt: 20.806 min
Observed mass: 1108.58 Da, [M+2H]/2

### Compound: 53

MW (calculated): 2191.946 Da

Amino acid sequence:
(SEQ ID NO: 53)
DTyr-I-C(*)-Tle-TrpMe-DprAc-D-W-Sar-A-H-R-PEN(*)-DThr-gGlu-gGlu-gGlu-OH

Synthesis and cyclization methods: S02, C01
Analytical method: A01
Rt: 24.043 min
Observed mass: 1096.979 Da, [M+2H]/2

### Compound: 54

MW (calculated): 2273.160 Da

Amino acid sequence:
(SEQ ID NO: 54)
DTyr-I-C(*)-Tle-TrpMe-DprAc-D-W-Sar-E-H-R-PEN(*)-NMeI-K-K-K-OH

Synthesis and cyclization methods: S02, C02
Analytical method: A02
Rt: 15.807 min
Observed mass: 758.72 Da, [M+3H]/3

### Compound: 55

MW (calculated): 2235.123 Da

Amino acid sequence:
(SEQ ID NO: 55)
DTyr-I-C(*)-Tle-TrpMe-Q-D-W-Sar-A-H-R-PEN(*)-DPhe-K-K-K-OH

Synthesis and cyclization methods: S02, C01
Analytical method: A01
Rt: 23.34 min
Observed mass: 1118.57 Da, [M+2H]/2

### Compound: 56

MW (calculated): 2185.108 Da

Amino acid sequence:
(SEQ ID NO: 56)
DTyr-I-C(*)-Tle-TrpMe-Q-D-W-Sar-A-H-R-PEN(*)-P-K-K-K-OH

Synthesis and cyclization methods: S02, C01
Analytical method: A01
Rt: 20.152 min
Observed mass: 1093.56 Da, [M+2H]/2

### Compound: 57

MW (calculated): 2249.139 Da

Amino acid sequence:
(SEQ ID NO: 57)
DTyr-I-C(*)-Tle-TrpMe-Q-D-W-Sar-A-H-R-PEN(*)-NMeF-K-K-K-OH

Synthesis and cyclization methods: S02, C02
Analytical method: A02
Rt: 15.968 min
Observed mass: 750.72 Da, [M+3H]/3

### Compound: 58

MW (calculated): 2274.155 Da

Amino acid sequence:
(SEQ ID NO: 58)
DTyr-I-C(*)-Tle-TrpMe-Q-D-W-Sar-E-H-R-PEN(*)-DLys-K-K-K-OH

Synthesis and cyclization methods: S02, C01
Analytical method: A01
Rt: 20.664 min
Observed mass: 1138.08 Da, [M+2H]/2

### Compound: 59

MW (calculated): 2243.113 Da

Amino acid sequence:
(SEQ ID NO: 59)
DTyr-I-C(*)-Tle-TrpMe-Q-D-W-Sar-E-H-R-PEN(*)-P-K-K-K-OH

Synthesis and cyclization methods: S02, C01
Analytical method: A01
Rt: 20.158 min
Observed mass: 1122.55 Da, [M+2H]/2

### Compound: 60

MW (calculated): 2307.145 Da

Amino acid sequence:
(SEQ ID NO: 60)
DTyr-I-C(*)-Tle-TrpMe-Q-D-W-Sar-E-H-R-PEN(*)-NMeF-K-K-K-OH

Synthesis and cyclization methods: S02, C02
Analytical method: A02
Rt: 16.015 min
Observed mass: 770.05 Da, [M+3H]/3

### Compound: 61

MW (calculated): 2302.162 Da

Amino acid sequence:
(SEQ ID NO: 61)
DTyr-I-C(*)-Tle-TrpMe-Q-D-W-Sar-E-H-R-PEN(*)-DArg-K-K-K-OH

Synthesis and cyclization methods: S02, C02
Analytical method: A02
Rt: 15.969 min
Observed mass: 768.39 Da, [M+3H]/3

### Compound: 62

MW (calculated): 2293.129 Da

Amino acid sequence:
(SEQ ID NO: 62)
DTyr-I-C(*)-Tle-TrpMe-Q-D-W-Sar-E-H-R-PEN(*)-DPhe-K-K-K-OH

Synthesis and cyclization methods: S02, C01
Analytical method: A01
Rt: 23.216 min
Observed mass: 1147.57 Da, [M+2H]/2

### Compound: 63

MW (calculated): 2189.103 Da

Amino acid sequence:
(SEQ ID NO: 63)
DTyr-I-C(*)-Tle-TrpMe-DprAc-D-W-Sar-A-H-R-PEN(*)-DThr-K-K-K-OH

Synthesis and cyclization methods: S02, C01
Analytical method: A01
Rt: 22.63 min
Observed mass: 1095.557 Da, [M+2H]/2

### Compound: 64

MW (calculated): 2216.150 Da

Amino acid sequence:
(SEQ ID NO: 64)
DTyr-I-C(*)-Tle-TrpMe-DprAc-D-W-Sar-A-H-R-PEN(*)-DLys-K-K-K-OH

Synthesis and cyclization methods: S02, C01
Analytical method: A01
Rt: 21.527 min
Observed mass: 1109.08 Da, [M+2H]/2

### Compound: 66

MW (calculated): 2249.139 Da

Amino acid sequence:
(SEQ ID NO: 66)
DTyr-I-C(*)-Tle-TrpMe-DprAc-D-W-Sar-A-H-R-PEN(*)-NMeF-K-K-K-OH

Synthesis and cyclization methods: S02, C02
Analytical method: A02
Rt: 16.81 min
Observed mass: 750.72 Da, [M+3H]/3

### Compound: 67

MW (calculated): 2235.123 Da

Amino acid sequence:
(SEQ ID NO: 67)
DTyr-I-C(*)-Tle-TrpMe-DprAc-D-W-Sar-A-H-R-PEN(*)-DPhe-K-K-K-OH

Synthesis and cyclization methods: S02, C01
Analytical method: A01
Rt: 23.176 min
Observed mass: 1118.57 Da, [M+2H]/2

### Compound: 68

MW (calculated): 2247.108 Da

Amino acid sequence:
(SEQ ID NO: 68)
DTyr-I-C(*)-Tle-TrpMe-DprAc-D-W-Sar-E-H-R-PEN(*)-DThr-K-K-K-OH

Synthesis and cyclization methods: S02, C01
Analytical method: A01
Rt: 21.555 min
Observed mass: 1124.56 Da, [M+2H]/2

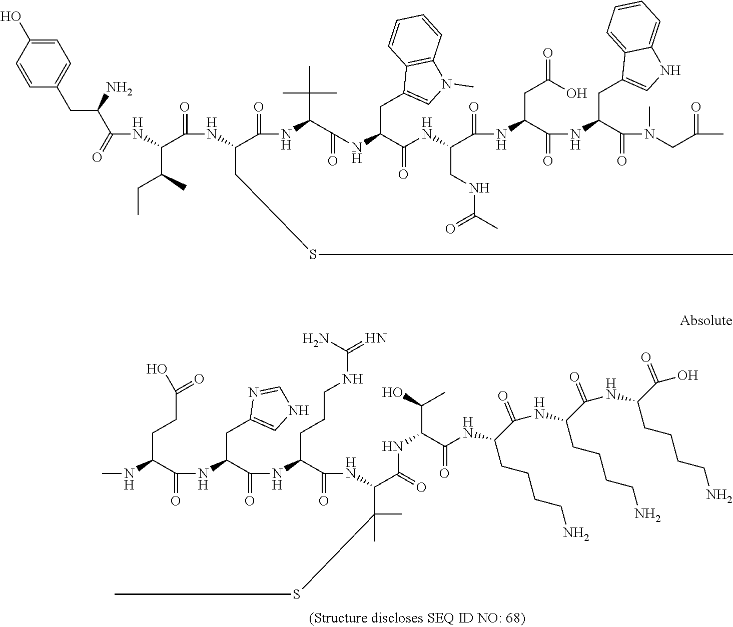

### Compound: 69

MW (calculated): 2274.155 Da

Amino acid sequence:
(SEQ ID NO: 69)
DTyr-I-C(*)-Tle-TrpMe-DprAc-D-W-Sar-E-H-R-PEN(*)-DLys-K-K-K-OH

Synthesis and cyclization methods: S02, C01
Analytical method: A01
Rt: 20.783 min
Observed mass: 1138.08 Da, [M+2H]/2

### Compound: 70

MW (calculated): 2243.113 Da

Amino acid sequence:
(SEQ ID NO: 70)
DTyr-I-C(*)-Tle-TrpMe-DprAc-D-W-Sar-E-H-R-PEN(*)-P-K-K-K-OH

Synthesis and cyclization methods: S02, C01
Analytical method: A01
Rt: 20.49 min
Observed mass: 1122.56 Da, [M+2H]/2

### Compound: 71

MW (calculated): 2293.129 Da

Amino acid sequence:
(SEQ ID NO: 71)
DTyr-I-C(*)-Tle-TrpMe-DprAc-D-W-Sar-E-H-R-PEN(*)-DPhe-K-K-K-OH

Synthesis and cyclization methods: S02, C01
Analytical method: A01
Rt: 23.811 min
Observed mass: 1147.56 Da, [M+2H]/2

### Compound: 72

MW (calculated): 2189.103 Da

Amino acid sequence:
(SEQ ID NO: 72)
DTyr-I-C(*)-Tle-TrpMe-bhAsn-D-W-Sar-A-H-R-PEN(*)-DThr-K-K-K-OH

Synthesis and cyclization methods: S02, C01
Analytical method: A01
Rt: 20.21 min
Observed mass: 1095.56 Da, [M+2H]/2

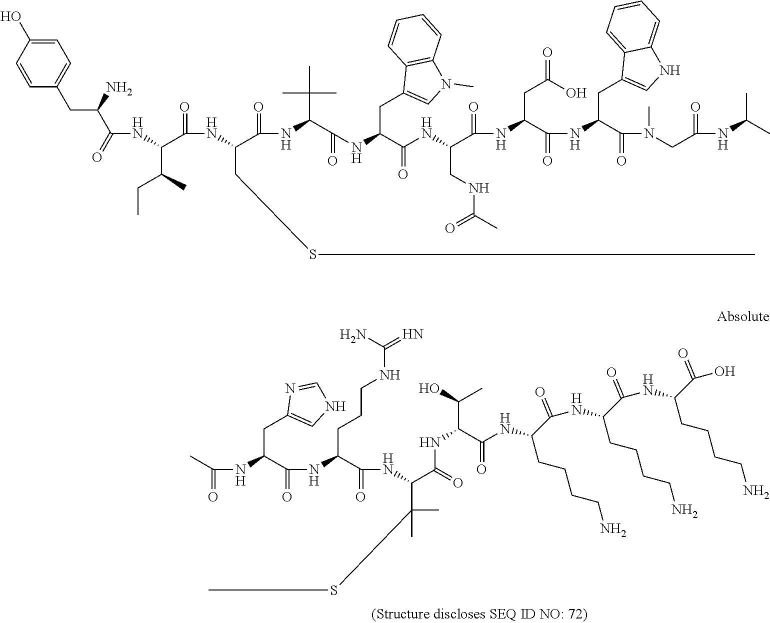

### Compound: 73

MW (calculated): 2216.150 Da

Amino acid sequence:
(SEQ ID NO: 73)
DTyr-I-C(*)-Tle-TrpMe-bhAsn-D-W-Sar-A-H-R-PEN(*)-DLys-K-K-K-OH

Synthesis and cyclization methods: S02, C01
Analytical method: A01
Rt: 18.634 min
Observed mass: 1109.08 Da, [M+2H]/2

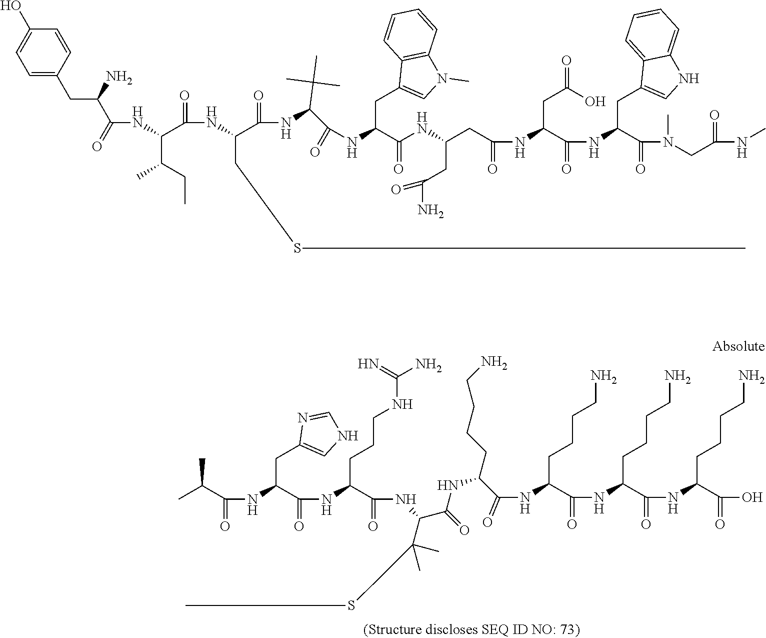

### Compound: 74

MW (calculated): 2185.108 Da

Amino acid sequence:
(SEQ ID NO: 74)
DTyr-I-C(*)-Tle-TrpMe-bhAsn-D-W-Sar-A-H-R-PEN(*)-P-K-K-K-OH

Synthesis and cyclization methods: S02, C01
Analytical method: A01
Rt: 19.22 min
Observed mass: 729.374 Da, [M+2H]/2

### Compound: 75

MW (calculated): 2244.156 Da

Amino acid sequence:
(SEQ ID NO: 75)
DTyr-I-C(*)-Tle-TrpMe-bhAsn-D-W-Sar-A-H-R-PEN(*)-DArg-K-K-K-OH

Synthesis and cyclization methods: S02, C01
Analytical method: A01
Rt: 19.831 min
Observed mass: 749.06 Da, [M+3H]/3

### Compound: 76

MW (calculated): 2235.123 Da

Amino acid sequence:
(SEQ ID NO: 76)
DTyr-I-C(*)-Tle-TrpMe-bhAsn-D-W-Sar-A-H-R-
PEN(*)-DPhe-K-K-K-OH

Synthesis and cyclization methods: S02, C01
Analytical method: A01
Rt: 22.096 min
Observed mass: 1118.56 Da, [M+2H]/2

### Compound: 77

MW (calculated): 2247.108 Da

Amino acid sequence:
(SEQ ID NO: 77)
DTyr-I-C(*)-Tle-TrpMe-bhAsn-D-W-Sar-E-H-R-
PEN(*)-DThr-K-K-K-OH

Synthesis and cyclization methods: S02, C01
Analytical method: A01
Rt: 20.099 min
Observed mass: 1124.56 Da, [M+2H]/2

### Compound: 78

MW (calculated): 2274.155 Da

Amino acid sequence:
(SEQ ID NO: 78)
DTyr-I-C(*)-Tle-TrpMe-bhAsn-D-W-Sar-E-H-R-
PEN(*)-DLys-K-K-K-OH

Synthesis and cyclization methods: S02, C01
Analytical method: A01
Rt: 19.194 min
Observed mass: 759.056 Da, [M+2H]/2

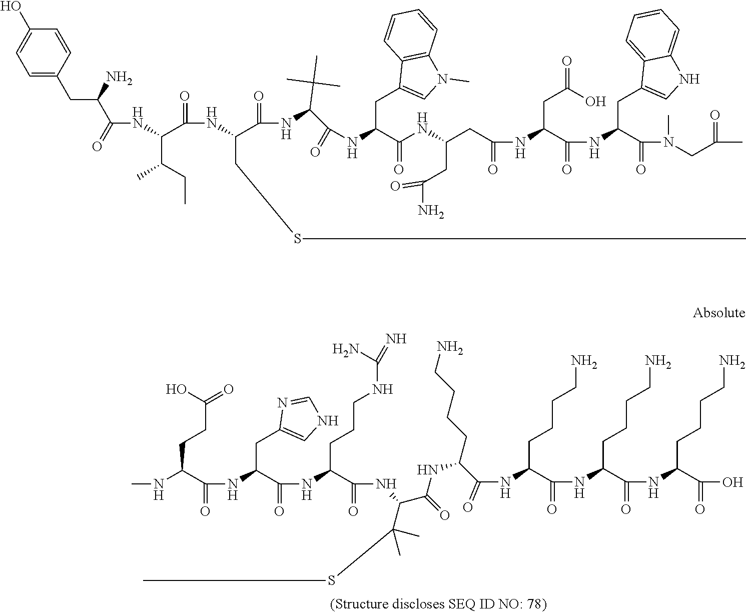

### Compound: 79

MW (calculated): 2272.140 Da

Amino acid sequence:
(SEQ ID NO: 79)
DTyr-I-C(*)-Tle-TrpMe-bhAsn-D-W-Sar-E-H-R-
PEN(*)-APCA-K-K-K-OH

Synthesis and cyclization methods: S02, C01
Analytical method: A01
Rt: 18.954 mi
Observed mass: 758.386 Da, [M+2H]/2

### Compound: 80

MW (calculated): 2293.129 Da

Amino acid sequence:
(SEQ ID NO: 80)
DTyr-I-C(*)-Tle-TrpMe-bhAsn-D-W-Sar-E-H-R-
PEN(*)-DPhe-K-K-K-OH

Synthesis and cyclization methods: S02, C01
Analytical method: A01
Rt: 22.026 min
Observed mass: 1147.57 Da, [M+2H]/2

### Compound: 81

MW (calculated): 2244.156 Da

Amino acid sequence:
(SEQ ID NO: 81)
K-K-K-DTyr-I-C(*)-Tle-TrpMe-DprAc-D-W-Sar-A-H-R-
PEN(*)-DArg-OH

Synthesis and cyclization methods: S02, C01
Analytical method: A01
Rt: 19.926 min
Observed mass: 1123.07 Da, [M+2H]/2

### Compound: 82

MW (calculated): 21893._1293 Da

Amino acid sequence:
(SEQ ID NO: 82)
K-K-K-DTyr-I-C(*)-Tle-TrpMe-DprAc-D-W-Sar-A-H-R-
PEN(*)-DThr-OH

Synthesis and cyclization methods: S02, C01
Analytical method: A01
Rt: 20.884 min
Observed mass: 1095.55 Da, [M+2H]/2

### Compound: 83

MW (calculated): 2216.150 Da

Amino acid sequence:
(SEQ ID NO: 83)
K-K-K-DTyr-I-C(*)-Tle-TrpMe-DprAc-D-W-Sar-A-H-R-
PEN(*)-DLys-OH

Synthesis and cyclization methods: S02, C01
Analytical method: A01
Rt: 19.587 min
Observed mass: 1109.07 Da, [M+2H]/2

### Compound: 84

MW (calculated): 2185.108 Da

Amino acid sequence:
(SEQ ID NO: 84)
K-K-K-DTyr-I-C(*)-Tle-TrpMe-DprAc-D-W-Sar-A-H-R-
PEN(*)-P-OH

Synthesis and cyclization methods: S02, C01 or C02
Analytical method: A01
Rt: 20.98 min
Observed mass: 1093.55 Da, [M+2H]/2

### Compound: 85

MW (calculated): 2235.123 Da

Amino acid sequence:
(SEQ ID NO: 85)
K-K-K-DTyr-I-C(*)-Tle-TrpMe-DprAc-D-W-Sar-A-H-R-
PEN(*)-DPhe-OH

Synthesis and cyclization methods: S02, C02
Analytical method: A02
Rt: 18.456 min
Observed mass: 1118.57 Da, [M+2H]/2

### Compound: 86

MW (calculated): 2302.162 Da

Amino acid sequence:
(SEQ ID NO: 86)
K-K-K-DTyr-I-C(*)-Tle-TrpMe-DprAc-D-W-Sar-E-H-R-
PEN(*)-DArg-OH

Synthesis and cyclization methods: S02, C01
Analytical method: A01
Rt: 19.752 min
Observed mass: 768.39 Da, [M+2H]/2

### Compound: 87

MW (calculated): 2247.108 Da

Amino acid sequence:
(SEQ ID NO: 87)
K-K-K-DTyr-I-C(*)-Tle-TrpMe-DprAc-D-W-Sar-E-H-R-
PEN(*)-DThr-OH

Synthesis and cyclization methods: S02, C01
Analytical method: A01
Rt: 20.724 min
Observed mass: 1124.55 Da, [M+2H]/2

### Compound: 88

MW (calculated): 2274.155 Da

Amino acid sequence:
(SEQ ID NO: 88)
K-K-K-DTyr-I-C(*)-Tle-TrpMe-DprAc-D-W-Sar-E-H-R-
PEN(*)-DLys-OH

Synthesis and cyclization methods: S02, C01
Analytical method: A01
Rt: 19.445 min
Observed mass: 1138.08 Da, [M+2H]/2

### Compound: 89

MW (calculated): 2302.162 Da

Amino acid sequence:
(SEQ ID NO: 89)
K-K-K-DTyr-I-C(*)-Tle-TrpMe-bhAsn-D-W-Sar-E-H-R-
PEN(*)-DArg-OH

Synthesis and cyclization methods: S02, C01
Analytical method: A01
Rt: 17.444 min
Observed mass: 768.39 Da, [M+3H]/3

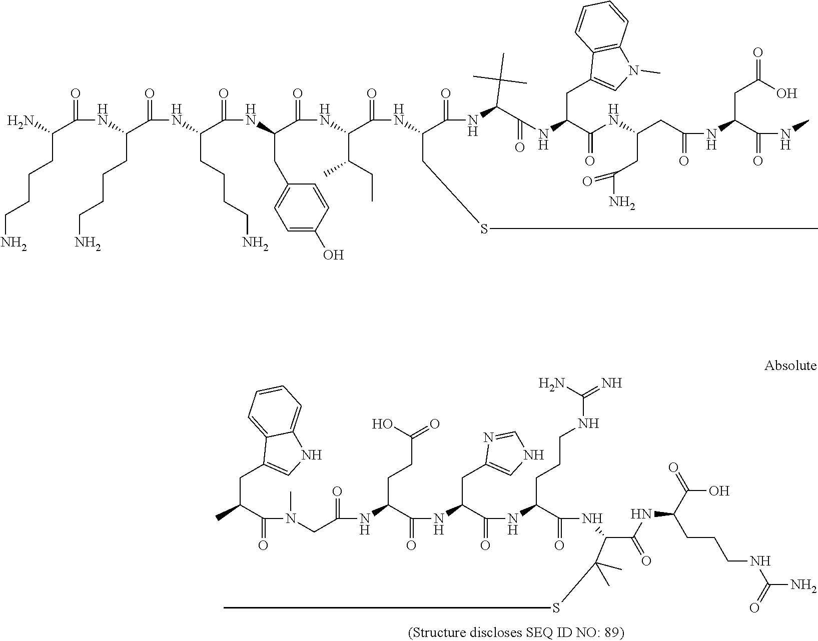

### Compound: 90

MW (calculated): 2247.108 Da

Amino acid sequence:
(SEQ ID NO: 90)
K-K-K-DTyr-I-C(*)-Tle-TrpMe-bhAsn-D-W-Sar-E-H-R-
PEN(*)-DThr-OH

Synthesis and cyclization methods: S02, C01
Analytical method: A01
Rt: 18.723 min
Observed mass: 1124.56 Da, [M+2H]/2

### Compound: 91

MW (calculated): 2274.155 Da

Amino acid sequence:
(SEQ ID NO: 91)
K-K-K-DTyr-I-C(*)-Tle-TrpMe-bhAsn-D-W-Sar-E-H-R
-PEN(*)-DLys-OH

Synthesis and cyclization methods: S02, C01
Analytical method: A01
Rt: 17.082 min
Observed mass: 1138.08 Da, [M+2H]/2

### Compound: 92

MW (calculated): 2243.113 Da

Amino acid sequence:
(SEQ ID NO: 92)
K-K-K-DTyr-I-C(*)-Tle-TrpMe-bhAsn-D-W-Sar-E-H-R
-PEN(*)-P-OH

Synthesis and cyclization methods: S02, C01
Analytical method: A01
Rt: 18.688 min
Observed mass: 1122.56 Da, [M+2H]/2

### Compound: 93

MW (calculated): 2293.129 Da

Amino acid sequence:
(SEQ ID NO: 93)
K-K-K-DTyr-I-C(*)-Tle-TrpMe-bhAsn-D-W-Sar-E-H-R
-PEN(*)-DPhe-OH

Synthesis and cyclization methods: S02, C01
Analytical method: A01
Rt: 21.743 min
Observed mass: 1147.57 Da, [M+2H]/2

### Compound: 94

MW (calculated): 2189.1027 Da

Amino acid sequence:
(SEQ ID NO: 94)
DTyr-I-C*- Tle-TrpMe-Q-D-W-Sar-A-H-R
-PEN(*)-DThr-DLys-DLys-DLys-OH

Synthesis and cyclization methods: S02, C02
Analytical method: A02
Rt: 16.65 min
Observed mass: 1095.554 Da, [M+2H]/2

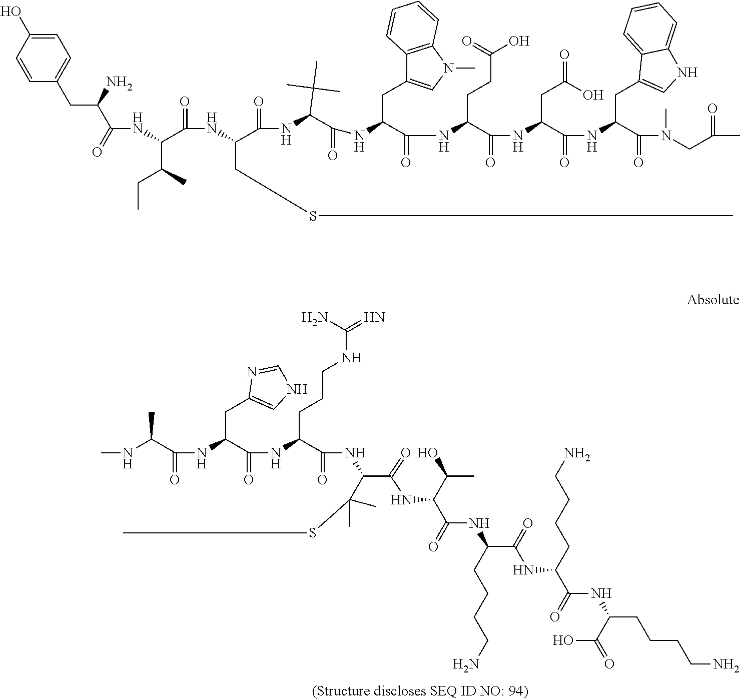

### Compound: 9

MW (calculated): 2191.9455 Da

Amino acid sequence:
(SEQ ID NO: 95)
DTyr-I-C(*)-Tle-TrpMe-bhAsn-D-W-Sar-A-H-R
-PEN(*)-DThr-DgGlu-DgGlu-DgGlu-OH

Synthesis and cyclization methods: S02, C02
Analytical method: A02
Rt: 17.24 min
Observed mass: 1096.975 Da, [M+2H]/2

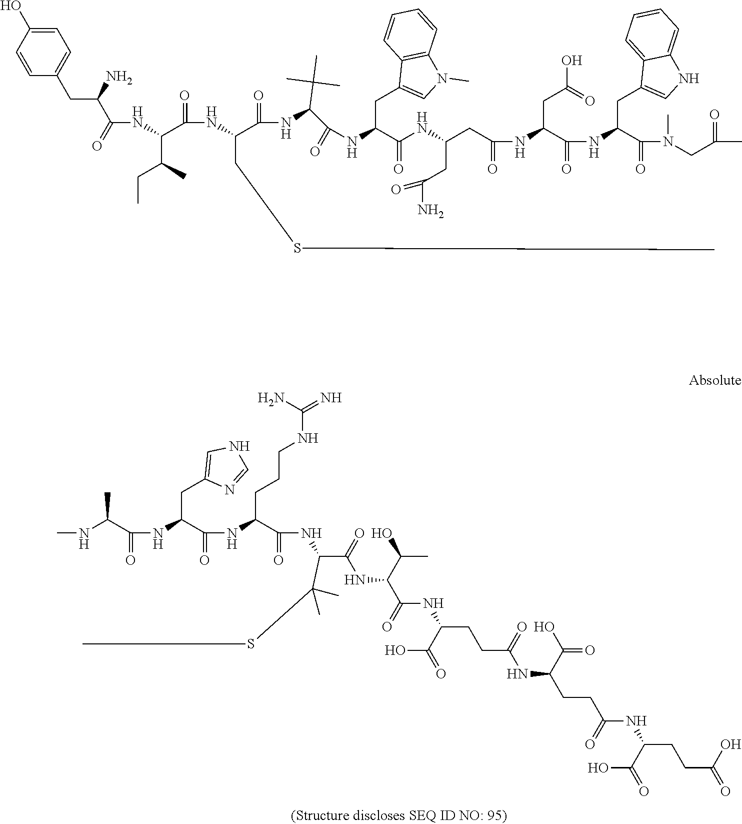

### Compound: 96

MW (calculated): 2216.1499 Da

Amino acid sequence:
(SEQ ID NO: 96)
DTyr-I-C(*)-Tle-TrpMe-bhAsn-D-W-Sar-A-H-R
-PEN(*)-DLys-DLys-DLys-DLys-OH

Synthesis and cyclization methods: S02, C02
Analytical method: A02
Rt: 14.83 min
Observed mass: 1109.078 Da, [M+2H]/2

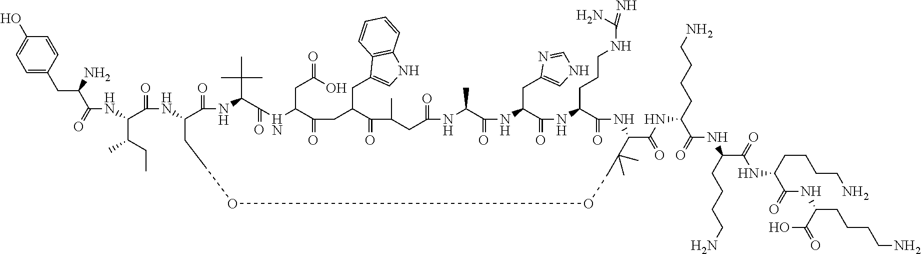

### Compound: 97

MW (calculated): 2247.1081 Da

Amino acid sequence:
(SEQ ID NO: 97)
DTyr-I-C(*)-Tle-TrpMe-DprAc-D-W-Sar-E-H-R
-PEN(*)-DThr-DLys-DLys-DLys-OH

Synthesis and cyclization methods: S02, C02
Analytical method: A02
Rt: 17.40 min
Observed mass: 1124.558 Da, [M+2H]/2

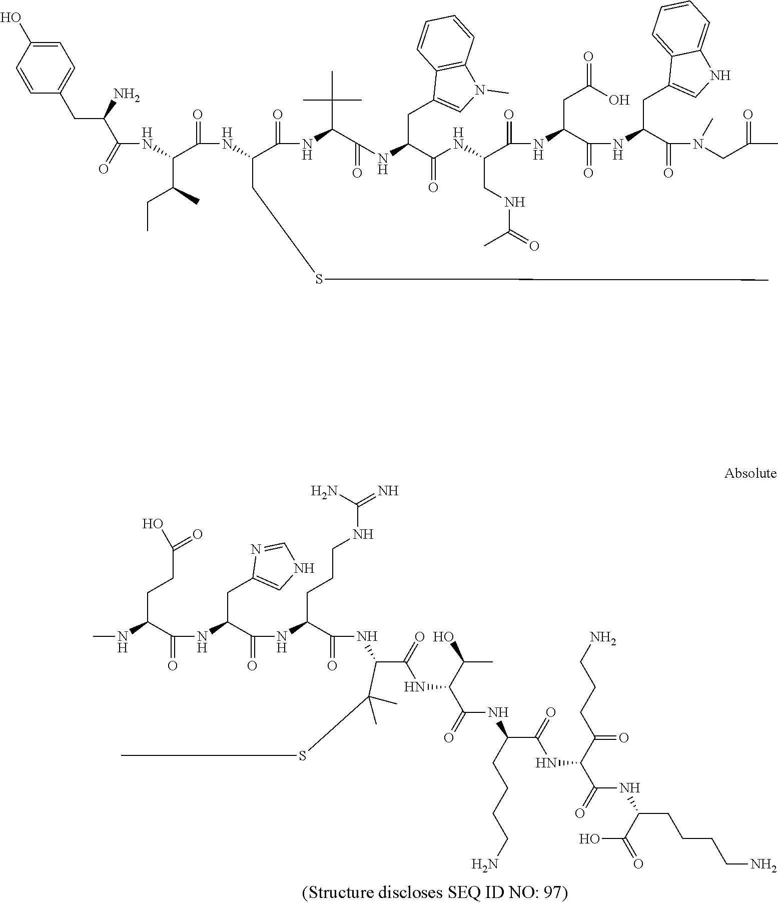

### Compound: 98

MW (calculated): 2062.9030 Da

Amino acid sequence:
(SEQ ID NO: 98)
DTyr-I-C(*)-Tle-TrpMe-bhAsn-D-W-Sar-A-H-R
-PEN(*)-DThr-gGlu-gGlu-OH

Synthesis and cyclization methods: S02, C02
Analytical method: A02
Rt: 22.52 min
Observed mass: 1032.450 Da, [M+2H]/2

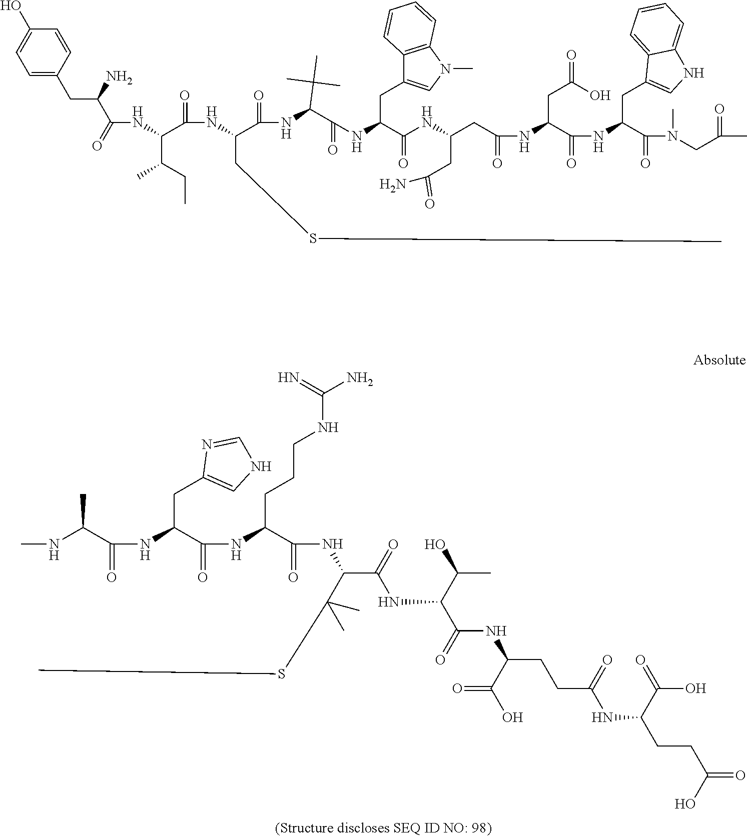

### Compound: 99

MW (calculated): 1933.8604 Da

Amino acid sequence:
(SEQ ID NO: 99)
DTyr-I-C(*)-Tle-TrpMe-bhAsn-D-W-Sar-A-H-R
-PEN(*)-DThr-gGlu-OH

Synthesis and cyclization methods: S02, C02
Analytical method: A02
Rt: 22.80 min
Observed mass: 967.930 Da, [M+2H]/2

### Compound: 100

MW (calculated): 2088.0550 Da

Amino acid sequence:
(SEQ ID NO: 100)
DTyr-I-C(*)-Tle-TrpMe-bhAsn-D-W-Sar-A-H-R
-PEN(*)-DLys-K-K-OH

Synthesis and cyclization methods: S02, C02
Analytical method: A02
Rt: 19.98 min
Observed mass: 1045.030 Da, [M+2H]/2

### Compound: 101

MW (calculated): 1959.9600 Da

Amino acid sequence:
(SEQ ID NO: 113)
DTyr-I-C(*)-Tle-TrpMe-bhAsn-D-W-Sar-A-H-R
-PEN(*)-DLys-K-OH

Synthesis and cyclization methods: S02, C02
Analytical method: A02
Rt: 20.70 min
Observed mass: 980.980 Da, [M+2H]/2
   Compound: 102 (no part of the invention)
MW (calculated): 1804.8178 Da

Amino acid sequence:
(SEQ ID NO: 114)
DTyr-I-C(*)-Tle-TrpMe-bhAsn-D-W-Sar-A-H-R
-PEN(*)-DThr-OH

Synthesis and cyclization methods: S02, C02
Analytical method: A02
Rt: 23.08 min
Observed mass: 903.410 Da, [M+2H]/2
   Compound: 103 (no part of the invention)
MW (calculated): 1831.8651 Da

Amino acid sequence:
(SEQ ID NO: 115)
DTyr-I-C(*)-Tle-TrpMe-bhAsn-D-W-Sar-A-H-R
-PEN(*)-DLys-OH

Synthesis and cyclization methods: S02, C02
Analytical method: A02
Rt: 21.49 min
Observed mass: 916.930 Da, [M+2H]/2

1. A compound of formula (Ia),
(Ia)
(SEQ ID NO: 117)
Y2-Z2-X0-X1-X2-X3-X4-X5-X6-X7-X8-X9-
X10-X11-X12-X13-Z1-Y1
or salt thereof, wherein
X0 is DTyr,
X1 is I,
X2 is C,
X3 is Tle,
X4 is TrpMe,
X5 is bhAsn or DprAc,
X6 is D,
X7 is W,
X8 is Sar,
X9 is A or E,
X10 is H,
X11 is R,
X12 is PEN,
X13 is selected from the group consisting of DArg, DLys, DThr, DPhe, P, and NMeF,
wherein the compound has a disulphide bridge between the sulphur atoms of X2 and X12,
and wherein
(a) Z1 is a sequence of n amino acid residues independently selected from a group consisting of K, E, and gGlu, DLys and DgGlu, n is an integer selected from 1 to 4, Y1 is OH, Z2 is absent, and Y2 is hydrogen; or
(b) Z1 is absent, Y1 is selected from the group consisting of OH, K—OH, and NH2, Z2 is a sequence of m amino acid residues independently selected from a group consisting of K, E, and gGlu, m is an integer selected from 1 to 4, and Y2 is hydrogen (SEQ ID NO: 118);
wherein
Tle refers to the residue of L-tert-butylglycine,
TrpMe refers to the residue of 1-methyl-L-tryptophan,
bhAsn refers to the residue of beta-homo-L-asparagine,
DprAc refers to the residue of (2S)-2-amino-3-acetamidopropanoic acid,
Sar refers to the residue of sarcosine,
PEN refers to the residue of L-penicillamine,
NMeI refers to the residue of N-methyl-L-isoleucine,
NMeF refers to the residue of N-methyl-L-phenylalanine,
APCA refers to the residue of 4-amino-piperidine-4 carboxylic acid, and
gGlu refers to the residue of gamma-L-glutamate.

2. The compound according to claim 1, wherein X5 is bhAsn; or a salt thereof.

3. The compound according to claim 1, wherein X5 is DprAc; or a salt thereof.

4. The compound according to claim 1, wherein
(a) Z1 is a sequence of n identical amino acid residues selected from a group consisting of K, E, gGlu, DLys, and DgGlu, n is an integer equal to two or three, Y1 is OH, Z2 is absent, and Y2 is hydrogen; or
(b) Z1 is absent, Y1 is selected from the group consisting of OH and K—OH, Z2 is a sequence of m identical amino acid residues selected from a group consisting of K, E, and gGlu, m is an integer equal to three, and Y2 is hydrogen;
or a salt thereof.

5. The compound according to claim 1, wherein Z1 is a sequence of n amino acid residues independently selected from the group consisting of K, E, gGlu, DLys and DgGlu, n is an integer equal to two or three, Y1 is OH, Z2 is absent, and Y2 is hydrogen; preferably n is an integer equal to three; or a salt thereof.

6. The compound according to claim 1, wherein X5 is bhAsn, Z1 is a sequence of n amino acid residues independently selected from the group consisting of K, E, gGlu, DLys and DgGlu, n is an integer equal to two or three, Y1 is OH, Z2 is absent, and Y2 is hydrogen; preferably n is an integer equal to three; or a salt thereof.

7. The compound according to claim 1, wherein X5 is DprAc, Z1 is a sequence of n amino acid residues independently selected from the group consisting of K, E, gGlu, DLys and DgGlu, n is an integer equal to two or three, Y1 is OH, Z2 is absent, and Y2 is hydrogen; preferably n is an integer equal to three; or a salt thereof.

8. The compound according to claim 5, wherein Z1 is a sequence of n identical amino acid residues, wherein the amino acid residue is selected from the group consisting of K, E, and gGlu, and n is an integer equal to two or three; preferably n is an integer equal to three; or a salt thereof.

9. The compound according to claim 5, wherein Z1 is a sequence of three identical amino acid residues wherein the amino acid residue is DLys or DgGlu; or a salt thereof.

10. The compound according to claim 1, wherein X13 is selected from the group consisting of DArg, DLys, and DThr; preferably DLys or DThr; or a salt thereof.

11. The compound according to claim 2, wherein X5 is bhAsn, and X9 is A; or a salt thereof.

12. The compound according to claim 3, wherein X5 is DprAc, and X9 is E; or a salt thereof.

13. The compound according to claim 1, wherein the compound is selected from the group consisting of Compounds according to the following table

| | | SEQ |
|---|---|---|
| Compound | | ID |
| No | Compound formula | NO |
| 3 | DTyr-I-C(*)-Tle-TrpMe-DprAc-D-W-Sar-A-H-R-PEN(*)-DArg-K-OH | 3 |
| 44 | DTyr-I-C(*)-Tle-TrpMe-bhAsn-D-W-Sar-A-H-R-PEN(*)-DThr-gGlu-gGlu-gGlu-OH | 44 |
| 50 | DTyr-I-C(*)-Tle-TrpMe-DprAc-D-W-Sar-A-H-R-PEN(*)-DArg-K-K-K-OH | 50 |
| 51 | DTyr-I-C(*)-Tle-TrpMe-DprAc-D-W-Sar-A-H-R-PEN(*)-DArg-gGlu-gGlu-gGlu-OH | 51 |
| 53 | DTyr-I-C(*)-Tle-TrpMe-DprAc-D-W-Sar-A-H-R-PEN(*)-DThr-gGlu-gGlu-gGlu-OH | 53 |
| 63 | DTyr-I-C(*)-Tle-TrpMe-DprAc-D-W-Sar-A-H-R-PEN(*)-DThr-K-K-K-OH | 63 |
| 64 | DTyr-I-C(*)-Tle-TrpMe-DprAc-D-W-Sar-A-H-R-PEN(*)-DLys-K-K-K-OH | 64 |
| 66 | DTyr-I-C(*)-Tle-TrpMe-DprAc-D-W-Sar-A-H-R-PEN(*)-NMeF-K-K-K-OH | 66 |
| 67 | DTyr-I-C(*)-Tle-TrpMe-DprAc-D-W-Sar-A-H-R-PEN(*)-DPhe-K-K-K-OH | 67 |
| 68 | DTyr-I-C(*)-Tle-TrpMe-DprAc-D-W-Sar-E-H-R-PEN(*)-DThr-K-K-K-OH | 68 |
| 69 | DTyr-I-C(*)-Tle-TrpMe-DprAc-D-W-Sar-E-H-R-PEN(*)-DLys-K-K-K-OH | 69 |
| 70 | DTyr-I-C(*)-Tle-TrpMe-DprAc-D-W-Sar-E-H-R-PEN(*)-P-K-K-K-OH | 70 |
| 71 | DTyr-I-C(*)-Tle-TrpMe-DprAc-D-W-Sar-E-H-R-PEN(*)-DPhe-K-K-K-OH | 71 |
| 72 | DTyr-I-C(*)-Tle-TrpMe-bhAsn-D-W-Sar-A-H-R-PEN(*)-DThr-K-K-K-OH | 72 |
| 73 | DTyr-I-C(*)-Tle-TrpMe-bhAsn-D-W-Sar-A-H-R-PEN(*)-DLys-K-K-K-OH | 73 |
| 74 | DTyr-I-C(*)-Tle-TrpMe-bhAsn-D-W-Sar-A-H-R-PEN(*)-P-K-K-K-OH | 74 |
| 75 | DTyr-I-C(*)-Tle-TrpMe-bhAsn-D-W-Sar-A-H-R-PEN(*)-DArg-K-K-K-OH | 75 |
| 76 | DTyr-I-C(*)-Tle-TrpMe-bhAsn-D-W-Sar-A-H-R-PEN(*)-DPhe-K-K-K-OH | 76 |
| 77 | DTyr-I-C(*)-Tle-TrpMe-bhAsn-D-W-Sar-E-H-R-PEN(*)-DThr-K-K-K-OH | 77 |
| 78 | DTyr-I-C(*)-Tle-TrpMe-bhAsn-D-W-Sar-E-H-R-PEN(*)-DLys-K-K-K-OH | 78 |
| 80 | DTyr-I-C(*)-Tle-TrpMe-bhAsn-D-W-Sar-E-H-R-PEN(*)-DPhe-K-K-K-OH | 80 |
| 81 | K-K-K-DTyr-I-C(*)-Tle-TrpMe-DprAc-D-W-Sar-A-H-R-PEN(*)-DArg-OH | 81 |
| 82 | K-K-K-DTyr-I-C(*)-Tle-TrpMe-DprAc-D-W-Sar-A-H-R-PEN(*)-DThr-OH | 82 |
| 83 | K-K-K-DTyr-I-C(*)-Tle-TrpMe-DprAc-D-W-Sar-A-H-R-PEN(*)-DLys-OH | 83 |
| 84 | K-K-K-DTyr-I-C(*)-Tle-TrpMe-DprAc-D-W-Sar-A-H-R-PEN(*)-P-OH | 84 |
| 85 | K-K-K-DTyr-I-C(*)-Tle-TrpMe-DprAc-D-W-Sar-A-H-R-PEN(*)-DPhe-OH | 85 |
| 86 | K-K-K-DTyr-I-C(*)-Tle-TrpMe-DprAc-D-W-Sar-E-H-R-PEN(*)-DArg-OH | 86 |
| 87 | K-K-K-DTyr-I-C(*)-Tle-TrpMe-DprAc-D-W-Sar-E-H-R-PEN(*)-DThr-OH | 87 |
| 88 | K-K-K-DTyr-I-C(*)-Tle-TrpMe-DprAc-D-W-Sar-E-H-R-PEN(*)-DLys-OH | 88 |
| 89 | K-K-K-DTyr-I-C(*)-Tle-TrpMe-bhAsn-D-W-Sar-E-H-R-PEN(*)-DArg-OH | 89 |
| 90 | K-K-K-DTyr-I-C(*)-Tle-TrpMe-bhAsn-D-W-Sar-E-H-R-PEN(*)-DThr-OH | 90 |
| 91 | K-K-K-DTyr-I-C(*)-Tle-TrpMe-bhAsn-D-W-Sar-E-H-R-PEN(*)-DLys-OH | 91 |
| 92 | K-K-K-DTyr-I-C(*)-Tle-TrpMe-bhAsn-D-W-Sar-E-H-R-PEN(*)-P-OH | 92 |
| 93 | K-K-K-DTyr-I-C(*)-Tle-TrpMe-bhAsn-D-W-Sar-E-H-R-PEN(*)-DPhe-OH | 93 |
| 95 | DTyr-I-C(*)-Tle-TrpMe-bhAsn-D-W-Sar-A-H-R-PEN(*)-DThr-DgGlu-DgGlu-DgGlu-OH | 95 |
| 96 | DTyr-I-C(*)-Tle-TrpMe-bhAsn-D-W-Sar-A-H-R-PEN(*)-DLys-DLys-DLys-DLys-OH | 96 |
| 97 | DTyr-I-C(*)-Tle-TrpMe-DprAc-D-W-Sar-E-H-R-PEN(*)-DThr-DLys-DLys-DLys-OH | 97 |
| 98 | DTyr-I-C(*)-Tle-TrpMe-bhAsn-D-W-Sar-A-H-R-PEN(*)-DThr-gGlu-gGlu-OH | 98 |
| 99 | DTyr-I-C(*)-Tle-TrpMe-bhAsn-D-W-Sar-A-H-R-PEN(*)-DThr-gGlu-OH | 99 |
| 100 | DTyr-I-C(*)-Tle-TrpMe-bhAsn-D-W-Sar-A-H-R-PEN(*)-DLys-K-K-OH | 100 |
| 101 | DTyr-I-C(*)-Tle-TrpMe-bhAsn-D-W-Sar-A-H-R-PEN(*)-DLys-K-OH | 113 |

wherein C(*) and PEN(*) indicate the pairs of amino acid residues X2 and X12 bridged between the sulphur atoms of their side chains, forming a disulphide bond.

14. The compound according to claim 1, wherein the compound is in the form of a pharmaceutically acceptable salt.

15. A pharmaceutical composition comprising the compound of claim 1 together with one or more pharmaceutically acceptable carrier and/or excipients.

16. A method of treating or preventing an eye or ocular disease in a patient, wherein the method comprises administering to the patient in need thereof a therapeutically effective amount of a compound according to claim 1, wherein the eye or ocular disease is selected from the group consisting of retinopathy, proliferative retinopathy (PR), retinopathy of prematurity, ischemic retinopathy, diabetic retinopathy (DR), proliferative diabetic retinopathy (PDR), non-proliferative diabetic retinopathy, diabetic macular edema (DME), diabetic macular ischemia (DMI), age-related macular degeneration (AMD), dry age-related macular degeneration (AMD), wet age-related macular degeneration (AMD), geographic atrophy (GA), retinitis pigmentosa, inherited retinal dystrophy, myopic degeneration, retinal vein occlusions, retinal artery occlusions, endophthalmitis, uveitis, cystoid macular edema, choroidal neovascular membrane secondary to any retinal diseases, optic neuropathies, glaucoma, neovascular glaucoma (NVG), retinal detachment, toxic retinopathy, radiation retinopathy, traumatic retinopathy, drug-induced retinal vasculopathy, retinal neovascularisation, polypoidal choroidal vasculopathy, retinal vasculitis, retinal microaneurysm, retrolental fibroplasia, chorioretinitis, Fuch's dystrophy, macular telangiectasia, Usher syndrome, Stargardt disease, and neuromyelitis optica spectrum disorder.

17. A method of treating or prevention of a disease in a patient, wherein the method comprises administering to the patient in need thereof a therapeutically effective amount of a compound according to claim 1, wherein the disease is selected from the group consisting of:
periodontitis, rheumatoid arthritis, spinal cord injury, stroke, multiple sclerosis;
Parkinson's disease, Alzheimer's disease; cancer;
respiratory disorders, asthma, chronic obstructive pulmonary disease (COPD), allergic inflammation, emphysema, respiratory distress syndrome (RDS) neonatal and adult, rhinitis, sinusitis;
bacterial infections, sepsis, ischemia-reperfusion injury in various tissues, myocardial infarction, anaphylaxis, paroxysmal nocturnal hemoglobinuria (PNH), autoimmune hemolytic anemias, psoriasis, hidradentitis suppurativa, myasthenia gravis, systemic lupus erythematosus, CHAPLE syndrome, C3 glomeropathy, IgA nephropathy, atypical hemolytic uremic syndrome, Crohn's disease, ulcerative colitis and antiphospholipid syndrome, endometriosis, systemic sclerosis, and primary immune-complex membranoproliferative glomerulonephritis.
